SEQUENCE LISTING (1 sequence)

SEQ ID NO: 1 - protein, 5 aa, Homo sapiens
LSGGQ

(19) United States
(12) Patent Application Publication
(10) Pub. No.: US 2004/0214848 A1
(43) Pub. Date: Oct. 28, 2004

(54) Inhibitors of ABC drug transporters in microbial cells

(76) Inventor: Grant L. Schoenhard, San Carlos, CA (US)

Correspondence Address: Janet M. McNicholas, Ph.D., McAndrews, Held & Malloy, Ltd., 34th Floor, 500 West Madison Street, Chicago, IL, 60661, US

(21) Appl. No.: 10/159,212

(22) Filed: May 30, 2002

Related U.S. Application Data
(63) Continuation-in-part of application No. 10/000,107, filed on Oct. 30, 2001.

(51) Int. Cl. A61K 31/485

(52) U.S. Cl. 514/282

(57) ABSTRACT

The present invention relates to microbial infections, including those involving multidrug resistance and, in particular, to opioid compounds that are inhibitors of drug transporters of the ABC protein superfamily. The invention relates to methods of treating microbial infections using anti-microbial agents and opioid inhibitors of such transporters. The invention also relates to methods for selecting or identifying compounds for the ability to inhibit drug transporter proteins and to methods of inhibiting drug transporter proteins. The invention concerns the new use of opioid receptor antagonists in the treatment of microbial infections, including multidrug resistant microbial infections.

### CROSS-REFERENCE TO RELATED APPLICATIONS

This application is a continuation-in-part of co-pending U.S. application Ser. No. 10/000,107, filed Oct. 30, 2001, hereby incorporated by reference.

### BACKGROUND

Drug resistance plays a crucial role in the failure of drug therapy for various infections and infectious diseases. Resistance may be mediated by efflux mechanisms that pump anti-microbial agents, such as anti-bacterials or antifungals, out of the microbial cell before these agents elicit their effects. These resistance systems are characteristically energy-dependent and may be either primary or secondary active transport systems. Such systems include microbial ATP-binding cassette transporter systems.

ATP-binding cassette (ABC) proteins play a central role in living cells through their role in nutrient uptake, protein, drug and antibiotic secretion, osmoregulation, antigen presentation, signal transduction and others. The majority of ABC proteins have a translocation function either in import of substrates or secretion of cellular products or xenobiotics.

The ATP binding (ABC) superfamily is one of the largest superfamilies known. With the multiplication of genome sequencing projects, new sequences appear every week in the GenBank database. Members of this family possess a highly conserved protein or module, the ABC module, that displays the WalkerA and WalkerB motifs separated by a short highly conserved sequence (consensus LSGGQ, SEQ ID NO: 1) called a signature sequence or linker peptide. Most ABC cassette proteins are primary transporters for movement of molecules across biological membranes. The substrates handled by these transporters are extraordinarily varied ranging from small molecules to macromolecules.

ABC proteins of particular interest are the drug transporters associated with multidrug resistance in microbial cells. The family of drug transporters includes two different subfamilies, the multidrug resistance (MDR) proteins, such as PGP, and the multidrug resistance-associated protein (MRP) family. The human multidrug resistance-associated protein family is composed of a number of well-characterized members. (See, e.g., Borst et al, J. Natl Cancer Inst. 92:1295-1302 (2000)). Originally implicated in the resistance of tumor cells to chemotherapeutic agents, the multi-drug resistance protein P-glycoprotein (PGP), a product of the MDR1 gene, belongs to the ATP-binding cassette family of proteins. P-glycoprotein is an ATP-dependent drug transporter that is predominantly found in the apical membranes of a number of epithelial cell types in the body, including the luminal membrane of the brain capillary endothelial cells that make up the blood-brain barrier. PGP is expressed in the human intestine, blood brain barrier, liver, and other tissues. (See, e.g., Matheny et al., Pharmacology 21(7): 778-796 (2001); Nolnar et al., Anticancer Res. 17(1A): 481-486 (1997)); van Veen et al., Nature 391 (6664): 291-295 (1998); Higgins, Res. Microbiol. 152 (3-4): 205-210 (2001); van Veen et al., Pharmacol. Ther. 85(3): 245-249 (2000). Expression of PGP, localized to cell membranes may affect the bioavailability of drug molecules that are substrates for this transporter. Drugs that inhibit P-glycoprotein can alter the absorption, disposition and elimination of co-administered drugs and can enhance bioavailability or cause unwanted drug-drug interactions. Interaction with PGP can be studied using either direct assays of drug transport in polarized cell systems or with indirect assays such as drug-stimulated ATPase activity and inhibition of the transport of fluorescent substrates.

Multidrug resistance, mediated by ABC proteins, is also very common among microbial organisms, particularly in those that infect humans and agricultural products. 30% or more hospital patients are treated with one or more courses of anti-microbial therapy. The inevitable consequence of the widespread use of anti-microbial therapy has been the emergence of antibiotic resistant, and even more problematically multidrug resistant, microbial pathogens. Microbes (i.e., microbial organisms or microbial cells) can develop resistance to anti-microbial agents by several different methods, including inactivating the anti-microbial agent or stopping the anti-microbial agent from reaching its target. In the case of multidrug resistance, a common method of developing resistance is by stopping the agent from reaching its site of action. A particularly common method is by actively exporting the anti-microbial agent from the cell after it has entered by any number of methods including passive diffusion across the membrane or protein-mediated transport across the cell membrane. Host transporters mediate transport of numerous compounds in the duodenum, the liver, the kidneys, the brain, and putatively in other tissues. These host transporters have the capacity to transport drugs, drug conjugates and drug complexes across plasma membranes into extracellular fluids or back into associated tissues.

Cystic fibrosis (CF) is the most common lethal inherited disorder among Caucasian populations, affecting between 1 in 2000 to 1 in 4500 children. CF is a recessive disorder resulting from a defect in the cystic fibrosis transmembrane conductance regulator (CFTR) gene, a member of the ATP binding cassette (ABC) superfamily, located on a long arm of chromosome seven, that is thought to encode a cAMP-regulated chloride ion channel. CF is characterized by chronic pulmonary infection and colonization of the lungs by gram-negative bacteria (predominately Pseudomonas aeruginosa), pulmonary inflammation, and progressive pulmonary damage, as well as pancreatic insufficiency. There is prominent pulmonary neutrophil infiltration, and levels of the neutrophil enzyme elastase found in the sputum of CF patients are so high as to overwhelm the host's elastase inhibitor-antitrypsin. In addition, CF is associated with various extra-pulmonary autoimmune phenomena, including arthropathy, liver disease resembling sclerosing cholangitis, and both cutaneous and systemic vasculitis. Due to improvements in therapy, more than 25% of the patients reach adulthood and more than 9% live past the page of 30. Harrison's Principles of Internal Medicine, 13^{th} ed., Isselbacher et al., eds., McGraw-Hill, NY. Many strains of P. aeruginosa have developed resistance to a broad range of antibiotics and thus have been epidemic within the population of CF patients. This phenomenon is particularly problematic in chronic care facilities where CF patients are clustered.

Multidrug resistance is also a problem in treating protozoan parasite infestations of humans. For example, malaria, the worlds most deadly parasitic disease, has become particularly difficult to treat due to parasite resistance to antimalarial drugs such as chloroquine. Chloroquine-resistant and -sensitive Plasmodium falciparum strains accumulate chloroquine at equivalent rates, but the chloroquine-resistant strains efflux the drug 40-50 times more rapidly than chloroquine-sensitive strains. The malarial P-glycoprotein homologue, Pghl, has been shown to be involved in resistance to chloroquine, mefloquine and halfantrine. Drug efflux can be inhibited by the classic PGP inhibitors, verapamil and diltiazem. Leshmaniasis is the second leading cause of death caused by protozoan parasites, mainly due to resistance to conventional drugs. In Leishmania, P-glycoprotein-like transporters have been involved in a multidrug resistance phenotype, including resistance to daunomycin, vinblastine and adriamycin.

Fungal infections are becoming a major health concern for a number of reasons, including the limited number of antifungal agents available, the increasing incidence of species resistant to older antifungal agents, and the growing population of immunocompromised patients at risk for opportunistic fungal infections. The incidence of systemic fungal infections increased 600% in teaching hospitals and 220% in non-teaching hospitals during the 1980's. The most common clinical isolate is Candida albicans, a potent fungal pathogen in immunocompromised hosts (comprising about 19% of all isolates). The incidence of Candida albicans acquiring resistance to antifungals like azoles has increased considerably in recent years. Overexpression of CDR1, an ABC family member, has been implicated in the development of antifungal resistance in C. albicans. In one study, nearly 40% of all deaths from hospital-acquired infections were due to fungi. Sternberg, Science, 266:1632-1634 (1994). The ability of the drug transporter proteins such as ABC proteins to actively transport therapeutic substances from cells, including both microbial and host organs, tissues or cells, has impeded the development of therapies for a wide variety of disorders and conditions in multicellular hosts, particularly in humans. Thus, a continuing need exists for methods to increase the ability of clinicians to administer bioactive substances across biological membranes.

### SUMMARY OF THE INVENTION

The present invention provides methods and compositions with anti-microbial agents and opioid inhibitors of ABC drug transporters. Exemplary opioid inhibitors useful in such methods and compositions are nalmefene, naltrexone and naloxone. Additionally, opioid inhibitors of the invention are described having a pharmacophore as defined herein. Opioid inhibitors of the invention are also described as having a structure of a formula as defined herein.

The present invention relates to methods of treating microbial infections, including those involving multidrug resistance, and to the use of opioid compounds, including opioid receptor antagonists, as inhibitors of drug transporters of the ABC protein superfamily. The present invention provides methods of increasing the potency of an anti-microbial agent by co-administering to a subject (e.g., host) infected with a microbe (e.g., microbial organism or microbial cell), a dose, including a therapeutic or a sub-therapeutic dose, of an anti-microbial agent and a dose of an opioid inhibitor of the ABC drug transporter. For example, the anti-microbial agent is a substrate of an ABC drug transporter and the dose of the opioid inhibitor of the ABC drug transporter is sufficient to reduce efflux of the anti-microbial agent from the microbe, increase the intracellular concentration of the anti-microbial agent in the microbe, and/or inhibit a host drug transporter. Also, for example, the dose of an opioid inhibitor of the ABC transporter facilitates the distribution of anti-microbial agents into tissues and/or cells of a subject where, in the absence of the inhibitor, the uninhibited ABC transporter facilitated efflux is so high as to prevent attainment of therapeutic concentrations of anti-microbial agents n those tissues and/or cells.

The invention provides methods for decreasing toxicity and/or side effect associated with administration of an anti-microbial agent to a subject by co-administering a dose, including a therapeutic or sub-therapeutic dose, of an anti-microbial agent and a dose of an opioid inhibitor of an ABC drug transporter. For example, the dose of opioid inhibitor is sufficient to reduce efflux of the anti-microbial agent from the microbe, increase the intracellular concentration of the anti-microbial agent in the microbe and/or inhibit a host drug transporters. Also, for example, the dose of an opioid inhibitor of the ABC transporter facilitates the distribution of anti-microbial agents into tissues and cells of a subject where, in the absence of the inhibitor, the uninhibited ABC transporter facilitated efflux is so high as to prevent attainment of therapeutic concentrations of anti-microbial agents in those tissues and/or cells.

The invention also provides compositions for treating microbial infection with a combination of an anti-microbial agent and an opioid inhibitor of a ABC drug transporter. For example, the anti-microbial agent is a substrate of the ABC drug transporter. The invention provides an opioid inhibitor of the ABC drug transporters that have a pharmacophore defined by a hydrogen bonding moiety at a three-dimensional location corresponding to the hydroxyl at position 3 of naltrexone, a hydrogen bonding moiety at a three-dimensional location corresponding to the hydroxyl at position 14 of naltrexone, a hydrophobic moiety at a three-dimensional location corresponding to the cyclopropyl moiety appended to the nitrogen of naltrexone, and a region of electron density at a three-dimensional location corresponding to the ethylene moiety at 6-position of naltrexone. Additionally, the invention provides ABC drug transporter inhibitors of the formula:

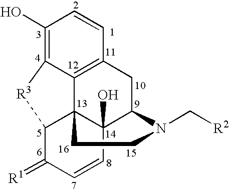

wherein R¹ is CH₂ or O;

wherein R² is a cycloalkyl, unsubstituted aromatic, alkyl or alkenyl; and

wherein R³is O, CH₂ or NH.

Exemplary opioid inhibitors of ABC transporters are nalmefene, naltrexone and naloxone.

The invention also provides includes methods of enhancing the anti-microbial activity of an anti-microbial agent against a microbe by contacting the microbe with the anti-microbial agent and an opioid inhibitor of an ABC drug transporter in an amount effective to inhibit a drug transporter in the microbe. For example, the microbe expresses an ABC drug transporter and the anti-microbial agent is a substrate of the ABC drug transporter.

The invention also provides methods of suppressing growth of a microbe expressing an ABC drug transporter protein comprising contacting the microbe with a sub-therapeutic amount of an anti-microbial agent in the presence of an opioid inhibitor of the ABC drug transporter.

The invention also provide methods of inhibiting a microbial P-glycoprotein homologue in a subject suffering from a microbial infection. A P-glycoprotein inhibiting amount of an inhibitor of an ABC transporter, preferably of nalmefene, naltrexone or naloxone, is administered to the subject before, with, or after the administration to the subject of a therapeutic or sub-therapeutic amount of an anti-microbial agent.

The invention also provides compositions for the treatment of a microbial infection comprising an opioid inhibitor of an ABC drug transporter and an anti-microbial agent.

The invention also provides methods of identifying compounds, including anti-microbial compounds, for improved treatment of microbial infections. The method includes identifying an anti-microbial agent, assaying the ability of the therapeutic agent to be transported across a membrane by an ABC protein, and repeating the transport assay to determine whether addition of an opioid inhibitor of an ABC drug transporter inhibits transport of the therapeutic agent across the membrane. The desired compound is identified as a compound that is transported by an ABC protein and whose ABC protein-mediated transport is inhibited by an opioid inhibitor. The desired compound inhibits the ABC transporter in various tissues and/or cells of a subject that prevents the attainment of therapeutic concentrations of anti-microbial agents in those tissues and/or cells. The invention provides such anti-microbial compounds.

The invention also provides methods of enhancing the potency of an anti-microbial agent by co-administering an amount, including a therapeutic or a sub-therapeutic amount of the anti-microbial agent and an amount of an opioid inhibitor of an ABC transporter, including an amount sufficient to reduce transport of the anti-microbial agent across a biological membrane.

The invention provides methods for screening for an opioid inhibitor of an ABC drug transporter by determining whether a potential opioid inhibitor inhibits growth of a microbial cell in the presence of an amount, including a therapeutic or a sub-therapeutic amount, of anti-microbial agent. Inhibition of growth is assayed by comparing the growth of a microbial cell which expresses the ABC drug transporter, with growth of a second microbial cell which does not produce the ABC drug transporter. Both are grown in the presence of an amount, including a therapeutic or a sub-therapeutic amount, of the anti-microbial agent.

The invention also provides methods for screening for an opioid inhibitor of an ABC drug transporter. The method includes contacting a potential opioid inhibitor of an ABC drug transporter protein with the ABC drug transporter protein in the presence of a compound that is a known opioid inhibitor, including, for example, nalmefene, naltrexone and naloxone, wherein the compound is detectable by any means of detection and measuring the amount of detected compound bound to the ABC drug transporter. The measured amount is compared to the amount of detectably labeled compound bound by the ABC drug transporter when the drug transporter is contacted with the compound alone. An ABC drug transporter inhibitor is identified by a decreased amount of labeled compound bound to the ABC drug transporter when the potential inhibitor is present. The compound may be detectably labeled (e.g., radiolabelled) or detected by spectroscopy (e.g., U.V., mass spectral, infrared, flame ionization, electrochemical) or other detectors capable of quantifying compounds alone or in tandem with chromatography.

The invention also provides methods of treating a microbial infection in a subject (e.g., an animal host, including a human) by administering an anti-microbial agent and an amount of an opioid inhibitor of an ABC drug transporter, including, for example, nalmefene, naltrexone or naloxone sufficient to increase the intracellular concentration of the anti-microbial agent. The ABC drug transporter inhibitor increases the susceptibility of the microbe to the anti-microbial agent. An amount of such opioid inhibitors also or alternatively inhibits the ABC transporter in various tissues and cells of the subject that prevents the attainment of therapeutic concentrations of anti-microbial agents in those tissues and cells.

### BRIEF DESCRIPTION OF THE DRAWINGS

FIG. 1 illustrates the chemical structures of nalmefene, naltrexone, naloxone, 6-β-naltrexol and nalorphine.

FIG. 2 presents an overlay of the opioid analogues, nalmefene, naltrexone, naloxone, 6-β-naltrexol and nalorphine.

FIG. 3A shows the molecular orbitals and electrostatic potential of nalmefene as calculated using Spartan (Wavefunction, Inc.).

FIG. 3B shows the molecular orbitals and electrostatic potential of naloxone as calculated using Spartan (Wavefunction, Inc.).

FIG. 4A-4H provide information about the 200 nearest neighbors to the opioid analogues examined in the QSAR analysis.

### DETAILED DESCRIPTION

The present invention is based in part on surprising results from transport studies that compounds previously identified as opioid receptor antagonists are inhibitors of ABC drug transporter proteins, such as the exemplary P-glycoprotein, PGP-1a. Opioid receptor antagonists, including, for example, naltrexone, have been clinically used for decades but their transport characteristics have never been evaluated using contemporary cultured cell lines technology. Administration of opioid receptor antagonists, such as naloxone, nalmefene and naltrexone, unexpectedly result in increased intracellular concentrations of co-administered therapeutic agents in cells expressing an ABC drug transporter protein, particularly in microbial cells expressing a homologue of PGP1a. The present invention provides a novel class of drug transporter inhibitors that act by inhibiting ABC transporter proteins and/or their associated ATPase as described herein. The ABC drug transporter inhibitors of the present invention are useful in the inhibition of microbial drug transportors as well as those transporters associated with the microbe's host. The invention further provides a pharmacophore that identifies new drug targets as inhibitors of ABC transporter proteins.

As used herein, the terms “transporter” and “drug transporter” refer to a protein for the carrier-mediated influx and efflux of drugs and endocytosis of biologically active molecules across a cell membrane barrier, including across a gut, liver, or blood-brain barrier. An inhibitor of a transporter is expected to increase the efficacy of an active agent according to the invention, wherein the transporter inhibitor reduces efflux across the cellular membrane of a microbial cell, increases influx into the microbial cell, and/or inhibits a host drug transporter. Preferably the drug transporter protein is a member of the ABC superfamily, referred to as an “ABC drug transporter.” The ABC drug transporter may either be a multidrug resistance protein (MDR) or a multidrug resistance-associated protein (MRP).

Most preferably the microbial ABC drug transporter is a homologue of human PGP1a. As used herein, the terms “PGP homologue” or “homologue of PGP1a” refers to an ABC transporter that shares at least 80% amino acid sequence identity to an ABC module of human P-glycoprotein 1a. More preferably the PGP homologue shares at least 90% amino acid sequence identity with an ABC module of a human P-glycoprotein 1a. Most preferably the PGP homologue shares at least 95% amino acid sequence identity with an ABC module human P-glycoprotein 1a.

Among the ABC superfamily of drug transporters, there are several closely conserved regions, the nucleotide binding motifs of the WalkerA region and WalkerB region, and the short consensus sequence (leucine-serine-glycine-glycine-glutamine, or LSGGQ, SEQ ID NO: 1). ABC drug transporters generally contain the consensus sequence or a very closely related sequence. The QSAR analysis of the present invention provides the very surprising result that the opioid receptor antagonists that act as ABC drug transporter inhibitors are expected to bind in the region of this LSGGQ consensus sequence, as well as additional binding regions. Thus, the present invention defines a strictly conserved inhibition site shared among all ABC drug transporter proteins. Therefore, the ABC drug transporter inhibitor, including compounds identified as opioid receptor antagonists, according to the present invention will function as an inhibitor of a ABC drug transporter protein that shares the LSGGQ conserved sequence.

Thus, the present invention is based up the identification of a new class of drug transporter inhibitors. The term “drug transporter inhibitor” or “ABC drug transporter inhibitor” refers to a compound that binds to an ABC drug transporter protein and inhibits, e.g., either completely blocks or merely slows transport of compounds across biological barriers. Compounds, such as drugs, that inhibit drug transporters can alter the absorption, disposition and elimination of co-administered drugs and can enhance bioavailability or cause unwanted drug-drug interactions. Interaction with drug transporters can be studied using either direct assays of drug transport in polarized cell systems and/or with indirect assays such as drug-stimulated ATPase activity or inhibition of the transport of fluorescent substrates. Drugs affected by the drug transporter, P-glycoprotein, include ondasetron, dexamethasone, domperidone, loperamide, doxorubicin, neifinavir, indinevir, sugguinavir, erythromycin, digoxin, vinblastine, paclitaxel, invermectin and cyclosporin. Known inhibitors of P-glycoprotein include ketoconazole, verapamil, quinidine, cyclosporin, digoxin, erythromycin and loperamide. The present invention unexpectedly identifies opioid receptor antagonists, such as nalmefene, naltrexone and naloxone, as potent inhibitors of the drug transporter, P-glycoprotein. The QSAR analysis of the invention demonstrates that the opioid receptor antagonists are also inhibitors of ABC drug transporters, especially of microbial homologues of human PGP1a. The present invention provides opioid inhibitors of the ABC transporters that have a pharmacophore defined by a hydrogen bonding moiety at a three-dimensional location corresponding to the hydroxyl at position 3 of naltrexone, a hydrogen bonding moiety at a three-dimensional location corresponding to the hydroxyl at position 14 of naltrexone, a hydrophobic moiety at a three-dimensional location corresponding to the cyclopropyl moiety appended to the nitrogen of naltrexone, and a region of electron density at a three-dimensional location corresponding to the ethylene moiety at 6-position of naltrexone.

An “opioid receptor antagonist” is an opioid compound or composition including any active metabolite of such compound or composition, that in a sufficient amount attenuates (e.g., blocks, inhibits, prevents or competes with) the action of an opioid receptor agonist. An opioid receptor antagonist binds to and blocks (e.g., inhibits) opioid receptors on nociceptive neurons. Opioid receptor antagonists include: nalmefene, naltrexone (e.g., marketed in 50 mg dosage forms as ReVia® or Trexan®), naloxone (e.g., marketed as Narcan®), methylnaltrexone, methiodide, nalorphine, naloxonazine, nalide, nalmexone, nalbuphine, nalorphine dinicotinate, naltrindole (NTI), naltrindole isothiocyanate (NTII), naltriben (NTB), nor-binaltorphimine (nor-BNI), β-funaltrexamine (β-FNA), BNTX, cyprodime, ICI-174,864, LY117413, MR2266, or an opioid receptor antagonist having the same pentacyclic nucleus as nalmefene, naltrexone, nalorphine, nalbuphine, thebaine, levallorphan, oxymorphone, butorphanol, buprenorphine, levorphanol meptazinol, pentazocine, dezocine, or their pharmacologically effective esters or salts. In some preferred embodiments, the opioid receptor antagonist is nalmefene, naltrexone, naloxone, or mixtures thereof.

The term “opioid” refers to compounds which bind to specific opioid receptors and have agonist (e.g., activation) or antagonist (e.g., inactivation) effects at these receptors, and thus are “opioid receptor agonists” or “opioid receptor antagonists,” respectively.

In particular, the present invention contemplates enhancing the efficacy of anti-microbial agents by co-administering the anti-microbial agent with an ABC transporter inhibitor such as an opioid receptor antagonist. The opioid receptor antagonists, nalmefene, naltrexone and naloxone, are particularly suited for the compositions and methods of the present invention. Although some inhibitors of ABC drug transportors are known in the art, many of these are toxic, especially if used repeatedly over a period of time. For example, when used orally, ketoconazole has been associated with hepatic toxicity, including some fatalities. The opioid receptor antagonists, however, have limited side effects, and particularly at the low concentrations administered in the present invention. Each of the opioid receptor antagonists nalmefene, naltrexone and naloxone have been administered for human use in antagonistically effective amounts for treatment of opioid overdose and addictions.

Co-administration of an opioid ABC drug transporter inhibitor and an anti-microbial agent is expected to provide more effective treatment of microbial infections. Concurrent administration of the two agents may provide greater therapeutic effects in vivo than the anti-microbial agent provides when administered singly. For example, concurrent administration may permit a reduction in the dosage of the microbial agent with achievement of a similar therapeutic effect. Alternatively, the concurrent administration may produce a more rapid or complete anti-microbial effect than could be achieved with the anti-microbial agent alone.

“Co-administer,” “co-administration,” “concurrent administration” or “co-treatment” refers to administration of an anti-microbial agent and an opioid drug transporter inhibitor, in conjunction or combination, together, or before or after each other. The anti-microbial agent and the opioid drug transporter inhibitor may be administered by different routes. For example, the antibiotic agent may be administered orally and the opioid drug transporter inhibitor intravenously, or vice versa. The antibiotic agent and the opioid drug transporter inhibitor are preferably both administered orally, as immediate or sustained release formulations. The antibiotic agent and opioid drug transporter inhibitor may be administered simultaneously or sequentially, as long as they are given in a manner to allow both agents to achieve effective concentrations to yield their desired therapeutic effects.

“Therapeutic effect” or “therapeutically effective” refers to an effect or effectiveness that is desirable and that is an intended effect associated with the administration of an active agent according to the invention. A “therapeutic amount” is the amount of an active agent sufficient to provide a therapeutic effect. “Sub-therapeutic amount” is an amount of the active agent which does not cause a therapeutic effect in a subject administered the active agent alone, but when used in combination with a opioid drug transporter inhibitor is therapeutically effective.

Therapeutic effectiveness is based on a successful clinical outcome, and does not require that the anti-microbial agent or agents kill 100% of the organisms involved in the infection. Success depends on achieving a level of anti-microbial activity at the site of infection that is sufficient to inhibit the microbe in a manner that tips the balance in favor of the host. When host defenses are maximally effective, the anti-microbial effect required may be minimal. Reducing organism load by even one log (a factor of 10) may permit the host's own defenses to control the infection. In addition, augmenting an early effect to kill and/or inhibit the growth of the microbe can be more important than a similar long-term effect. These early events are a significant and critical part of therapeutic success, because they allow time for host defense mechanisms to activate. Increasing the microbicidal rate may be particularly important for infections such as meningitis, bone or joint infections. Stratton, Antibiotics in Laboratory Medicine, 3rd ed. (Loftan, V., Ed.) pp. 849-879, Williams and Wilkins, Baltimore Md. (1991).

The ability of the inhibitor of an ABC drug transporter to improve the therapeutic effectiveness of anti-microbial agents, such as antibiotics, in vivo may be demonstrated in in vivo animal models, or may be predicted on the basis of a variety of in vitro tests, including (1) determinations of the minimum inhibitory concentration (MIC) of an anti-microbial required to inhibit growth of a microbe for 24 hours, (2) determinations of the effect of an anti-microbial on the kinetic growth curve of a microbe, and (3) checkerboard assays of the MIC of serial dilutions of anti-microbial in combination with serial dilutions of the inhibitor of the ABC drug transporter. Exemplary models or tests are described in Eliopoulos and Moellering In Antibiotics in Laboratory Medicine, 3rd ed. (Loftan, V., Ed.) pp. 432-492, Williams and Wilkins, Baltimore Md. (1991).

Using in vitro determinations of anti-microbial agent, such as an antibiotic, MIC at 24 hours, an inhibitor of an ABC drug transporter may be shown to reduce the MIC of the anti-microbial agent. With this result, it is expected that concurrent administration of the inhibitor of an ABC drug transporter in vivo will increase susceptibility of the microbe to the anti-microbial agent. An ABC transporter inhibitor may also be shown to reduce the MIC of an anti-microbial agent from the range in which the microbe is considered clinically resistant to a range in which the microbe is considered clinically susceptible. With this result, it is expected that concurrent administration in vivo of the ABC transporter inhibitor with the microbe will reverse resistance and effectively convert the microbe-resistant organism into a microbe-susceptible organism.

By measuring the effect of anti-microbial agents, such as antibiotics, on the in vitro growth curves of microbes, in the presence or absence of an inhibitor of an ABC drug transporter, the inhibitor of the ABC drug transporter may be shown to enhance the early anti-microbial effect of anti-microbial agents at 0-24 hours. Enhancement of early killing and/or growth inhibitory effects are important in determining therapeutic outcome.

A “microbial infection” is a pathological condition characterized by undesired growth of a microbe in or on a multicellular organism, particularly in or on animals and agricultural plants, most particularly in or on mammals, including humans. The terms “microbe” or “microbial cell” include all unicellular organisms such as bacteria, protozoan parasites, and unicellular fungi, i.e., yeasts.

The present invention relates to methods and materials for treating subjects suffering from microbial infections. The microbial infection may be a bacterial infection, such as a gram-positive bacterial infection or a gram-negative infection, a fungal infection, or a protozoal infection.

“Gram-positive bacterial infection,” as used herein, encompasses conditions associated with or resulting from gram-positive bacterial infection (e.g., sequelae). These conditions include gram-positive sepsis and one or more of the conditions associated therewith, including bacteremia, fever, hypotension, shock, metabolic acidosis, disseminated intravascular coagulation and related clotting disorders, anemia, thrombocytopenia, leukopenia, adult respiratory distress syndrome and related pulmonary disorders, renal failure and related renal disorders, hepatobiliary disease and central nervous system disorders. These conditions also include translocation of gram-negative bacteria from the intestines and concomitant release of endotoxin. Gram-positive bacteria include bacteria from the following species: Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, and Corynebacterium. A variety of gram-positive organisms are capable of causing sepsis. The most common organisms involved in sepsis are Staphylococcus aureus, Streptoccocus pneumoniae, coagulase-negative staphylococci, beta-hemolytic streptococci, and enterococci, but any gram-positive organism may be involved. Bone, J. Critical Care, 8: 51-59 (1993).

“Gram-negative bacterial infection,” as used herein, encompasses conditions associated with or resulting from gram-negative bacterial infection (e.g., sequelae). These conditions include gram-negative sepsis, endotoxin-related hypotension and shock, and one or more of the conditions associated therewith, including fever, metabolic acidosis, disseminated intravascular coagulation and related clotting disorders, anemia, thrombocytopenia, leukopenia, adult respiratory distress syndrome and related pulmonary disorders, renal failure and related renal disorders, hepatobiliary disease and central nervous system disorders. These conditions also include translocation of bacteria from the intestines and concomitant release of endotoxin. Gram-negative bacteria include bacteria from the following species: Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, and Yersinia species.

A “protozoal infection,” as used herein encompasses conditions associated with or resulting from a protozoal infection. Protozoan organisms include the following species: Toxoplasma including Toxoplasma gondii; Leishmania species; Trypanosoma species including Trypanosoma cruzi; Plasmodium species including Plasmodium vivax; Plasmodium falciparum, Plasmodium ovale and Plasmodium malariae.

A “fungal infection,” as used herein encompasses conditions associated with or resulting from a fungal infection. Fungal species include, but are not limited to: Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, and Saksenaea.

### Antibiotic Resistance

The term “drug resistance” refers to the circumstance when a disease does not respond to a treatment drug. Drug resistance can be either intrinsic or acquired. “Multidrug resistance” means a specific type of drug resistance characterized by cross-resistance of a disease to more than one functionally and/or structurally unrelated drugs. The term “ABC transporter-mediated multidrug resistance” refers to multidrug resistance due to the activity of an ABC drug transporter protein.

Anti-microbial agents, such as antibiotics, have been effective tools in the treatment of infectious diseases during the last half century. From the development of anti-microbial therapy to the late 1980s there was almost complete control over bacterial infections in developed countries. The emergence of resistant bacteria, especially during the late 1980s and early 1990s, is changing this situation. The increase in antibiotic resistant strains has been particularly common in major hospitals and care centers. The consequences of the increase in resistant strains include higher morbidity and mortality, longer patient hospitalization, and an increase in treatment costs. (See, e.g., B. Murray, 1994, New Engl. J. Med. 330: 1229-1230).

The constant use of anti-microbial agents, such as antibiotics in the hospital environment has selected microbial populations, including bacterial populations, that are resistant to many antibiotics. These populations include opportunistic pathogens that may not be strongly virulent but that are intrinsically resistant to a number of antibiotics. Such microbes often infect debilitated or immunocompromised patients. The emerging resistant populations also include strains of bacterial species that are well known pathogens, which previously were susceptible to antibiotics. The newly acquired resistance is generally due to DNA mutations, or to resistance plasmids (R plasmids) or resistance-conferring transposons transferred from another organism. Multidrug resistance, on the other hand, is commonly due to over expression of an ABC family member. For example, infections by either type of bacterial population, naturally resistant opportunistic pathogens or antibiotic-resistant pathogenic bacteria, are difficult to treat with current antibiotics. New antibiotic molecules which can override the mechanisms of resistance are needed.

Antibiotic resistance in bacteria is an increasingly troublesome problem. The accelerating development of antibiotic-resistant bacteria, intensified by the widespread use of antibiotics in farm animals and overprescription of antibiotics by physicians, has been accompanied by declining research into new antibiotics with different modes of action. Science, 264: 360-374 (1994). Antibiotic resistance, once acquired, can be rapidly spread to other bacteria, including bacteria of a different species. There are some species of bacteria that are resistant to all but one antibiotic; it may be only a matter of time before the appearance of bacterial strains that are resistant to all antibiotics.

Bacteria have developed several different mechanisms to overcome the action of antibiotics. These mechanisms of resistance can be specific for a molecule or a family of antibiotics, or can be non-specific and be involved in resistance to unrelated antibiotics. Several mechanisms of resistance can exist in a single bacterial strain, and those mechanisms may act independently or they may act synergistically to overcome the action of an antibiotic or a combination of antibiotics. Specific mechanisms include degradation of the drug, inactivation of the drug by enzymatic modification, and alteration of the drug target. (See, e.g., B. G. Spratt, Science 264:388 (1994)). There are, however, more general mechanisms of drug resistance, in which access of the antibiotic to the target is prevented or reduced by decreasing the transport of the antibiotic into the cell or by increasing the efflux of the drug from the cell to the outside medium. Both mechanisms can lower the concentration of drug at the target site and allow bacterial survival in the presence of one or more antibiotics which would otherwise inhibit or kill the bacterial cells. Some bacteria utilize both mechanisms, combining a low permeability of the cell wall (including membranes) with an active efflux of antibiotics. (See, e.g., H. Nikaido, Science 264:382-388 (1994)).

Some cases of microbial multidrug resistance are due to the action of efflux pumps. Once in the cytoplasm or periplasm a drug can be transported back to the outer medium. This transport is mediated by efflux pumps, which are constituted of proteins. Different pumps can efflux specifically a drug or group of drugs, such as the NorA system that transports quinolones, or Tet A that transports tetracyclines, or they can efflux a large variety of molecules, such as certain efflux pumps of Pseudomonas aeruginosa. In general, efflux pumps have a cytoplasmic component and energy is required to transport molecules out of the cell. Some efflux pumps have a second cytoplasmic membrane protein that extends into the periplasm. At least some efflux pumps of P. aeruginosa have a third protein located in the outer membrane.

Efflux pumps are involved in antibiotic resistance since, in some cases, they can remove a significant fraction of the antibiotic molecules which manage to enter the cells, thereby maintaining a very low intracellular antibiotic concentration. To illustrate, P. aeruginosa laboratory-derived mutant strain 799/61 which does not produce any measurable amounts of efflux pump is 8 to 10 fold more susceptible to tetracycline and ciprofloxacin than the parent strain P. aeruginosa 799, which synthesizes efflux pumps. Also, null mutants of mexA, the cytoplasmic component of a P. aeruginosa efflux pump, are more susceptible to antibiotics than the wild type.

The physiological role of efflux pumps has not been clearly defined yet. They are involved in drug resistance but they also are involved in the normal physiology of the bacterial cell. The efflux pump coded in the mexA operon of P. aeruginosa has been shown to be regulated by the iron content of the medium, and it is co-regulated with the synthesis of the receptors of siderophores. Siderophores are molecules that are needed for bacterial growth under iron starvation conditions, such as during infection of an animal. They are synthesized in the cytoplasm and exported when the bacterial cell needs iron. Siderophores scavenge iron within the infected animal and return the iron to the microbe to be used for essential microbial processes. Since there is essentially no free iron in the bodies of animals, including the human body, the production of siderophores by infecting bacteria is an important virulence factor for the progress of the infection.

The susceptibility of a bacterial species to an antibiotic is generally determined by two microbiological methods. A rapid but crude procedure uses commercially available filter paper disks that have been impregnated with a specific quantity of the antibiotic drug. These disks are placed on the surface of agar plates that have been streaked with a culture of the organism being tested, and the plates are observed for zones of growth inhibition. A more accurate technique, the broth dilution susceptibility test, involves preparing test tubes containing serial dilutions of the drug in liquid culture media, then inoculating the organism being tested into the tubes. The lowest concentration of drug that inhibits growth of the bacteria after a suitable period of incubation is reported as the minimum inhibitory concentration.

The resistance or susceptibility of an organism to an antibiotic is determined on the basis of clinical outcome, e.g., whether administration of that antibiotic to a subject infected by that organism will successfully cure the subject. While an organism may literally be susceptible to a high concentration of an antibiotic in vitro, the organism may in fact be resistant to that antibiotic at physiologically realistic concentrations. If the concentration of drug required to inhibit growth of or kill the organism is greater than the concentration that can safely be achieved without toxicity to the subject, the microorganism is considered to be resistant to the antibiotic. To facilitate the identification of antibiotic resistance or susceptibility using in vitro test results, the National Committee for Clinical Laboratory Standards (NCCLS) has formulated standards for antibiotic susceptibility that correlate clinical outcome to in vitro determinations of the minimum inhibitory concentration of antibiotic.

### Fungal Infections

Most endemic mycoses are acquired by the respiratory route and are minimally symptomatic; cough, fever, headache, and pleuritic pain may be seen. Occasionally, endemic mycoses may cause progressive pulmonary disease or systemic infection. Histoplasmosis, caused by Histoplasma, is the most common endemic respiratory mycosis in the United States; over 40 million people have been infected. The disease is noncontagious and ordinarily self-limited, but chronic pulmonary infection and disseminated infection may occur. Pulmonary infection rarely requires treatment, but disseminated infection may be treated with amphotericin B. Coccidioidomycosis, caused by Coccidioides, is a noncontagious respiratory mycosis prevalent in the southwest. It also is usually self-limited but may lead to chronic pulmonary infection or disseminated infection. Amphotericin B or miconazole may be given for treatment. Blastomycosis, caused by Blastomyces is a noncontagious, subacute or chronic endemic mycosis most commonly seen in the southeast. Most pulmonary infections are probably self-limited. Patients with progressive lung disease or disseminated disease, and immunocompromised patients, may be treated systemically with amphotericin B. Paracoccidioidomycosis, caused by Paracoccidioides, is a noncontagious respiratory mycosis that is the most common systemic mycosis in South America. It may be acute and self-limited or may produce progressive pulmonary disease or extrapulmonary dissemination. Disseminated disease is generally fatal in the absence of therapy. Sulfonamides may be used but have a low success rate. Amphotericin B produces a higher response rate but relapses may still occur.

Cryptococcosis is a noncontagious, often opportunistic mycosis. It is characterized by respiratory involvement or hematogenous dissemination, often with meningitis. A major etiologic agent is C. neoformans. Most pulmonary infections are probably overlooked, but cryptococcal meningitis, which accounts for 90% of reported disease, is dramatic and seldom overlooked. Cryptococcosis is a particular problem in immunocompromised patients; cryptococcal meningitis occurs in 7 to 10% of AIDS patients. The principal symptom of meningitis is headache; associated findings include mental changes, ocular symptoms, hearing deficits, nausea, vomiting, and seizures. Without treatment, 80% of patients die within two years. In meningitis, cryptococci can be observed in India ink preparations of cerebrospinal fluid sediment, and can be cultured from the cerebrospinal fluid. Treatment is generally with fluconazole or the combination of amphotericin B and flucytosine, although amphotericin B does not cross the blood brain barrier. Co-administration of an opioid drug transporter inhibitor with an anti-fungal agent may be particularly effective for the treatment of meningitis. Co-owned and co-pending U.S. patent application Ser. No. 10/000,113 (hereby incorporated by reference) describes how opioid drug transporter inhibitors can increase the effectiveness of therapeutic agents acting across the blood-brain barrier.

Aspergillosis is a term that encompasses a variety of disease processes caused by Aspergillus species. Aspergillus species are ubiquitous; their spores are constantly being inhaled. Of the more than 300 species known, only a few are ordinarily pathogenic for man: A. fumigatus, A. flavus, A. niger, A. nidulans, A. terreus, A. sydowi, A. flavatus, and A. glaucus. Aspergillosis is increasing in prevalence and is particularly a problem among patients with chronic respiratory disease or immunocompromised patients. Among immunocompromised patients, aspergillosis is second only to candidiasis as the most common opportunistic mycosis and accounts for about 15% of the systemic mycoses in this group. Opportunistic pulmonary aspergillosis is characterized by widespread bronchial erosion and ulceration, followed by invasion of the pulmonary vessels, with thrombosis, embolization and infarction. Clinically, infection manifests as a necrotizing patchy bronchopneumonia, sometimes with hemorrhagic pulmonary infarction. In about 40% of eases, there is hematogenous spread to other sites. Aspergillosis is also a rare but devastating complication of burn wounds; amputation is often required for cure. Invasive aspergillosis is commonly fatal, so aggressive diagnosis and treatment is required. Blood, urine and cerebrospinal fluid cultures are rarely positive, but fungi can be seen in smears and biopsies. Amphoteficin B can be given for treatment.

Mucormycosis is an acute suppurative opportunistic mycosis that produces rhinocerebral, pulmonary or disseminated disease in immunocompromised patients, and local or disseminated disease in patients with burns or open wounds. Infection is caused by fungi in the class Zygomycetes, and include Basidiobolus, Conidiobolus, Rhizopus, Mucor, Absidia, Mortierella, Cunninghamella, and Saksenaea. Rhinocerebral mucormycosis accounts for about half of all cases of mucormycosis. It is one of the most rapidly fatal fungal diseases, with death occurring within 2-10 days in untreated patients. Early clinical signs include nasal stuffiness, bloody nasal discharge, facial swelling and facial pain. The infection then spreads to the eyes, cranial nerves and brain. Thus, as with meningitis, mucormycosis may be a particularly suitable condition for the invention

Candidiasis is a general term for a variety of local and systemic processes caused by colonization or infection of the host by species of the yeast Candida. Candidiasis occurs worldwide; superficial infections of the skin, mouth and other mucus membranes are universal. Invasive systemic disease has become a problem due to the use of high doses of antibiotics that destroy normal bacterial flora, immunosuppressive agents, and agents toxic to bone marrow, e.g., during cancer therapy. Neutropenia is a major risk factor for Candida dissemination. Candidiasis is also seen among immunocompromised individuals such as AIDS patients, organ transplant patients, patients receiving parentera nutrition, and cancer patients undergoing radiation treatment and chemotherapy. It is the most common opportunistic mycosis in the world. The most common etiologic agent is Candida albicans. Other infectious species include C. tropicalis, C. parapsilosis, C stellatoidea, C. krusei, C. parakrusei, C. lusitanae, C. pseudotropicalis, C. guilliermondi and C. glabrata. Candida albicans is normally found in the mouth, throat, gastrointestinal tract and vagina of humans. Non-albicans species frequently colonize skin. Candida species occur in two forms that are not temperature- or host-dependent. The usual colonizing form are yeasts that may assume a pseudomycelial configuration, especially during tissue invasion. Pseudomyceliae result from the sequential budding of yeasts into branching chains of elongated organisms.

Most cases of systemic candidiasis are caused by Candida albicans and C. tropicalis, and increasingly, C. glabrata. Clinical manifestations of Candida infection appear mainly in the eyes, kidneys and skin. In the eyes, there may be single or multiple raised, white, fluffy chorioretinal lesions. These lesions are a potential cause of blindness. Involvement of the kidneys includes diffuse abscesses, capillary necrosis and obstruction of the ureters. Infection may result in progressive renal insufficiency. Systemic Candida infection can also manifest as maculonodular skin lesions surrounded by a reddened area; these lesions have an appearance similar to acne but are a major clue to a potentially lethal disease. Other manifestations of systemic candidiasis may include osteomyelitis, arthritis, meningitis, and abscesses in the brain, heart, liver, spleen and thyroid. Involvement of the lungs is also common, but pulmonary lesions are usually too small to be seen on chest X-ray. Finally, Candida endocarditis can occur in patients receiving prolonged intravenous therapy or cardiac valve implants, or in intravenous drug abusers. Fungal lesions appear on the valves, and can embolize and occlude large blood vessels.

Mucocutaneous infections may be treated with topical preparations of nystatin, amphotericin B, clotrimazole, miconazole, haloprogin or gentian violet. Oropharyngeal or esophageal candidiasis can be treated with systemic agents such as ketoconazole or fluconazole. Chronic mucocutaneous candidiasis syndrome may respond to topical or systemic therapeutic agents such as amphotericin B or ketoconazole, but often relapses when medication is discontinued. Cystitis may be treated with amphotericin B bladder rinses, or a brief low-dose intravenous course of amphotericin B with or without oral flucytosine. Endocarditis is essentially incurable without valve replacement, accompanied by a 6 to 10 week course of amphotericin B and flucytosine. Even with therapy, however, complete cure of endocarditis is not always possible.

The mortality rate from systemic candidiasis is about 50%. Systemic candidiasis may be treated with fluconazole, a fungistatic agent, or amphotericin B, a fungicidal agent although systemic use of the latter is limited by its toxicity. Both drugs have substantial adverse reactions when used in combination with cyclosporine A, which itself can be nephrotoxic. The removal of precipitating factors such as intravenous lines or catheters is also important for controlling infection. Flucytosine therapy can be added to the amphotericin B therapy for treatment of systemic candidiasis, especially in patients that are not immunocompromised. In immunocompromised patients, however, these infections are problematic and resist effective treatment. Mortality with systemic candidiasis can be over 90% in such patients. Furthermore, chronic mucocutaneous candidiasis and candidal endocarditis often show evidence of disease after having been declared cured.

There continues to exist a need in the art for improved antifungal methods and materials. In particular, effective antifungal therapy for systemic mycoses is limited. Products and methods responsive to this need would ideally involve substantially non-toxic compounds available in large quantities by means of synthetic or recombinant methods. Ideal compounds would have a rapid effect and a broad spectrum of fungicidal or fungistatic activity against a variety of different fungal species when administered or applied as the sole antifungal agent. Ideal compounds would also be useful in combinative therapies with other antifungal agents, particularly where these activities would reduce the amount of antifungal agent required for therapeutic effectiveness, enhance the effect of such agents, or limit potential toxic responses and high cost of treatment.

### Anti-microbial Agents

As used herein, the terms “anti-microbial agent” and “antibiotic” mean any therapeutic agent that suppresses the growth of microorganisms, such as bacteria, fungi (including yeasts), actinomycetes, and protozoan organisms. Antibiotics are natural chemical substances of relatively low molecular weight produced by various species of microorganisms, such as bacteria (including Bacillus species), actinomycetes (including Streptomyces) and fungi, that inhibit growth of or destroy other microorganisms. Substances of similar structure and mode of action may be synthesized chemically, or natural compounds may be modified to produce semi-synthetic antibiotics. These biosynthetic and semi-synthetic derivatives are also effective as antibiotics. The major classes of antibiotics are (1) the β-lactams, including the penicillins, cephalosporins and monobactams; (2) the aminoglycosides, e.g., gentamicin, tobramycin, netilmycin, and amikacin; (3) the tetracyclines; (4) the sulfonamides and trimethoprim; (5) the fluoroquinolones, e.g., ciprofloxacin, norfloxacin, and ofloxacin; (6) vancomycin; (7) the macrolides, which include for example, erythromycin, azithromycin, and clarithromycin; and (8) other antibiotics, e.g., the polymycins, chloramphenicol and the lincosamides.

Anti-microbial agents may achieve their therapeutic effects though several mechanisms that include (1) inhibiting synthesis of bacterial cell walls, including penicillins, cephalosporins, cycloserine, vancomycin, bacitracin, and the azole antifungal agents (e.g., clotrimazole, fluconazole and itraconazole); (2) acting directly on the cell membrane, affecting permeability and leading to leakage of intracellular compounds; these include, detergents, and the polyene antifungal agents, such as nystatin and amphotericin B; (3) affecting the function of 30S or 50S ribosomal subunits to cause irreversible inhihition of protein synthesis, including chloramphenicol, the tetracyclines, erythromycin, clindamycin, and pristamycins; (4) binding to the 30S ribosomal subunit and alter protein synthesis, these include the aminoglysides; (5) affecting bacterial nucleic acid metabolism, such as the rifamycins (e.g., rifampicin) and the quinolones; (6) anti-metabolites, including trimethaprim and the sulfonamides; and (7) nucleic acid analogues such as acyclovir, ganciclovir, zidovudine, or lamivudine. The class of anti-microbial agents are not limited to agents that act solely upon microbial species, compounds such as daunomycin and doxorubicin are useful both as anti-microbial agents as well as anti-tumor agents. Co-owned and co-pending U.S. patent application Ser. No. 10/______ (hereby incorporated by reference) describes how opioid drug transporter inhibitors can increase the effectiveness of the therapeutic agent acting across a cancer cell membrane.

Suitable antibiotics, and therapeutically effective concentrations thereof when administered with ABC drug transporter inhibitors, may be determined in in vivo models or according to in vitro tests, for example, in vitro minimum inhibitory concentration (MIC) and in vivo animal models, such as mouse peritonitis or rabbit bacteremia assays. Suitable antibiotics are antibiotics that are substrates of an ABC drug transporter and may act on the bacterial cell wall, cell membrane, protein metabolism or nucleic acid metabolism. These would include antibiotics or combinations of antibiotics from the following classes: β-lactam antibiotics with or without β-lactamase inhibitors, aminoglycosides, tetracyclines, sulfonamides and trimethoprim, vancomycin, macrolides, fluoroquinolones and quinolones, polymyxins, and other antibiotics. Dosage and administration of suitable antibiotics are known in the art, and briefly summarized below. The present invention contemplates the use of doses, including therapeutic and sub-therapeutic doses of the anti-microbial agent in combination with an opioid ABC drug transporter inhibitor. Thus one of skill in the art would understand that the summary below is simply a guideline and that dosages under the present invention may vary from the standard dosages provided.

### Pencillins

The penicillins have a characteristic double-ring system composed of a β-lactam ring, which provides the antibacterial activity, and a thiazolidene ring. The penicillins are differentiated by a single side chain that is unique for each penicillin. The compounds are bactericidal and act by inhibiting bacterial transpeptidase, an enzyme involved in synthesis of the bacterial cell wall. Because of their mechanism of action, penicillins are generally active against growing, but not resting, cells. Penicillins, especially penicillin G, have largely gram-positive activity; the relative insensitivity of gram-negative rods to penicillin G and several other penicillins is probably due to the permeability barrier of the outer membrane of gram-negative bacteria. Ampicillin, carbenicillin, ticarcillin, and some other penicillins are active against gram-negative bacteria because they can pass through this outer membrane. Penicillins have relatively few adverse effects, the most important of which are the hypersensitivity (allergic) reactions. These compounds are widely distributed in the body, but do not enter cells and do not usually accumulate in CSF.

One method of bacterial resistance to the penicillins is by production of the enzyme β-lactamase, which catalyzes hydrolysis of the β-lactam ring.

When an ABC drug transporter inhibitor is concurrently administered with a penicillin, for treatment of a bacterial infection, the penicillin is generally given in doses ranging from 1 μg/kg to 750 mg/kg daily, preferably not to exceed 24 grams daily for adults (or 600 mg/kg daily for children), and is preferably administered as follows:

Penicillin G is preferably administered parenterally to adults in doses ranging from 600,000 to 1,000,000 units per day. In conventional administration, it is effective largely against gram-positive organisms. For treatment of pneumococcal meningitis, penicillin G is administered in doses of 20-24 million units daily, in divided doses every 2 or 3 hours. For children, the preferred parenteral dose of penicillin G is 300,000 to 1,000,000 units per day. One unit of penicillin G contains 0.6 μg of pure sodium penicillin G (i.e., 1 mg is 1667 units).

Amoxicillin may be administered parenterally to adults in doses ranging from 750 mg to 1.5 grams per day, in 3 equally divided doses. For children, preferred parenteral doses of amoxicillin range from 20 to 40 mg/kg per day in 3 equally divided doses. Amoxicillin is also available in combination with clavulanic acid, a β-lactamase inhibitor. A 250 mg dose of the combination drug amoxicillin/clavulanate will contain 250 mg of amoxicillin and either 125 or 62.5 mg of clavulanic acid. The combination is preferably administered to adults orally in doses of 750 mg per day divided into 3 equal doses every 8 hours, with a preferred dose of 1.5 grams per day for severe infections, given in 3 equally divided doses. In children, the preferred oral dose is 20 to 40 mg/kg per day in 3 equally divided doses.

Ampicillin is preferably administered parenterally to adults in doses of 6 to 12 grams per day for severe infections, in 3 to 4 equally divided doses. In children, the preferred parenteral dose of ampicillin is 50 to 200 mg/kg per day in 3 to 4 equally divided doses. Larger doses of up to 400 mg/kg per day, for children, or 12 grams per day, for adults, may be administered parenterally for treatment of meningitis. Ampicillin is also available in combination with sulbactam, a β-lactamase inhibitor. Each 1.5 gram dose of ampicillin/sulbactam contains 1 gram of ampicillin and 0.5 grams of sulbactam. The combination is preferably administered parenterally to adults in doses of 6 to 12 grams per day divided into 4 equal doses every 6 hours, not to exceed a total of 12 grams per day.

Azlocillin is preferably administered parenterally to adults in doses of 8 to 18 grams per day, given in 4 to 6 equally divided doses.

Carbenicillin is preferably administered parenterally to adults in doses of 30 to 40 grams per day, given by continuous infusion or in 4 to 6 equally divided doses. Daily doses of up to 600 mg/kg have been used to treat children with life-threatening infections.

Mezlocillin is preferably administered to adults parenterally in doses of 100 to 300 mg/kg per day, given in 4 to 6 equally divided doses. The usual dose is 16 to 18 grams per day; for life threatening infections, 350 mg/kg per day may be administered, but in doses not to exceed 24 grams per day given in 6 equally divided doses every 4 hours. For children, the preferred parenteral dose of mezlocillin is 150 to 300 mg/kg per day.

Nafcillin is preferably administered intravenously to adults in doses of 3 grams per day, given in 6 equally divided doses every 4 hours, with doubled doses for very severe infections. In conventional administration, it is effective largely against gram-positive organisms. In children, the preferred parenteral dose is 20 to 50 mg/kg per day, in 2 equally divided doses every 12 hours. The preferred oral dose for nafcillin ranges from 1 gram per day to 6 grams per day in 4 to 6 divided doses.

Oxacillin is preferably administered parenterally to adults in doses of 2 to 12 grams per day, in 4 to 6 equally divided doses. In conventional administration, it is effective largely against gram-positive organisms. In children, oxacillin is preferably administered in doses of 100 to 300 mg/kg per day.

Piperacillin is preferably administered parenterally to adults in doses ranging from 100 mg/kg, or 6 grams per day, in 2 to 4 equally divided doses, up to a maximum of 24 grams per day, in 4 to 6 equally divided doses. Higher doses have been used without serious adverse effects.

Ticarcillin is preferably administered parenterally to adults in doses ranging from 4 grams per day to 18 grams per day administered in 4 to 6 equally divided doses. The usual dose is 200 to 300 mg/kg per day. For children, the preferred parenteral dose of ticarcillin ranges from 50 mg/kg per day to 300 mg/kg per day, given in 3, 4 or 6 equally divided doses. The combination ticarcillin/clavulanate is preferably administered parenterally to adults in doses of 200 to 300 mg/kg per day (based on ticarcillin content), in 4 to 6 equally divided doses. For adults, the usual dose is 3.1 grams (which contains 3 grams of ticarcillin and 100 mg of clavulanic acid) every 4 to 6 hours. The combination is also available in a dose of 3.2 grams, which contains 3 grams of ticarcillin and 200 mg of clavulanic acid.

In general, it is desirable to limit each intramuscular injection of a penicillin or cephalosporin to 2 grams; larger doses should be administered by multiple injections in different large muscle masses.

### Cephalosporins

The cephalosporins are characterized by a β-lactam ring, like the penicillins, but have an adjacent dihydrothiazine ring instead of a thiazolidene ring. For convenience, these compounds are generally classified by generations. The first generation includes cephalothin, cephapirin, cefazolin, cephalexin, cephradine and cefadroxil. These drugs generally have excellent gram-positive activity except for enterococci and methicillin-resistant staphylococci, and have only modest gram-negative coverage. The second generation includes cefamandole, cefoxitin, ceforanide, cefuroxime, cefuroxime axetil, cefaclor, cefonicid and cefotetan. This generation generally loses some gram-positive activity by weight and gains limited gram-negative coverage. The third generation includes cefotaxime, moxalactam, ceftizoxime, ceftriaxone, cefoperazone and ceftazidime. These compounds generally sacrifice further gram-positive activity by weight but gain substantial gram-negative coverage against Enterobacter and sometimes are active against Pseudoraonas. The cephalosporins bind to penicillin-binding proteins with varying affinity. Once binding occurs, protein synthesis is inhibited. Cephalosporins are usually well tolerated; adverse effects include hypersensitivity reactions and gastrointestinal effects. Cephalosporins may interact with nephrotoxic drugs, particularly aminoglycosides, to increase toxicity. Resistance to cephalosporins is mediated by several mechanisms, including production of β-lactamase, although some strains that do not produce β-lactamase are nevertheless resistant. ABC transporter proteins may mediate such resistance to cephalosporins.

When an ABC drug transporter inhibitor is concurrently administered with a cephalosporin, for treatment of a bacterial infection, the cephalosporin is generally given in doses ranging from 1 μg/kg to 500 mg/kg daily, preferably not to exceed 16 grams daily, and is preferably administered as follows:

Cefamandole is preferably administered parenterally to adults in doses ranging from 1.5 grams per day, given in 3 equally divided doses every 8 hours, to 12 grams per day for life-threatening infections, given in 6 equally divided doses every 4 hours. In children, cefamandole is preferably administered in doses ranging from 50 to 150 mg/kg per day, in 3 to 6 equally divided doses, not to exceed a total of 12 grams per day.

Cefazolin is preferably administered parenterally to adults in doses of 750 mg per day, given in 3 equally divided doses every 8 hours. In severe, life-threatening infections, it may be administered at doses of 6 grams per day divided into 4 equal doses every 6 hours; in rare instances, up to 12 grams per day have been used. In children, the preferred parenteral dose of cefazolin is 20 to 50 mg/kg per day, divided into 3 or 4 equal doses, with 100 mg/kg per day administered for severe infections.

Cefonicid is preferably administered parenterally to adults in doses ranging from 500 mg once daily, to 2 grams once daily for life-threatening infections. For intramuscular administration, a 2 gram dose should be divided into two 1-gram injections.

Cefoperazone is preferably administered parenterally to adults in doses ranging from 2 grams per day, given in 2 equally divided doses every 12 hours, to 12 grams per day for severe infections, given in 2, 3 or 4 equally divided doses. Doses up to 16 grams per day have been administered without complications.

Cefotetan is preferably administered parenterally to adults in doses of 1 to 4 grams per day, in 2 equally divided doses every 12 hours. Cefotetan may be administered in higher doses for fife-threatening infections, not to exceed a total dose of 6 grams per day.

Cefotaxime is preferably administered parenterally to adults in doses ranging from 1 to 12 grams per day, not to exceed 12 grams per day (2 grams every 4 hours) for fife-threatening infections. In children, the parenteral dose of cefotaxime is preferably 50 to 180 mg/kg, divided into 4 to 6 equal doses.

Cefoxitin is preferably administered parenterally to adults in doses ranging from 3 to 12 grams per day, given in 3, 4, or 6 equally divided doses. In children, cefoxitin is preferably administered parenterally in doses of 80 to 160 mg/kg per day, given in 4 or 6 equally divided doses, not to exceed a total dose of 12 grams per day.

Ceftazidime is preferably administered parenterally to adults in doses ranging from 500 mg per day, given in 2 to 3 equally divided doses (every 8 or 12 hours), up to a maximum of 6 grams per day. In children, ceftazidime is preferably administered intravenously in doses of 30 to 50 mg/kg, to a maximum of 6 grams per day.

Ceftizoxime is preferably administered parenterally to adults in doses ranging from 1 gram per day, given in 2 equally divided doses every 12 hours, to 12 grams per day for life-threatening infections, given in 3 equally divided doses every 8 hours. The usual adult dose is 1 to 2 grams every 8 or 12 hours. For children, the preferred parenteral dose is 50 mg/kg every 6 or 8 hours, for a total daily dose of 200 mg/kg.

Ceftriaxone is preferably administered parenterally to adults in doses ranging from 1 to 2 grams per day, given in 2 equally divided doses every 12 hours. It may be given in higher doses, not to exceed a total of 4 grams per day. In children, the preferred parenteral dose of ceftriaxone is 50 to 75 mg/kg per day, not to exceed 2 grams per day. In meningitis, ceftriaxone may be administered in doses of 100 mg/kg per day, not to exceed 4 grams per day.

Cefuroxime is preferably administered parenterally to adults in doses ranging from 2.25 to 4.5 grams per day, in 3 equally divided doses every 8 hours. For life-threatening infections, 6 grams per day may be administered in 4 equally divided doses every 6 hours, and for meningitis, 9 grams per day may be administered in 3 equally divided doses every 8 hours. For children, the preferred parenteral dose of cefuroxime is 50 to 150 mg/kg per day in 3 to 4 equally divided doses, or 240 mg/kg per day for meningitis.

Cephalexin is formulated for oral administration, and is preferably administered orally to adults in doses ranging from 1 to 4 grams per day in 2 to 4 equally divided doses. For children, the preferred dose is 20 to 50 mg/kg per day in divided doses, with doses being doubled for severe infections.

Cephalothin is usually administered parenterally to adults in doses of 8 to 12 grams per day.

### Other Beta-lactams

Imipenem is a N-formimidoyl derivative of the mold product thienamycin. It contains a β-lactam ring and somewhat resembles penicillin except for differences in the second ring. It has activity against both gram-positive and gram-negative organisms and is resistant to most β-lactamases, although not those from Pseudomonas. It is marketed in combination with cilastin, a compound that inhibits inactivation of imipenem in the kidney by renal dihydropeptidase I enzyme. Cilastin increases the concentration of imipenem in urine, although not in blood.

When an ABC drug transporter inhibitor is concurrently administered with an imipenem antibiotic, for treatment of a bacterial infection, the imipenem is generally given in doses ranging from 1 μg/kg to 100 mg/kg daily, and is preferably administered as follows:

Imipenem is available in combination with cilastin, an inhibitor of the renal dipeptidase enzyme that rapidly inactivates imipenem. The combination is preferably administered intramuscularly to adults in doses of 1 to 1.5 grams per day, given in 2 equally divided doses every 12 hours. Intramuscular doses exceeding 1.5 grams per day are not recommended. The combination is preferably administered intravenously in doses ranging from 1 to 4 grams per day, in 4 equally divided doses every 6 hours; doses exceeding 50 mg/kg per day, or 4 grams per day, are not recommended.

Aztreonam is the first of a new group of antibiotics referred to as the monobactams. These agents have a β-lactam ring but lack the second ring characteristic of the penicillins and cephalosporins. It acts by binding to penicillin-binding proteins, and produces long, filamentous bacterial shapes that eventually lyse. Aztreonam is active only against aerobic gram-negative bacteria, is susceptible to inactivation by some β-lactamases, and has few adverse effects.

When a ABC drug transporter inhibitor is concurrently administered with a monobactam antibiotic, for treatment of a bacterial infection, the monobactam is generally given in doses ranging from 1 μg/kg to 200 mg/kg daily, and is preferably administered as follows:

Aztreonam is preferably administered parenterally to adults in doses ranging from 1 gram per day, given in 2 equally divided doses every 12 hours, up to a maximum recommended dose of 8 grams per day in cases of life-threatening infection, given in 3 or 4 equally divided doses.

### Aminoglycosides

The aminoglycosides contain amino sugars linked to an aminocyclitol ring by glycosidic bonds. They have similar mechanisms of action and properties, but differ somewhat in spectrum of action, toxicity, and susceptibility to bacterial resistance. The compounds are bactericidal, with activity against both gram-positive and gram-negative organisms, and act by binding to proteins on the 30S ribosome of bacteria and inhibiting protein synthesis. The aminoglycosides also bind to isolated LPS and have a very weak outer membrane permeabilizing effect. See, e.g., Taber et at., Microbiological Reviews 53: 439-457 (1987)); Kadurugamuwa et al., Antmicrobial Agents and Chemotherapy, 37: 715-721 (1993); Vaara, Microbiological Reviews 56: 395-411 (1992). This class of antibiotics includes amikacin, gentamicin, kanamycin, neomycin, netilmycin, paromomycin and tobramycin. The aminoglycosides are usually reserved for more serious infections because of severe adverse effects including ototoxicity and nephrotoxicity. There is a narrow therapeutic window between the concentration required to produce a therapeutic effect, e.g., 8 μg/mL for gentamicin, and the concentration that produces a toxic effect, e.g., 12 μg/mL for gentamicin. Neomycin in particular is highly toxic and is never administered parenterally.

When an ABC drug transporter inhibitor is concurrently administered with an aminoglycoside, for treatment of a bacterial infection, the aminoglycoside is generally given in doses ranging from 1 μg/kg to 20 mg/kg daily, preferably not to exceed 15 mg/kg daily, and is preferably administered as follows:

When administering aminoglycosides, it is desirable to measure serum peak and trough concentrations to ensure the adequacy and safety of the dosage. Dosages should generally be adjusted to avoid toxic peak and trough concentrations. Amikacin is preferably administered parenterally to adults and children in doses of 15 mg/kg per day, divided into two or three equal doses every 8 or 12 hours, and not to exceed a total dose of 1.5 grams per day. For uncomplicated infections, a dose of 500 mg amikacin per day, in 2 equally divided doses, may be administered. Dosages should be adjusted to avoid prolonged serum peak concentrations of amikacin above 35 μg/mL and prolonged trough concentrations greater than 10 μg/mL.

Gentamicin is preferably administered parenterally to adults in doses of 3 mg/kg per day, in three equally divided doses every 8 hours. For life-threatening infections, up to 5 mg/kg per day in 3 to 4 equally divided doses may be administered, but this dosage should be reduced to 3 mg/kg per day as soon as clinically indicated. For children, gentamicin is preferably administered parenterally in doses of 6 to 7.5 mg/kg per day. Dosages should be adjusted to avoid prolonged serum peak concentrations of gentamicin above 12 μg/mL and prolonged trough concentrations greater than 2 μg/mL.

Netilmicin may be administered parenterally to adults in doses ranging from 3 mg/kg per day, in 2 equally divided doses every 12 hours, to 6.5 mg/kg per day for serious systemic infection, in 2 or 3 equally divided doses. In children, the preferred parenteral dose is 5.5 to 8 mg/kg per day, in 2 or 3 equally divided doses. Dosages should be adjusted to avoid prolonged serum peak concentrations of netilmicin above 16 μg/mL and prolonged serum trough concentrations above 4 μg/mL.

Tobramycin is preferably administered parenterally to adults in doses of 3 mg/kg per day, given in three equally divided doses every 8 hours. For life-threatening infections, tobramycin may be administered in doses up to 5 mg/kg per day, in 3 or 4 equally divided doses, but this dosage should be reduced to 3 mg/kg per day as soon as clinically indicated. In children, tobramycin is preferably administered parenterally in doses of 6 to 7.5 mg/kg per day. Prolonged serum concentrations of tobramycin above 12 μg/mL should be avoided, and rising trough levels above 2 pg/mL may indicate tissue accumulation, which may contribute to toxicity.

Concurrent administration of ABC drug transporter inhibitor with the aminoglycosides, including amikacin, gentamicin, netilmicin and tobramycin, may permit a lowering of the dose of these toxic antibiotics necessary to achieve a therapeutic effect.

The present invention may be particularly suited to use with aminoglycoside anti-microbial agents. The present invention may permit lower and/or less frequent doses of the aminoglycoside to be administered to the patient. Further, the present invention may decrease the extreme side effects of such anti-microbial agents.

### Tetracyclines

Tetracyclines have a common four-ring structure and are closely congeneric derivatives of the polycyclic naphthacenecarboxamide. The compounds are bacteriostatic, and inhibit protein synthesis by binding to the 30S subunit of microbial ribosomes and interfering with attachment of aminoacyl tRNA. The compounds have some activity against both gram-positive and gram-negative bacteria; however, their use is limited because many species are now relatively resistant. Adverse effects include gastrointestinal effects, hepatotoxicity with large doses, and nephrotoxicity in some patients. This antibiotic class includes tetracycline, chlortetracycline, demeclocycline, doxycycline, methacycline, minocycline and oxytetracycline.

Tetracyclines probably penetrate bacterial cells by passive diffusion and inhibit bacterial growth by interfering with protein synthesis or by destroying the membrane. A growing number of various bacterial species acquire resistance to the bacteriostatic activity of tetracycline. The two widespread mechanisms of bacterial resistance do not destroy tetracycline: one is mediated by efflux pumps, the other involves an EF-G-like protein that confers ribosome protection. Several efflux transporters, including multidrug-resistance pumps and tetracycline-specific exporters, confer bacterial resistance against tetracycline. See, e.g., Schnappinger and Hillen, Tetracyclines: antibiotic action, uptake, and resistance mechanisms, Arch Microbiol 165:359-69 (1996).

When an ABC drug transporter inhibitor is concurrently administered with a tetracycline, for treatment of a bacterial infection, the tetracycline is generally given in doses ranging from 1 μg/kg to 50 mg/kg daily, and is preferably administered as follows:

The tetracycline antibiotics are generally administered to adults in doses of 1 to 2 grams per day. An exception is doxycycline, which is preferably administered intravenously to adults in doses of 100 to 200 mg per day, and to children in doses of 2 mg/lb per day. Tetracycline may be administered parenterally to adults in doses of 0.5 to 2 grams per day, in 2 equally divided doses, and to children in doses of 10 to 20 mg/kg per day.

### Sulfonamides

The sulfonamides are derivatives of sulfanilamide, a compound similar in structure to para-aminobenzoic acid (PABA), which is an essential precursor for bacterial synthesis of folic acid. The compounds are generally bacteriostatic, and act by competitively inhibiting incorporation of PABA into tetrahydrofolic acid, which is a required cofactor in the synthesis of thymidines, purines and DNA. Sulfonamides have a wide range of activity against gram-positive and gram-negative bacteria, but their usefulness has diminished with increasingly high prevalence of bacterial resistance. The sulfonamide class of antibiotics includes sulfacytine, sulfadiazine, sulfamethizole, sulfisoxazole, sulfamethoxazole, sulfabenzamide and sulfacetamide. Adverse effects include hypersensitivity reactions and occasional hematological toxicity.

Trimethoprim is an inhibitor of the dihydrofolate reductase enzyme, which converts dihydrofolic to tetrahydrofolic acid, a required factor for DNA synthesis. Adverse effects include gastrointestinal distress and rare hematological toxicity. Trimethoprim is also available in combination with sulfamethoxazole (also known as co-trimoxazole). The combination is usually bactericidal, although each agent singly is usually bacteriostatic. The combination is the drug of choice for Salmonella infections, some Shigella infections, E. coli traveler's diarrhea and Pneumocystis carinii pneumonia.

When an ABC drug transporter inhibitor is concurrently administered with a sulfonamide or trimethoprim, for treatment of a bacterial infection, the sulfonamide or trimethoprim is generally given in doses ranging from 1 μg/kg to 150 mg/kg daily, preferably not to exceed a combination dose of 960 mg trimethoprim/4.8 g sulfamethoxazole daily, and is preferably administered as follows:

The combination trimethoprinm/sulfamethoxazole is available in a formulation containing a 1:5 ratio of trimethoprim and sulfamethoxazole (e.g., 16 mg trimethoprim and 80 mg sulfamethoxazole). The combination is preferably administered intravenously to adults or children in doses of 8 to 10 mg/kg (based on the weight of the trimethoprim component) per day, in 2 to 4 equally divided doses. For Pneumocystis carinii infection, the combination can be administered in doses of 20 mg/kg (based on the weight of the trimethoprim component) per day, in 3-4 equally divided doses, to a maximum recommended dose of 960 mg trimethoprim/4.8 g sulfamethoxazole per day. Trimethoprim alone is preferably administered orally to adults in doses of 200 mg per day. Sulfamethoxazole alone is preferably administered orally to adults in doses of 2 to 3 grams per day, and to children orally in doses of 50 to 60 mg/kg per day.

### Fluoroquinolones

The fluoroquinolones and quinolones are derivatives of nalidixic acid, a naphthyridine derivative. These compounds are bactericidal, and impair DNA replication, transcription and repair by binding to the DNA and interfering with DNA gyrase, an enzyme which catalyzes negative supercoiling of DNA. The fluoroquinolones, which include norfloxacin, ciprofloxacin, and ofloxacin, and the quinolones, which include cinoxacin, have a broad spectrum of anti-microbial activity against gram-negative and gram-positive organisms. These compounds distribute widely through extravascular tissue sites, have a long serum half-life, and present few adverse effects. Because of their effect on DNA, the drugs are contraindicated in pregnant patients and in children whose skeletal growth is incomplete.

When an ABC drug transporter inhibitor is concurrently administered with a fluoroquinolone or quinolone, for treatment of a bacterial infection, the fluoroquinolone or quinolone is generally given in doses ranging from 1 μg/kg to 50 mg/kg daily, preferably not to exceed 1 gram daily, and is preferably administered as follows:

Norfloxacin is preferably administered orally to adults in doses from 400 to 800 mg daily, divided into two doses every 12 hours. Cinoxacin is preferably administered orally to adults in doses of 1 gram per day, given in 2 or 4 equally divided doses. Ciprofloxacin is preferably administered to adults intravenously in doses from 400 to 800 mg daily, or orally in doses from 500 to 1500 mg daily, divided into two doses every 12 hours. Ofloxacin is preferably administered to adults intravenously in doses from 400 to 800 mg daily, or orally in doses from 400 to 800 mg daily, divided into two doses every 12 hours.

### Vancomycin

Vancomycin is a glycopeptide, with a molecular weight of about 1500, produced by a fungus. It is primarily active against gram-positive bacteria. The drug inhibits one of the final steps in synthesis of the bacterial cell wall, and is thus effective only against growing organisms. It is used to treat serious infections due to gram-positive cocci when penicillin G is not useful because of bacterial resistance or patient allergies. Vancomycin has two major adverse effects, ototoxicity and nephrotoxicity. These toxicities can be potentiated by concurrent administration of another drug with the same adverse effect, such as an aminoglycoside.

When an ABC drug transporter inhibitor is concurrently administered with vancomycin, for treatment of a bacterial infection, the vancomycin is generally given in doses ranging from 1 mg/kg to 50 mg/kg daily, and is preferably administered parenterally to adults in doses of 2 grams per day, divided into 2 or 4 doses every 6 or 12 hours. In children it is preferably administered in doses of 40 mg/kg, given in 4 equally divided doses every 6 hours. In conventional administration, vancomycin is effective largely against gram-positive organisms.

### Macrolides

The macrolides are bacteriostatic and act by binding to the 50S subunit of 70S ribosomes, resulting in inhibition of protein synthesis. They have a broad spectrum of activity against gram-positive and bacteria and may be bacteriostatic or bactericidal, depending on the concentration achieved at sites of infection. The compounds distribute widely in body fluids. Adverse effects include gastrointestinal distress and rare hypersensitivity reactions. The most common macrolide used is erythromycin, but the class includes other compounds such as clarithromycin and azithromycin.

Example __ provides the unexpected result that erythromycin is an ABC transporter protein substrate. Therefore, the present invention explicitly contemplates co-administration of macrolides, such as erythromycin, with opioid ABC drug transporter inhibitors.

When an ABC drug transporter inhibitor is concurrently administered with a macrolide, for treatment of a bacterial infection, the macrolide is generally given in doses ranging from 1 μg/kg to 100 mg/kg daily, and is preferably administered as follows:

Erythromycin is preferably administered intravenously to adults and children in doses of 15 to 20 mg/kg per day, given by continuous infusion or in 4 equally divided doses every 6 hours. Erythromycin can be administered at doses up to 4 grams per day in cases of very severe infection.

Clarithromycin is preferably administered orally to adults in doses of 500 mg to 1 gram daily, in equally divided doses every 12 hours.

Azithromycin is preferably administered orally to adults at a dose of 500 mg on the first day of treatment followed by 250 mg once daily for 4 days, for a total dose of 1.5 grams.

### Other Antibiotics

The polymyxins are a group of closely related antibiotic substances produced by strains of Bacillus polymyxa. These drugs, which are cationic detergents, are relatively simple, basic peptides with molecular weights of about 1000. Their anti-microbial activity is restricted to gram-negative bacteria. They interact strongly with phospholipids and act by penetrating into and disrupting the structure of cell membranes. Polymyxin B also binds to the lipid A portion of endotoxin and neutralizes the toxic effects of this molecule. Polymyxin B has severe adverse effects, including nephrotoxicity and neurotoxicity, and should not be administered concurrently with other nephrotoxic or neurotoxic drugs. The drug thus has limited use as a therapeutic agent because of high systemic toxicity, but may be used for severe infections, such as P. aeruginosa meningitis, that respond poorly to other antibiotics.

Polymyxin B is generally given in doses ranging from 1 unit/kg to 45,000 units/kg daily, and is preferably administered intravenously to adults and children in doses of 15,000 to 25,000 units/kg per day, divided into 2 equal doses every 12 hours. It may be administered intramuscularly in doses of 25,000 to 30,000 units/kg per day, although these injections are very painful. Doses of polymyxin B as high as 45,000 units/kg per day have been used in limited clinical studies to treat neonates for Pseudomonas aeruginosa sepsis. Polymyxin B is the treatment of choice for P. aeruginosa meningitis, and is preferably administered intrathecally to adults and older children in doses of 50,000 units once daily for to 4 days, followed by 50,000 units every other day; in children under two years old, it is administered intrathecally in doses of 20,000 daily for 3 to 4 days, followed by 25,000 units every other day.

Chloramphenicol inhibits protein synthesis by binding to the 50S ribosomal subunit and preventing binding of aminoacyl tRNA. It has a fairly wide spectrum of anti-microbial activity, but is only reserved for serious infections, such as meningitis, typhus, typhoid fever, and Rocky Mountain spotted fever, because of its severe and fatal adverse hematological effects. It is primarily bacteriostatic, although it may be bactericidal to certain species.

Chloramphenicol is preferably administered intravenously to adults in doses of 50 mg/kg per day, in 4 equally divided doses; in exceptional cases, it can be administered in doses up to 100 mg/kg per day. In children, chloramphenicol is preferably administered intravenously in doses of 25 mg/kg per day, although up to 100 mg/kg per day can be administered in cases of severe infection.

Lincomycin and clindamycin are lincosamide anti-microbials. They consist of an amino acid linked to an amino sugar. Both inhibit protein synthesis by binding to the 50S ribosomal subunit. They compete with erythromycin and chloramphenicol for the same binding site but in an overlapping fashion. They may be bacteriostatic or bactericidal, depending on relative concentration and susceptibility. Gastrointestinal distress is the most common side effect. Other adverse reactions include cutaneous hypersensitivity, transient hematological abnormalities, and minor elevations of hepatic enzymes. Clindamycin is often the drug of choice for infections caused by anaerobic bacteria or mixed aerobic/anaerobic infections, and can also be used for susceptible aerobic gram-positive cocci.

Clindamycin is preferably administered parenterally to adults in doses ranging from 600 mg to 4.8 grams per day, given in 2, 3 or 4 equally divided doses. It is recommended that the dose in each intramuscular injection not exceed 600 mg. For children, clindamycin is preferably administered parenterally in doses of 15-40 mg/kg per day, given in 3 or 4 equally divided doses.

Dosages of all anti-microbial agents should be adjusted in patients with renal impairment or hepatic insufficiency, due to the reduced metabolism and/or excretion of the drugs in patients with these conditions. Doses in children should also be reduced, generally according to body weight. Those skilled in the art can readily optimize effective dosages and administration regimens for the ABC drug transporter inhibitor and the antibiotics in concurrent administration.

Some drugs, e.g. aminoglycosides, have a small therapeutic window. For example, 2 to 4 μg/mL of gentamicin or tobramycin may be required for inhibition of bacterial growth, but peak concentrations in plasma above 6 to 10 μg/mL may result in ototoxicity or nephrotoxicity. These agents are more difficult to administer because the ratio of toxic to therapeutic concentrations is very low. Anti-microbial agents that have toxic effects on the kidneys and that are also eliminated primarily by the kidneys, such as the aminoglycosides or vancomycin, require particular caution because reduced elimination can lead to increased plasma concentrations, which in turn may cause increased toxicity. Doses of anti-microbial agents that are eliminated by the kidneys must be reduced in patients with impaired renal function. Similarly, dosages of drugs that are metabolized or excreted by the liver, such as erythromycin, chloramphenicol, or clindamycin, must be reduced in patients with decreased hepatic function.

### Antifungal Agents

An ABC drug transporter inhibitor may be administered in conjunction with antifungal agents that are substrates for ABC transporters and are presently known to be effective. A preferred antifungal agent for this purpose is fluconazole. Concurrent administration of ABC drug transporter inhibitor with antifungal agents is expected to improve the therapeutic effectiveness of the antifungal agents. This may occur through reducing the amount of antifungal agent administered to a patient in order to eradicate or inhibit fungal growth. Because the use of some agents is limited by their systemic toxicity or prohibitive cost, lowering the concentration of antifungal agent required for therapeutic effectiveness reduces toxicity and/or cost of treatment, and thus allows wider use of the agent. Concurrent administration of ABC drug transporter inhibitor and an antifungal agent may produce a more rapid or complete fungicidal/fungistatic effect than could be achieved with the antifungal agent alone. ABC drug transporter inhibitor administration may reverse the resistance of fungi to antifungal agents. ABC drug transporter inhibitor administration may also convert a fungistatic agent into a fungicidal agent.

An advantage provided by the present invention is the ability to treat fungal infections, particularly Candida infections, that are presently considered incurable. Another advantage is the ability to treat fungi that have acquired resistance to known antifungal agents. A further advantage of concurrent administration of an ABC drug transporter inhibitor with an antifungal agent having undesirable side effects, e.g., amphotericin B, is the ability to reduce the amount of antifungal agent needed for effective therapy. The present invention may also provide quality of life benefits due to, e.g., decreased duration of therapy, reduced stay in intensive care units or reduced stay overall in the hospital, with the concomitant reduced risk of serious nosocomial (hospital-acquired) infections. Anti-fungal agents include three main groups.

The major group includes polyene derivatives, including amphotericin B and the structurally related compounds nystatin and pimaricin. These are broad-spectrum antifungals that bind to ergosterol, a component of fungal cell membranes, and thereby disrupt the membranes. Amphotericin B is usually effective for systemic mycoses, but its administration is limited by toxic effects that include fever and kidney damage, and other accompanying side effects such as anemia, low blood pressure, headache, nausea, vomiting and phlebitis. The unrelated antifungal agent flucytosine (5-fluorocytosine), an orally absorbed drug, is frequently used as an adjunct to amphotericin B treatment for some forms of candidiasis and cryptococcal meningitis. Its adverse effects include bone marrow depression with leukopenia and thrombocytopenia.

The second major group of antifungal agents includes azole derivatives which impair synthesis of ergosterol and lead to accumulation of metabolites that disrupt the function of fungal membrane-bound enzyme systems (e.g., cytochrome P450) and inhibit fungal growth. Significant inhibition of mammalian P450 results in significant drug interactions. This group of agents includes ketoconazole, clotrimazole, miconazole, econazole, butoconazole, oxiconazole, sulconazole, terconazole, fluconazole and itraconazole. These agents may be administered to treat systemic mycoses. Ketoconazole, an orally administered imidazole, is used to treat nonmeningeal blastomycosis, histoplasmosis, coccidioidomycosis and paracoccidioidomycosis in non-immunocompromised patients, and is also useful for oral and esophageal candidiasis. Adverse effects include rare drug-induced hepatitis; ketoconazole is also contraindicated in pregnancy. Itraconazole appears to have fewer side effects than ketoconazole and is used for most of the same indications. Fluconazole also has fewer side effects than ketoconazole that is used for oral and esophageal candidiasis and cryptococcal meningitis. Miconazole is a parenteral imidazole with efficacy in coccidioidomycosis and several other mycoses, but has side effects including hyperlipidemia and hyponatremia.

The third major group of antifungal agents includes allylaminesthiocarbamates, which are generally used to treat skin infections. This group includes tolnaftate and naftifine.

Another antifungal agent is griseofulvin, a fungistatic agent which is administered orally for fungal infections of skin, hair or nails that do not respond to topical treatment.

For administration to human subjects or in the treatment of any clinical conditions, The pharmaceutical compositions, doses or dosage forms of this invention may be utilized in compositions such as capsules, tablets or pills for oral administration, suppositories for rectal administration, liquid compositions for parenteral administration and the like. One or more doses may be administered according to methods of the invention.

The pharmaceutical compositions, doses or dosage forms of this invention may be used in the form of a pharmaceutical preparation, for example, in solid or semisolid form, which contains one or more of the drug transporter inhibitors, as an active ingredient, alone, or in combination with one or more therapeutic agents. Any drug transporter inhibitor or therapeutic agent, according to the invention may be in admixture with an organic or inorganic carrier or excipient suitable for external, enteral or parenteral applications. A drug transporter inhibitor may be compounded, for example, with the usual non-toxic, pharmaceutically acceptable carriers for capsules, tablets, pellets, suppositories, and any other form suitable for use. The carriers which can be used are water, glucose, lactose, gum acacia, gelatin, mannitol, starch paste, magnesium, trisilicate, talc, corn starch, keratin, colloidal silica, potato starch, urea and other carriers suitable for use in manufacturing preparations, in solid or semisolid form, and in addition auxiliary, stabilizing, thickening and coloring agents and perfumes may be used. A drug transporter inhibitor, alone or in conjunction with a therapeutic agent, is included in a pharmaceutical composition, dose, or dosage form in an amount sufficient to produce the desired effect upon the process or condition, including a variety of conditions and diseases in humans.

For preparing solid compositions such as tablets, the drug transporter inhibitor, alone or in conjunction with therapeutic agent, is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that a drug transporter inhibitor, alone or in conjunction with therapeutic agent, is dispersed evenly throughout the composition so that the composition may be readily subdivided into equally effective unit dosage forms such as capsules, tablets, caplets, or pills. The capsules, tablets, caplets, or pills of the novel pharmaceutical composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Controlled release (e.g., slow-release or sustained-release) dosage forms, as well as immediate release dosage forms are specifically contemplated according to the present invention.

Compositions in liquid forms in which a therapeutic agent may be incorporated for administration orally or by injection include aqueous solution, suitable flavored syrups, aqueous or oil suspensions, and emulsions with acceptable oils such as cottonseed oil, sesame oil, coconut oil or peanut oil, or with a solubilizing or emulsifying agent suitable for intravenous use, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Compositions for inhalation or insufflation include solutions and suspensions in pharmaceutically acceptable, aqueous or organic solvents, or mixtures thereof, and powders. The liquid or solid compositions may contain suitable pharmaceutical 1v acceptable excipients as set out above. Preferably, compositions are administered by the oral or nasal respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug transporter inhibitor alone, or in combination with a therapeutic agent, may be administered to the human subject by known procedures including but not limited to oral, sublingual, intramuscular, subcutaneous, intravenous, intratracheal, transmucosal, or transdermal modes of administration. When a combination of these compounds are administered, they may be administered together in the same composition, or may be administered in separate compositions. If a therapeutic agent and a drug transporter inhibitor are administered in separate compositions, they may be administered by similar or different modes of administration, or may be administered simultaneously with one another, or shortly before or after the other.

Drug transporter inhibitors alone, or in combination with therapeutic agents are formulated in compositions with a pharmaceutically acceptable carrier (“pharmaceutical compositions”). The carrier must be “acceptable” in the sense of being compatible with the other ingredients of the formulation and not deleterious to the recipient thereof. Examples of suitable pharmaceutical carriers include lactose, sucrose, starch, talc, magnesium stearate, crystalline cellulose, methyl cellulose, carboxymethyl cellulose, glycerin, sodium alginate, gum arabic, powders, saline, water, among others. The formulations may conveniently be presented in unit dosage and may be prepared by methods well-known in the pharmaceutical art, by bringing an active compound into association with a carrier or diluent, or optionally with one or more accessory ingredients, e.g., buffers, flavoring agents, surface active agents, or the like. The choice of carrier will depend upon the route of administration. Pharmaceutical compositions may be administered as solid or semisolid formulations, including as capsules, tablets, caplets, pills or patches. Formulations may be presented as an immediate release or as a controlled release (e.g., slow release or sustained release) formulation.

For oral or sublingual administration, a formulation may be presented as capsules, tablets, caplets, powders, granules or a suspension, with conventional additives such as lactose, mannitol, corn starch or potato starch; with binders such as crystalline cellulose, cellulose derivatives, acacia, corn starch, gelatins, natural sugars such as glucose or beta-lactose, corn sweeteners, natural and synthetic gums such as acacia, tragacanth, or sodium alginate, carboxymethylcellulose, polyethylene glycol, waxes, or the like; with disintegrators such as corn starch, potato starch, methyl cellulose, agar, bentonite, xanthan gums, sodium carboxymethyl-cellulose or the like; or with lubricants such as talc, sodium oleate, sodium stearate, magnesium stearate, sodium benzoate, sodium acetate, sodium chloride or the like.

For transdermal administration, a compound may be combined with skin penetration enhancers such as propylene glycol, polyethylene glycol, isopropanol, ethanol, oleic acid, N-methylpyrrolidone, or the like, which increase the permeability of the skin to the compounds, and permit the compounds to penetrate through the skin and into the bloodstream. Compound/enhancer compositions also may be combined additionally with a polymeric substance such as ethylcellulose, hydroxypropyl cellulose, ethylene/vinylacetate, polyvinyl pyrrolidone, or the like, to provide the composition in gel form, which can be dissolved in solvent such as methylene chloride, evaporated to the desired viscosity, and then applied to backing material to provide a patch.

For intravenous, intramuscular, or subcutaneous administration, compounds may combined with a sterile aqueous solution which is preferably isotonic with the blood of the recipient. Such formulations may be prepared by dissolving solid active ingredient in water containing physiologically compatible substances such as sodium chloride, glycine, or the like, and/or having a buffered pH compatible with physiological conditions to produce an aqueous solution, and/or rendering said solution sterile. Formulations may be present in unit or multi-dose containers such as sealed ampoules or vials.

When a drug transporter inhibitor is used in combination with the anti-microbial agent, the amount of the anti-microbial agent administered may be a therapeutic or sub-therapeutic amount. As used herein, a “therapeutic” amount is the amount of a therapeutic agent which causes a therapeutic effect in a subject administered the therapeutic agent alone. The amount of the drug transporter inhibitor may be an amount sufficient to reduce efflux of the anti-microbial agent from the microbe, increase the intracellular concentration of the anti-microbial agent in the microbe, and/or inhibit a host drug transporter. Also, for example, the amount of an opioid inhibitor of the ABC transporter may be an amount that facilitates the distribution of anti-microbial agents into tissues and/or cells of a subject where, in the absence of the inhibitor, the uninhibited ABC transporter facilitated efflux is so high as to prevent attainment of therapeutic concentrations of anti-microbial agents in those tissues and/or cells. The amount of a drug transporter inhibitor additionally may be an amount effective to enhance the therapeutic potency of and/or attenuate the adverse side effects of the anti-microbial agent. The optimum amounts of a drug transporter inhibitor administered alone or in combination with a therapeutic agent will of course depend upon the particular drug transporter inhibitor and anti-microbial agent used, the carrier chosen, the route of administration, and/or the pharmacokinetic properties of the subject being treated.

The present invention is described in the following examples which are set forth to aid in the understanding of the invention, and should not be construed to limit in any way the invention as defined in the claims which follow thereafter.

### EXAMPLE 1

### Assays of Human PGP-Mediated Transport with Opioid Receptor Antagonists

Porcine kidney-derived, LLC-PKI (American Type Culture Collection, Manassa, Va., A.T.C.C., #CL-101), cells expressing human PGP cDNA (designated 15B-J) were cultured in 24 well Falcon™ culture inserts at 37° C. on an orbital shaker. Transport assays were conducted in 24 well Falcon™ culture inserts with Hanks Balanced Salt Solution (HBSS) buffered with the addition of 10 mM HEPES (pH 7.2).

The test substances, naloxone, naltrexone and nalmefene, were purchased from Sigma-Aldrich (St. Louis, Mo.). Stock solutions of the compounds were made in DMSO, and dilutions of these in transport buffer were prepared for assay in the monolayers. The DMSO concentration (0.55%) was constant for all conditions within the experiment. All test substance and control drug solutions prepared in HBSS/HEPES buffer contained 0.55% DMSO.

The test substance was added to the donor and receiver chambers. Duplicate monolayers and thirteen nominal test substance concentrations of 0.0001, 0.0003, 0.001, 0.003, 0.01, 0.03, 0.1, 0.3, 1.0, 3.0, 10, 30 and 100 μM were used. PGP substrate [³H]-digoxin, at 5 μM was added to the donor chamber (either the apical or basolateral chamber depending on the direction of transport). After an incubation time of 90 minutes, a sample from the receiver chamber was analyzed for the amount of digoxin present. The positive control for inhibition was 25 μM ketoconazole added to donor and receiver chambers with 5 μM [³H]-digoxin added to the donor chamber. The negative control for inhibition was 5 μM [³H]-digoxin added to the donor chamber (either the apical or basolateral chamber depending on the direction of transport) with Hanks Balanced Salt Solution (HBSS) buffered with the addition of 10 mM HEPES (pH 7.2) and DMSO at 0.55% in the receiver chamber.

The rate of digoxin transported from the apical chamber to the basolateral chamber (A to B) and from the basolateral chamber to the apical chamber (B to A) was measured and apparent permeability Pₐₚₚ constants calculated. The polarization ratio P_{app B to A}/P_{app A} was calculated. A lower polarization ratio in the 15B-J cells with test substance relative to that without test substance provides evidence for inhibition of PGP-mediated digoxin transport by the test substance.

Transport of 5 μM [3H]-digoxin was measured following coincubation with the test substances at nominal concentrations in the range of 0 to 100 μM. Inhibition of digoxin transport was calculated by comparison of the digoxin polarization ratio in the presence of the test substance, to the ratio in the absence of test substance. The positive control for inhibition was 25 μM ketoconazole coincubated with digoxin. The inhibition of PGP-mediated transport in human PGP-expressing porcine kidney cell monolayers by naloxone is summarized in Table 1.

TABLE 1
Naloxone inhibition of PGP-mediated transport

| | | | % | |
|---|---|---|---|---|
| | | | Inhibition | Ketoconazole |
| Naloxone | | Polarization | Of | Normalized |
| Concentration (μM) | | Ratio | Digoxin | % Inhibition of |
| Nominal | Measured | B→ A/A→ B) | Transport | Digoxin Transport |
| 0 | — | 3.7 | — | — |
| 0.0001 | 0.000021 | 3.5 | 4.4 | 6.2 |
| 0.0003 | 0.000138 | 3.5 | 6.0 | 8.4 |
| 0.001 | 0.00085 | 3.4 | 7.3 | 10 |
| 0.03 | 0.0021 | 3.6 | 4.0 | 5.7 |
| 0.01 | 0.0083 | 3.8 | −3.2 | −4.5 |
| 0.03 | 0.021 | 3.5 | 4.1 | 5.7 |
| 0.1 | 0.074 | 3.8 | −1.9 | −2.7 |
| 0.3 | 0.264 | 3.3 | 11.9 | 17 |
| 1.0 | 1.04 | 3.5 | 5.5 | 7.8 |
| 3.0 | 2.79 | 4.0 | −7.7 | −11 |
| 10 | 10.0 | 3.7 | 0.3 | 0.4 |
| 30 | 31.8 | 2.6 | 29 | 41 |
| 100 | 99.8 | 1.1 | 26 | 37 |

The inhibition of PGP-mediated transport in human PGP-expressing porcine kidney cell monolayers by naltrexone is summarized in Table 2.

TABLE 2
Naltrexone inhibition of PGP-mediated transport

| | | | Ketoconazole |
|---|---|---|---|
| | Polarization | % Inhibition | Normalized % |
| Naltrexone | ratio | of Digoxin | Inhibition of |
| Concentration (μM) | (B→ A/A→ B) | Transport | Digoxin Transport |
| 0 | 4.0 | — | — |
| 0.0001 | 3.6 | 10 | 13 |
| 0.0003 | 3.5 | 14 | 19 |
| 0.001 | 3.6 | 10 | 13 |
| 0.003 | 3.7 | 8 | 11 |
| 0.01 | 3.5 | 11 | 15 |
| 0.03 | 3.8 | 5 | 6.7 |
| 0.1 | 3.5 | 14 | 19 |
| 0.3 | 3.3 | 18 | 24 |
| 1.0 | 3.4 | 14 | 19 |
| 3.0 | 3.5 | 14 | 19 |
| 10 | 3.4 | 15 | 20 |
| 30 | 3.3 | 17 | 23 |
| 100 | 2.7 | 33 | 44 |

The inhibition of PGP-mediated transport in human PGP-expressing porcine kidney cell monolayers by nalmefene is summarized in Table 3.

TABLE 3
Nalmefene inhibition of PGP-mediated transport

| | | | Ketoconazole |
|---|---|---|---|
| | | | Normalized % |
| | | % Inhibition of | Inhibition of |
| Concentration | Polarization Ratio | Digoxin | Digoxin |
| Nalmefene (μM) | (B→ A/A→ B) | Transport | Transport |
| 0 | 4.5 | — | — |
| 0.0001 | 4.3 | 5.2 | 7.5 |
| 0.0003 | 4.2 | 7.2 | 10 |
| 0.001 | 4.4 | 2.8 | 4.0 |
| 0.003 | 4.3 | 5.1 | 7.4 |
| 0.01 | 4.3 | 3.9 | 7.4 |
| 0.03 | 4.8 | −7.2 | −10 |
| 0.1 | 4.5 | −0.3 | — |
| 0.3 | 4.8 | −5.6 | −10 |
| 1.0 | 4.6 | −2.6 | −3.2 |
| 3.0 | 4.0 | 10 | 14 |
| 10 | 3.9 | 13 | 19 |
| 30 | 2.9 | 35 | 51 |
| 100 | 2.6 | 42 | 61 |

Nalmefene, naltrexone and naloxone exhibited inhibitory behavior from about 0.1 μM to about 100 μM concentrations. Digoxin transport appears to have been inhibited at naloxone and naltrexone concentrations below 30 μM, however, the inhibition was not concentration-dependent. Digoxin transport was increasingly inhibited in response to increasing concentration of nalmefene at concentrations between 3 and 100 μM. The positive control, 25 μM ketoconazole, inhibited digoxin transport within the accepted range, indicating that the cell model performed as expected.

### EXAMPLE 2

### Assays of Human PGP-Mediated Transport with 6-β-Naltrexol

Porcine kidney-derived, LLC-PK₁, cells expressing human PGP cDNA (designated 15B-J) were cultured in 24 well Falcon™ culture inserts at 37° C. on an orbital shaker. Transport assays were conducted in 24 well Falcon™ culture inserts with Hanks Balanced Salt Solution (HBSS) buffered with the addition of 10 mM HEPES (pH 7.2).

The test substance, 6-β-naltrexol, was provided by LC Resources, Inc., (Walnut Creek, Calif.). Stock solutions of the compounds were made in DMSO, and dilutions of these in transport buffer were prepared for assay in the monolayers. The DMSO concentration (0.55%) was constant for all conditions within the experiment. All test substance and control drug solutions prepared in HBSS/HEPES buffer contained 0.55% DMSO.

The test substance was added to the donor and receiver chambers. Duplicate monolayers and thirteen nominal test substance concentrations of 0.0001, 0.0003, 0.001, 0.003, 0.01, 0.03, 0.1, 0.3, 1, 3, 10, 30 and 100 μM, were used. PGP substrate [³H]-digoxin, at 5 μM was added to the donor chamber (either the apical or basolateral chamber depending on the direction of transport). After an incubation time of 90 minutes, a sample from the receiver chamber was analyzed for the amount of digoxin present. The positive control for inhibition was 25 μM ketoconazole added to donor and receiver chambers with 5 μM [³H]-digoxin added to the donor chamber. The negative control for inhibition was 5 μM [³H]-digoxin added to the donor chamber (either the apical or basolateral chamber depending on the direction of transport) and Hanks Balanced Salt Solution (HBSS) buffered with the addition of 10 mM HEPES (pH 7.2) and DMSO at 0.55% in the receiver chamber.

Transport of 5 μM [³H]-digoxin was measured following coincubation with test substance 6-β-naltrexol, at nominal concentrations in the range of 0 to 100 μM. Inhibition of digoxin transport was calculated by comparison of the digoxin polarization ratio in the presence of the test substance, to the ratio in the absence of test substance. The positive control for inhibition was 25 μM ketoconazole coincubated with digoxin.

Digoxin efflux in the human PGP-expressing cell monolayers was slightly inhibited (mean of 8.5 +/−7.1%) by 6-β-naltrexol in the concentration range of 0.0001 to 30 μM (Table 4 The inhibition did not appear to be concentration-dependent. At 100 μM 6-β-naltrexol, however, digoxin transport was more strongly inhibited (28%). The positive control, 25 μM ketoconazole, inhibited digoxin transport within the accepted range, indicating that the cell model performed as expected.

TABLE 4
6-β-naltrexol inhibition of PGP-mediated transport

| | | % | Ketoconazole |
|---|---|---|---|
| Nominal | Polarization | Inhibition of | Normalized % |
| concentration | Ratio | Digoxin | Inhibition of Digoxin |
| Of 6-β-naltrexol | (B→ A/A→ B) | Transport | Transport |
| 0 | 4.7 | — | — |
| 0.0001 | 4.4 | 6.4 | 8.1 |
| 0.0003 | 4.7 | 0 | — |
| 0.001 | 4.8 | −2.1 | −2.6 |
| 0.003 | 4.7 | 0 | — |
| 0.01 | 4.6 | 2.1 | 2.6 |
| 0.03 | 4.2 | 11 | 14 |
| 0.1 | 3.8 | 19 | 24 |
| 0.3 | 4.3 | 9 | 11 |
| 1.0 | 4.0 | 15 | 19 |
| 3.0 | 4.2 | 11 | 14 |
| 10 | 4.0 | 15 | 19 |
| 30 | 4.0 | 15 | 19 |
| 100 | 3.4 | 28 | 35 |
| 25 μM | 1.0 | 79 | — |
| Ketoconazole | | | |

The test substance 6-β-naltrexol was an inhibitor of PGP-mediated digoxin transport, in the concentration range tested.

### EXAMPLE 3

### Assays of PGP-Associated ATPase Activity with Opioid Receptor Antagonists

The test substances, naloxone, naltrexone and nalmefene, were purchased from Sigma-Aldrich. Stock solutions of the compounds were made in DMSO, and dilutions of these in TRIS-MES buffer were prepared for PGP-ATPase assay in the human PGP membranes (BD Biosciences, Franklin Lakes, N.J., Gentest Cat. K228). The DMSO concentration (1.0%) was constant for all conditions within the experiment.

The test substances were incubated in the PGP membranes and supplemented with MgATP, with and without sodium orthovanadate present. Orthovanadate inhibits PGP by trapping MgADP in the nucleotide binding site. Thus, the ATPase activity measured in the presence of orthovanadate represents non-PGP ATPase activity and was subtracted from the activity generated without orthovanadate to yield vanadate-sensitive ATPase activity.

ATPase assays were conducted in 96-well microtiter plates. A 0.06 mL reaction mixture containing 40 μg PGP membranes, test substance, and 4 mM MgATP, in buffer containing 50 mM Tris-MES, 2 mM EGTA, 50 mM KCl, 2 mM dithiothreitol, and 5 mM sodium azide, plus organic solvent was incubated at 37° C. for 20 minutes. Triplicate incubations of ten test substance concentrations (of 0.003, 0.01, 0.03, 0.1, 0.3, 1.0, 3.0, 10, 30 and 100 μM) and the test vehicle without drug, were used. Identical reaction mixtures containing 100 μM sodium orthovanadate were assayed in parallel. The reactions were stopped by the addition of 30 μl of 10% SDS+Antifoam A. The incubations were followed with addition of 200 μl of 35 mM Ammonium Molybdate in 15 mM Zinc Acetate:10% Ascorbic Acid (1:4) and incubated for an additional 20 minutes at 37° C. Additionally, 0.06 mL aliquots of potassium phosphate standards prepared in the buffer described above, were incubated in the plates containing the test and control substances, with SDS and detection reagent added. The liberation of inorganic phosphate was detected by its absorbance at 800 nm and quantitated by comparing the absorbance to a phosphate standard curve. The concentration dependence of the PGP was analyzed for evidence of saturation of PGP-ATPase activity, and apparent kinetic parameters were calculated by non-linear regression. The positive control for stimulation of ATPase activity was 20 μM verapamil. Ketoconazole at 25 μM (co-insulated with 20 μM verapamil) was tested as a possible inhibitor of verapamil-stimulated ATPase activity.

In a semi-quantitative assay for ATPase inhibition, nalmefene, naltrexone and naloxone were shown not to limit the ATPase activity associated with PGP1a as shown in Table 5.

TABLE 5
ATPase Activity

| | Activity (nmol/mg min) | | |
|---|---|---|---|
| Concentration (μM) | Naloxone | Naltrexone | Nalmefene |
| 100 | 1.8 | 4.6 | 3.3 |
| 30 | 1.9 | −2.0 | 2.3 |
| 10 | 2 | 2.0 | −0.5 |
| 3.0 | 1.7 | 2.1 | 0.2 |
| 1.0 | 0.4 | −0.8 | −1.6 |
| 0.3 | −0.6 | −1.4 | 1.9 |
| 0.1 | 3.6 | −0.7 | 1.1 |
| 0.03 | 0.7 | −1.6 | 1.2 |
| 0.01 | −0.8 | 0.0 | 1.3 |
| 0.003 | n.d. | −0.3 | 2.4 |
| 0.001 | −3.8 | n.d. | n.d. |
| 0.0 | 0.0 | 0.0 | 0.0 |
| 20 μM Verapamil + ketoconazole | 13.4* | 41.7** | n.d. |

*100 μM ketoconazole

### EXAMPLE 4

### Assays for Transport of Erythromycin by Human Pgp

Porcine kidney-derived, LLC-PK₁, cells expressing human PGP cDNA (designated 15B-J) and the untransferred control cell line were cultured in 24 well Falcon™ culture inserts at 37° C. on an orbital shaker. Transport assays were conducted in 24 well Falcon™ culture inserts with Hanks Balanced Salt Solution (HBSS) buffered with the addition of 10 mM HEPES (pH 7.2).

The test substance, erythromycin, was provided by Sigma-Aldrich (St. Louis, Mo.) A stock solution of the compound was made in DMSO, and diluted to 10 μM in transport buffer for assay in the cell monolayers.

Erythromycin at 10 μM was added to the donor chamber (either the apical or basolateral chamber depending on the direction of transport). A sample from the receiver chamber was analyzed for the amount of erythromycin present, after incubation times of 15, 30, 60, 90 and 120 minutes.

TABLE 6
PGP-mediated transport of erythromycin

| | | CLA | | | |
|---|---|---|---|---|---|
| | | (control) Monolayer | | 15B-J (PGP) Monolayer | |
| | | | Polarization | | Polarization |
| Minutes | | | Ratio | | Ratio |
| Incu- | Transport | pmoles in | (B→ A/ | pmoles in | (B→ A/ |
| bated | Direction | Receiver | A→ B) | Receiver | A→ B) |
| 15 | A→ B | 0.77 | 0.4 | 3.2 | 2.2 |
| | B→ A | 0.33 | | 7.0 | |
| 30 | A→ B | 1.47 | 0.7 | 4.5 | 2.8 |
| | B→ A | 0.84 | | 10 | |
| 60 | A→ B | 1.9 | 1.1 | 7.0 | 4.0 |
| | B→ A | 1.7 | | 22 | |
| 90 | A→ B | 3.3 | 1.1 | 8.8 | 5.5 |
| | B→ A | 2.5 | | 35 | |
| 120 | A→ B | 6.3 | 1.0 | 17.9 | 4.0 |
| | B→ A | 4.5 | | 43 | |

Erythromycin was significantly transported by human PGP.

### EXAMPLE 5

### Assays of PGP-Associated ATPase Activity with Erythromycin

The effect of erythromycin on PGP-associated ATPase activity was also assayed. Erythromycin was incubated in human PGP membranes (BD Biosciences, Franklin Lakes, N.J., Gentest Cat. K228) and supplemented with MgATP, with and without sodium orthovanadate present. ATPase assays were conducted in 96-well microtiter plates. A 0.06 mL reaction mixture containing 40 μg PGP membranes, erythromycin, and 4 mM MgATP, in buffer containing 50 mM Tris-MES, 2 mM EGTA, 50 mM KCl, 2 mM dithiothreitol, and 5 mM sodium azide, plus organic solvent (1% DMSO) was incubated at 37° C. for 20 minutes. Triplicate incubations of erythromycin at concentrations of 0.1 μM, 0.2 μM, 0.4 μM, 0.8 μM, 1.6 μM, 3.13 μM, 6.25 μM, 12.5 μM, 25 μM, 50 μM and 100 μM as shown below, and the test vehicle without drug, were used. Identical reaction mixtures containing 100 μM sodium orthovanadate were assayed in parallel. The reactions were stopped by the addition of 30 μl of 10% SDS+Antifoam A. The incubations were followed with addition of 200 μl of 35 mM Ammonium Molybdate in 15 mM Zinc Acetate:10% Ascorbic Acid (1:4) and incubated for an additional 20 minutes at 37° C. Additionally, 0.06 mL aliquots of potassium phosphate standards prepared in the buffer described above, were incubated in the plates containing the test and control substances, with SDS and detection reagent added. The liberation of inorganic phosphate was detected by its absorbance at 800 nm and quantitated by comparing the absorbance to a phosphate standard curve. The concentration dependence of the PGP was analyzed for evidence of saturation of PGP-associated ATPase activity, and apparent kinetic parameters were calculated by non-linear regression. The positive control for stimulation of ATPase activity was 20 μM verapamil.

In a semi-quantitative assay for ATPase inhibition, erythromycin was shown to stimulate PGP-associated ATPase activity (see Table 7). This is consistent with erythromycin being effectively transported by human PGP.

TABLE 7
ATPase Activity

| | ATPase |
|---|---|
| Erythromycin | Activity |
| Concentration (μM) | (nmol/mg min) |
| 100 | 15.6 |
| 50 | 14.0 |
| 25 | 8.2 |
| 12.5 | 2.8 |
| 6.25 | 0.6 |
| 3.13 | 0.5 |
| 1.6 | 2.0 |
| 0.8 | 0.1 |
| 0.4 | 0.8 |
| 0.2 | 2.0 |
| 0.1 | 1.1 |
| 0.0 | 0.0 |
| 20 μM Verapamil | 31 |

### EXAMPLE 6

### Assays for Transport of Tobramycin by Human PGP

Porcine kidney-derived, LLC-PK₁, cells expressing human PGP cDNA (designated 15B-J) and the untransferred control cell line are cultured in 24 well Falcon™ culture inserts at 37° C. on an orbital shaker. Transport assays are conducted in 24 well Falcon™ culture inserts with Hanks Balanced Salt Solution (HBSS) buffered with the addition of 10 mM HEPES (pH 7.2).

The test substance, tobramycin, is provided by Sigma-Aldrich (St. Louis, Mo.). A stock solution of the compound is made in DMSO, and diluted to 10 μM in transport buffer for assay in the cell monolayers.

Tobramycin, at 10 μM was added to the donor chamber (either the apical or basolateral chamber depending on the direction of transport). A sample from the receiver chamber was analyzed for the amount of tobramycin present, after incubation times of 30, 60, 90 and 120 minutes.

### EXAMPLE 7

### Tobramycin Stimulates PGP-Associated Atpase Activity

The effect of tobramycin on PGP-associated ATPase activity was also assayed. The tobramycin was incubated in human PGP membranes (BD Biosciences, Franklin Lakes, N.J., Gentest Cat. K228) and supplemented with MGATP, with and without sodium orthovanadate present. ATPase assays were conducted in 96-well microtiter plates. A 0.06 mL reaction mixture containing 40 μg PGP membranes, tobramycin, and 4 mM MgATP, in buffer containing 50 mM Tris-MES, 2 mM EGTA, 50 mM KCl, 2 mM dithiothreitol, and 5 mM sodium azide, plus organic solvent (1% DMSO) was incubated at 37° C. for 20 minutes. Triplicate incubations of tobramycin concentrations of 0.1 μM, 0.2 μM, 0.4 μM, 0.8 μM, 1.6 μM, 3.13 μM, 6.25 μM, 12.5 μM, 25 μM, 50 μM and 100 μM and the test vehicle without drug, were used. Identical reaction mixtures containing 100 μM sodium orthovanadate were assayed in parallel. The reactions were stopped by the addition of 30 μl of 10% SDS+Antifoam A. The incubations were followed with addition of 200 μl of 35 mM Ammonium Molybdate in 15 mM Zinc Acetate:10% Ascorbic Acid (1:4) and incubated for an additional 20 minutes at 37° C. Additionally, 0.06 mL aliquots of potassium phosphate standards prepared in the buffer described above, were incubated in the plates containing the test and control substances, with SDS and detection reagent added. The liberation of inorganic phosphate was detected by its absorbance at 800 nm and quantitated by comparing the absorbance to a phosphate standard curve. The concentration dependence of the PGP was analyzed for evidence of saturation of PGP-ATPase activity, and apparent kinetic parameters were calculated by non-linear regression. The positive control for stimulation of ATPase activity was 20 μM verapamil.

In a semi-quantiative assay for ATPase inhibition, tobramycin was shown to stimulate PGP-associated ATPase activity (see Table 8). The assay may be further optimized by enhancing the solubility of tobramycin.

TABLE 8
ATPase Activity

| | ATPase |
|---|---|
| Tobramycin | Activity |
| Concentration (μM) | (nmol/mg min) |
| 100 | 1.5 |
| 50 | 0.3 |
| 25 | 0.2 |
| 12.5 | −1.0 |
| 6.25 | −1.4 |
| 3.13 | −2.6 |
| 1.6 | −2.8 |
| 0.8 | −3.3 |
| 0.4 | −2.6 |
| 0.2 | −2.6 |
| 0.1 | −1.6 |
| 0.0 | 0.0 |
| 20 μM Verapamil | 27 |

### EXAMPLE 8

### Molecular Modeling Analysis

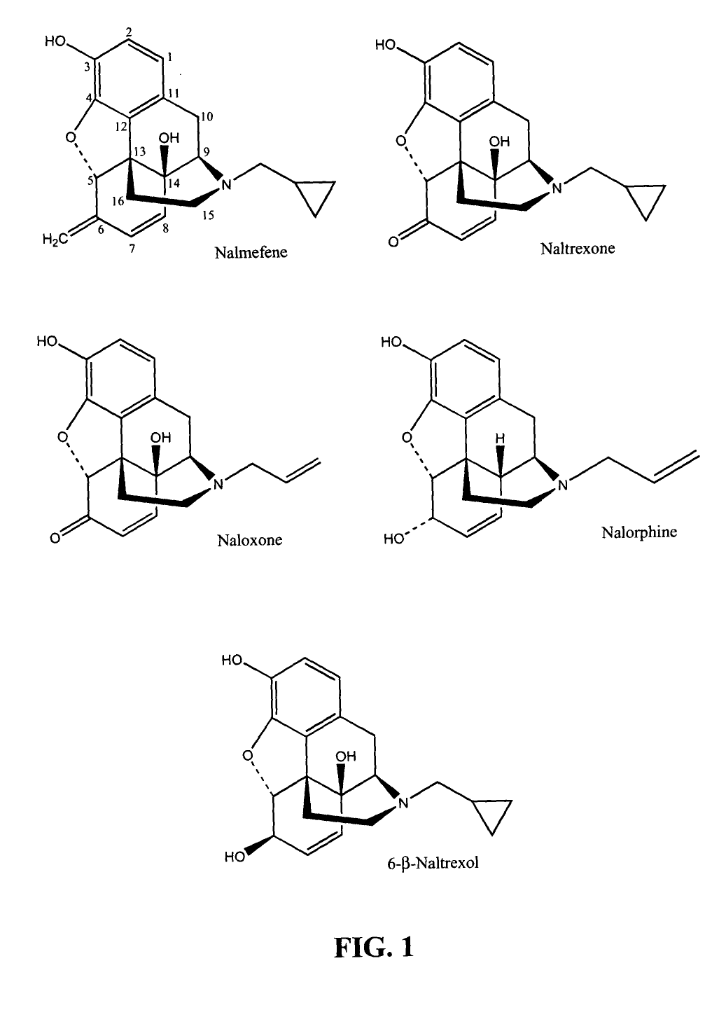

A molecular modeling analysis was performed on a series of compounds, including opioid analogues, to elucidate their mode of interaction and to determine, a pharmacophore for drug transporter inhibitors useful in the present invention. Exemplary compounds in this study were nalmefene, naltrexone, naloxone, 6-β-naltrexol and nalorphine. The structures of compounds are illustrated in FIG. 1. The compounds are structurally very similar, and exhibit two measured activities. “Activity 1” is characterized by a low capacity, high affinity binding site with activity ranging from 0.3 nM to greater than 200 μM. On the other hand, “activity 2” is characterized by a high capacity, low affinity binding site with activity ranging from 10 μM to greater than 100 μM. Table 9 provides the biological activities for each of the exemplary compounds.

TABLE 9
Biological Activity of Exemplary Compounds

| Compound | Activity 1 | Activity 2 |
|---|---|---|
| Nalmefene | 0.3 nM | 100 μM |
| Naltrexone | 0.3 nM | 100 μM |
| Naloxone | 1.0 nM | 30 μM |
| 6-β-Naltrexol | 0.1 nM | 100 μM |
| Nalorphine | N/A | N/A |

In performing the calculations for the molecular modeling analysis, two assumptions were made. First, nalorphine exhibits no measurable activity. Second, the structures of the compounds as represented in the Merck Index represent is the active form of the compound.

An important difference in these compounds is that nalorphine lacks the hydroxyl group in the central ring at position 14 (see, e.g., FIG. 1), indicating that this hydroxyl group is a requirement for activity. The most active compounds (nalmefene and naltrexone) each have a hydrophobic group (cyclopropyl) tethered to the nitrogen, indicating that a hydrophobic moiety is partially responsible for the higher activity in these compounds. This moiety may be viewed as a necessary, but not sufficient condition, since several of the inactive compounds also possess this hydrophobic region. Initial activity data suggest that the electron density present at this location in naloxone (due to the ethylene substituent [C═C]) is contributory to its lower activity. The observation that 6-β-Naltrexol is even less active is attributed to the hydroxyl substituent at the 6 position being oriented β to the ring system, perhaps penetrating a sterically limited region in the transporter.

In summary, the analysis indicates that the presence of the hydroxyl group at the 14-position may be required for activity, since nalorphine, with no calculated activity, lacks this moiety. In addition, the two most active compounds (nalmefene and naltrexone) possess an ethylene group and a carbonyl group respectively at the 6-position. This may represent a requirement for electron density at this position, rather than a hydrogen-bond acceptor site, as there is only a one order of magnitude difference in activity (0.3 nM vs. 3 nM) between the ethylene group (nalmefene) and the carbonyl group (naltrexone). There is a potential steric limit for substituent size or directionality at the 6-position, based on the analysis of 6-β-Naltrexol indicates that its hydroxyl group in a direction that penetrates into this region. Finally, a hydrophobic group is required as the N-substituent for highest activity, as naloxone, with a double bond rather than the cyclopropyl group exhibits lower activity.

When the novel analysis described above is now considered in conjunction with a recent scientific article investigated the ability of a variety of peptidomimetic thrombin inhibitors to inhibit intestinal transport [Kamm et al., “Transport of peptidomimetic thrombin inhibitors with a 3-amino-phenylalanine structure: permeability and efflux mechanism in monolayers of a human intestinal cell line (Caco-2).” Pharm. Res. 18:1110-8 (2001)], it is possible to utilize additional structural information from Kamm to develop a model of interaction with PGP. Kamm et al. proposed that basic and acidic residues of amidino-phenylalanine-derived thrombin inhibitors mediate affinity to intestinal efflux pumps, presumably PGP and MRP. Structural information from Kamm et al. useful in the novel QSAR analysis of the present invention is summarized below:

TABLE 10
R-groups of compounds Kamm et al.

| Structure | R1 | R2 | R3 | X | R4 |
|---|---|---|---|---|---|
| 1 | Me | H | H | C | 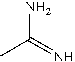 |
| 2 | H | COOH | H | C | 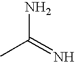 |
| 3 | H | COO-Me | H | C | 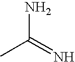 |
| 4 | H | H | COOH | C | 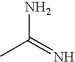 |
| 5 | H | H | COO-Me | C | 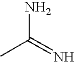 |
| 6 | COOH | H | H | C | 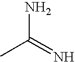 |
| 7 | COO-Me | H | H | C | 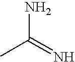 |
| 8 | COOH | H | H | C | 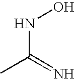 |
| 9 | COOH | H | H | C | 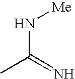 |
| 10 | H | H | H | N | 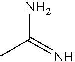 |
| 11 | 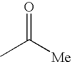 | H | H | N | 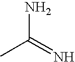 |
| (12) | Me | H | H | C | 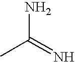 |
| 13 | Me | H | H | C | NH₂ |
| 14 | Me | H | H | C | —CH₂NH₂ |
| 15 | Me | H | H | C | 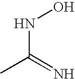 |
| 16 | Me | H | H | C | 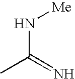 |

The intestinal permeability coefficients of the Kamm compounds were studied using Caco-2 monolayers and reverse-phase HPLC method for quantitation. Further the efflux ratios (transport from B to A:transport from A to B) were calculated. The efflux ratios for a selection of the Kamm compounds measured at 250 μM are provided in Table 11.

TABLE 11
Efflux Ratios at 250 μM

| | Efflux Ratio |
|---|---|
| Structure | B→A/A→B |
| 1 | 45.0 |
| 2 | 2.8 |
| 3 | 10.5 |
| 4 | 2.7 |
| 5 | 11.1 |
| 6 | 1.9 |
| 7 | 6.0 |
| 8 | 22.1 |
| 9 | 1.1 |
| 10 | 0.8 |
| 11 | 2.4 |

The efflux ratios the remaining Kamm compounds measured at 100 μM are provided in Table 12.

TABLE 12
Efflux Ratios at 100 μM

| | Efflux Ratio |
|---|---|
| Structure | B→A/A→B |
| 1 | 16.3 |
| 12 | 24.9 |
| 13 | 1.14 |
| 14 | 3.43 |
| 15 | 1.31 |
| 16 | 13.0 |

The comparable measurements for the opioid analogues are provided in Table 9. The data of Table 13 was obtained from the experiments described in Example 1. Efflux ratios normalized to 25 μM ketoconazole (Keto) are presented in parentheses after the measured ratios.

TABLE 13
Efflux Ratios of Opioid Analogues

| | | Hi Affinity/ | | | |
|---|---|---|---|---|---|
| | | Low Cap | | Low Affinity/Hi Cap | |
| | Keto | | B^{→} A/ | | B^{→} A/ |
| Structure | @25 μM | [C] μM | A^{→} B | [C] μM | A^{→} B |
| Nalmefene | 1.4 | 0.0003 | 4.2 (3.0) | 100 | 2.6 (1.9) |
| Naltrexone | 1.0 | 0.0003 | 3.5 (3.5) | 100 | 2.7 (2.7) |
| Naloxone | 1.1 | 0.001 | 3.4 (3.1) | 30 | 2.6 (2.4) |
| Naloxone | | | | 100 | 2.7 (2.5) |
| 6-β-Naltrexol | 1.0 | 0.0001 | 4.4 (4.4) | 100 | 3.4 (3.4) |

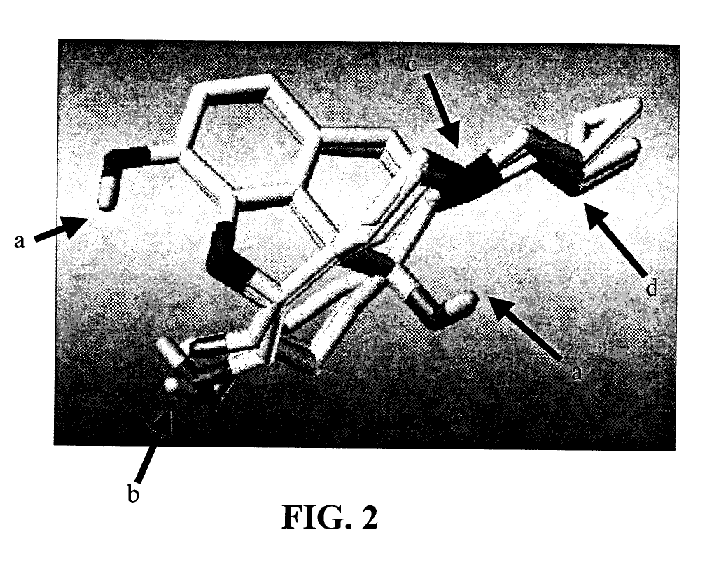

An overlay of the opioid analogue structures is presented in FIG. 2. All active (“Activity 1”) compounds share the following features: two hydroxyl groups (a) at positions 3 and 14, a furan ring system, a hydrophobic region in ring system, a region of electron density at position 6 (b), and a cyclic tertiary nitrogen (c) with an appended hydrophobic group (d).

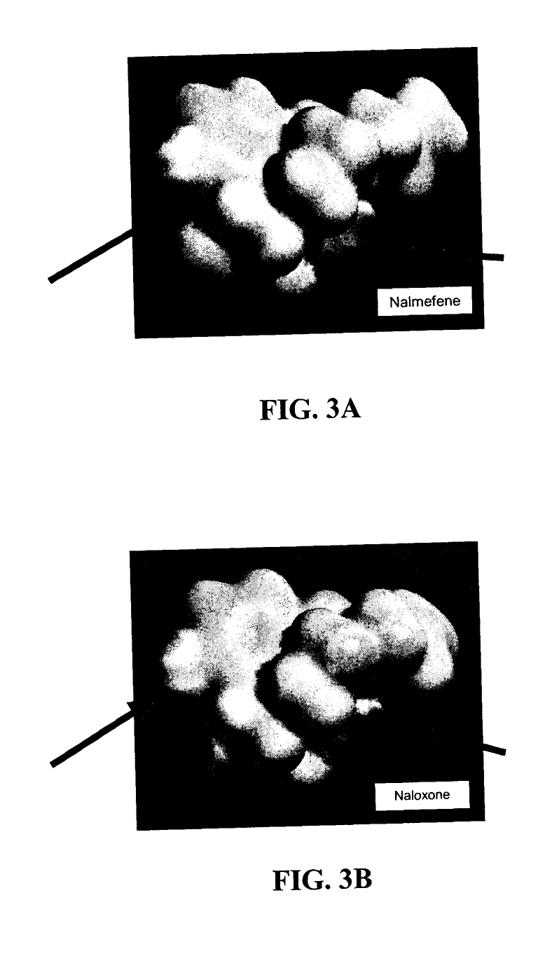

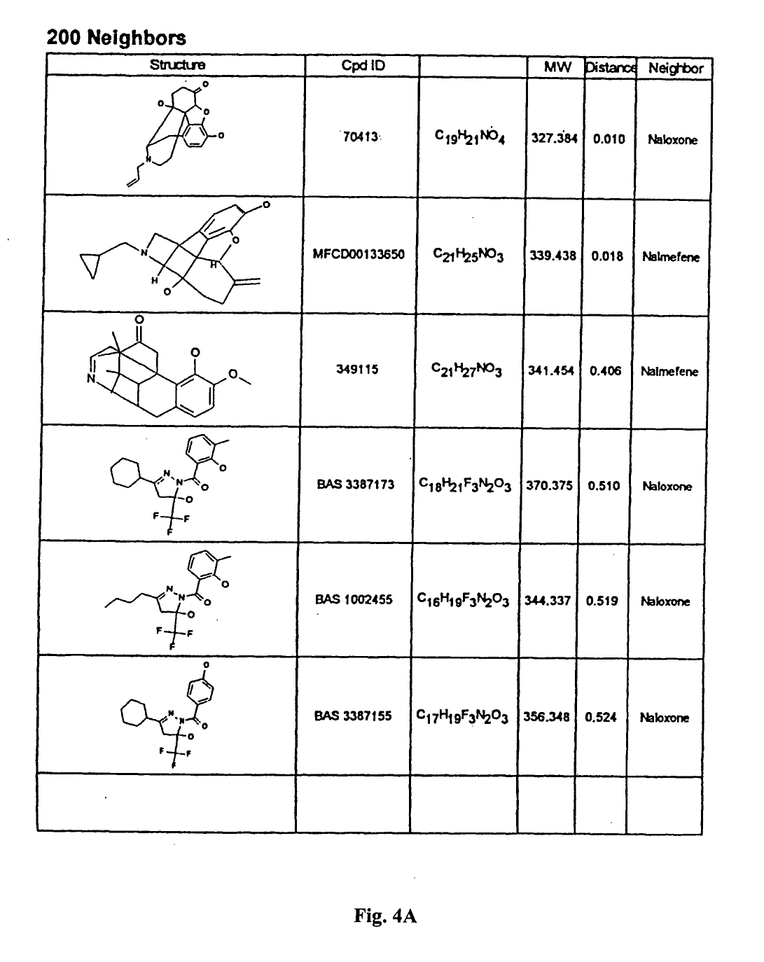

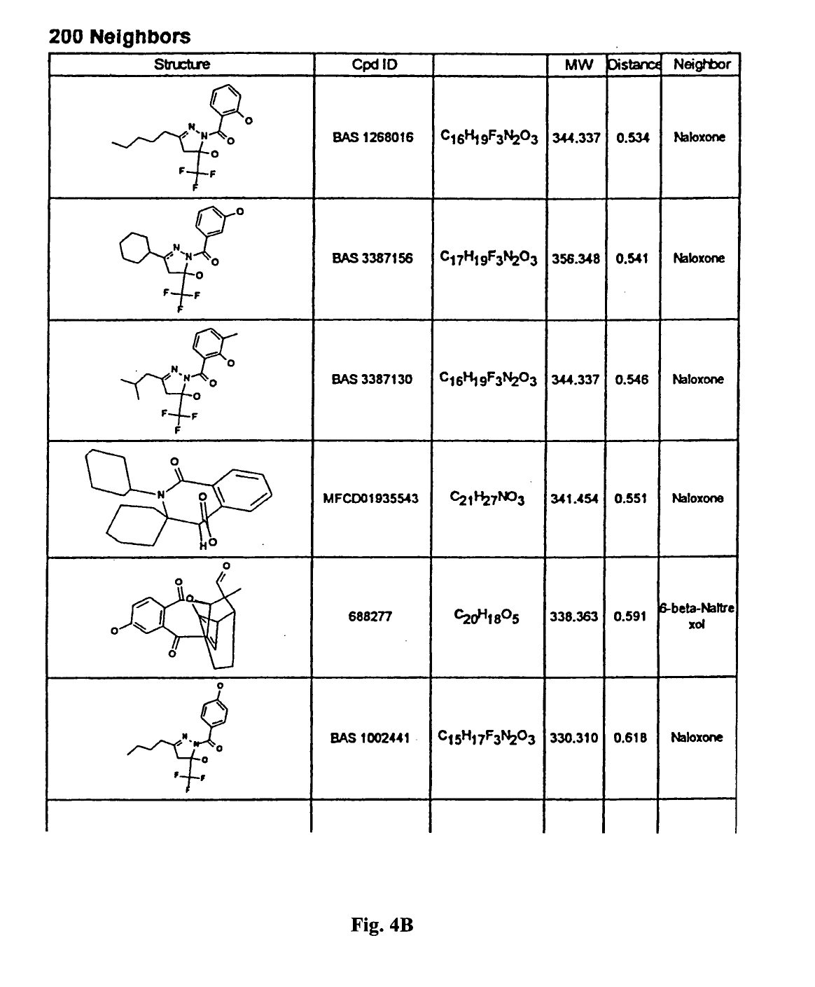

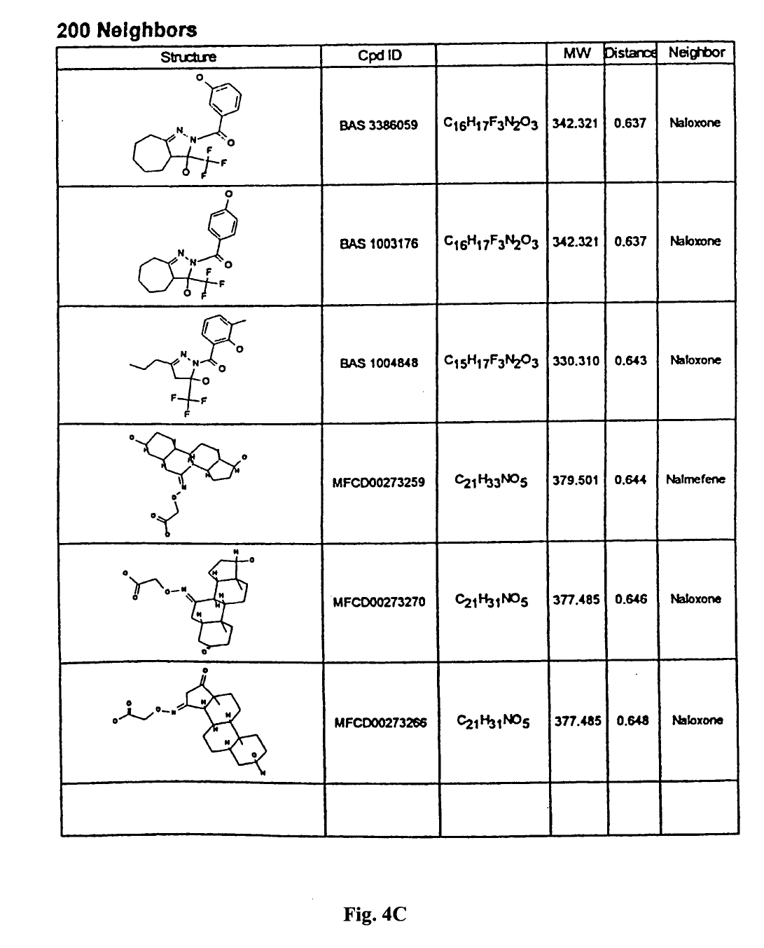

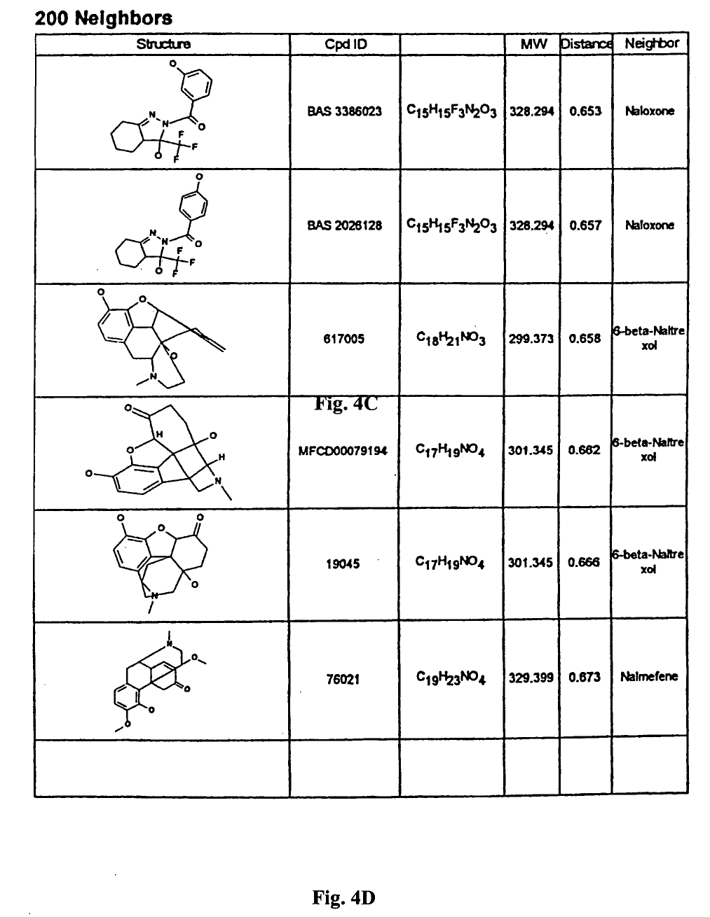

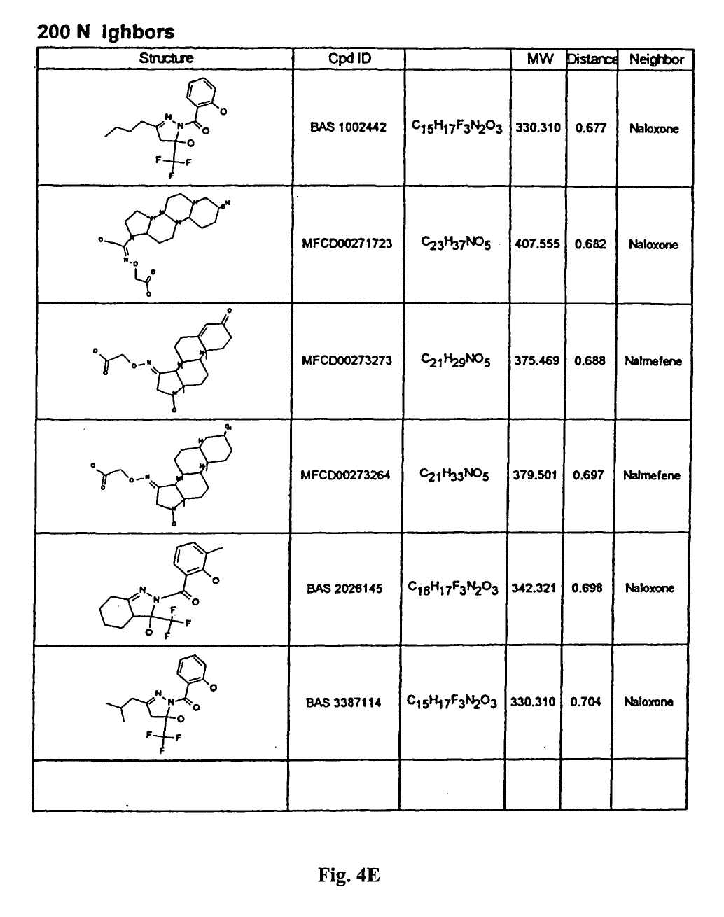

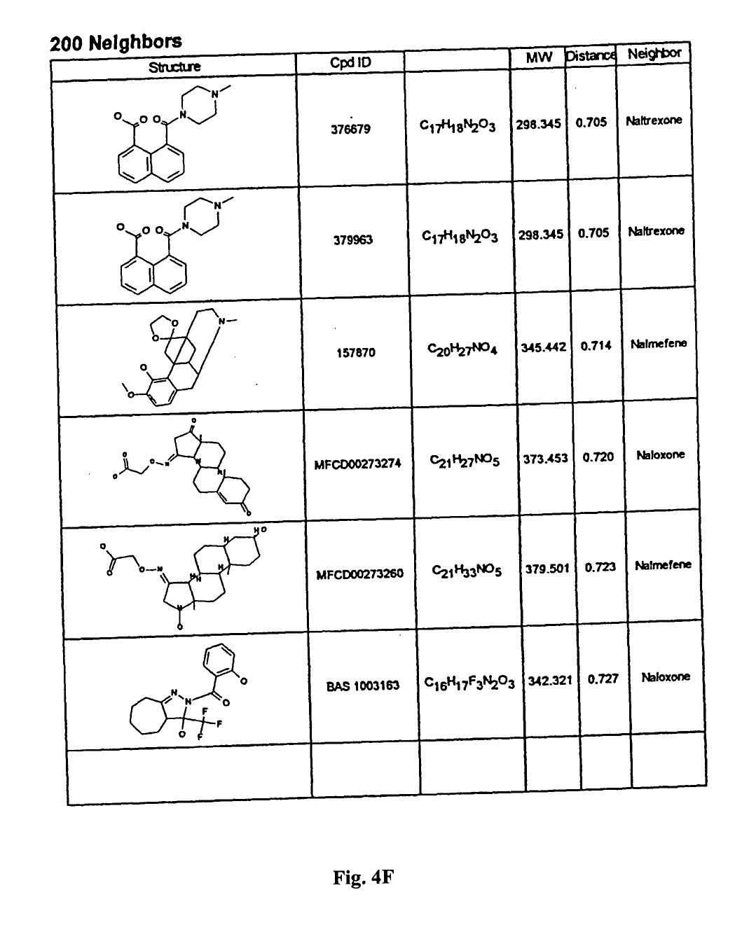

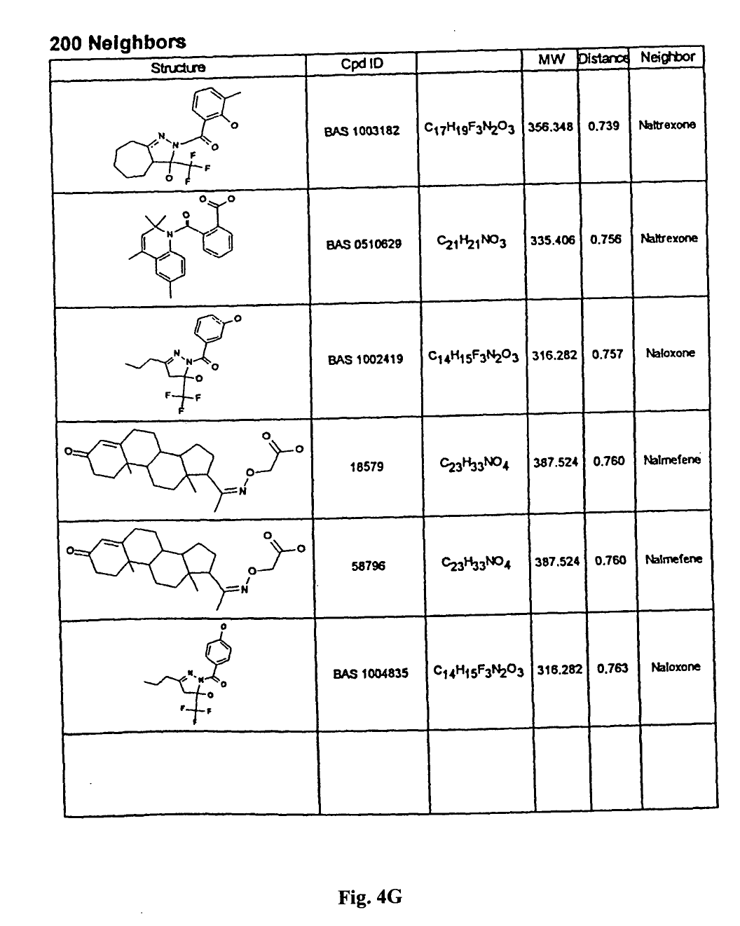

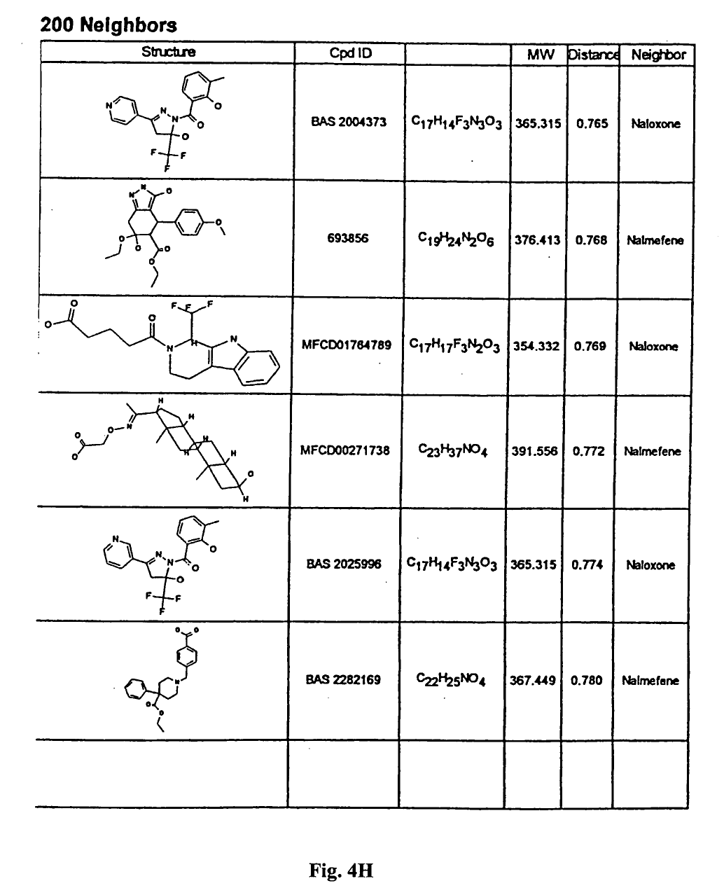

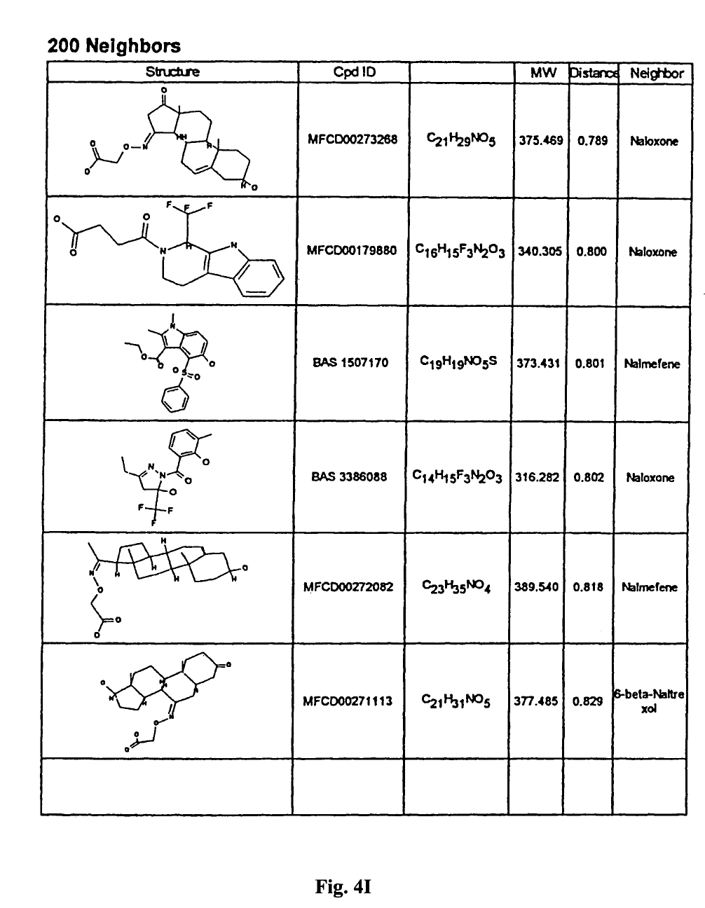

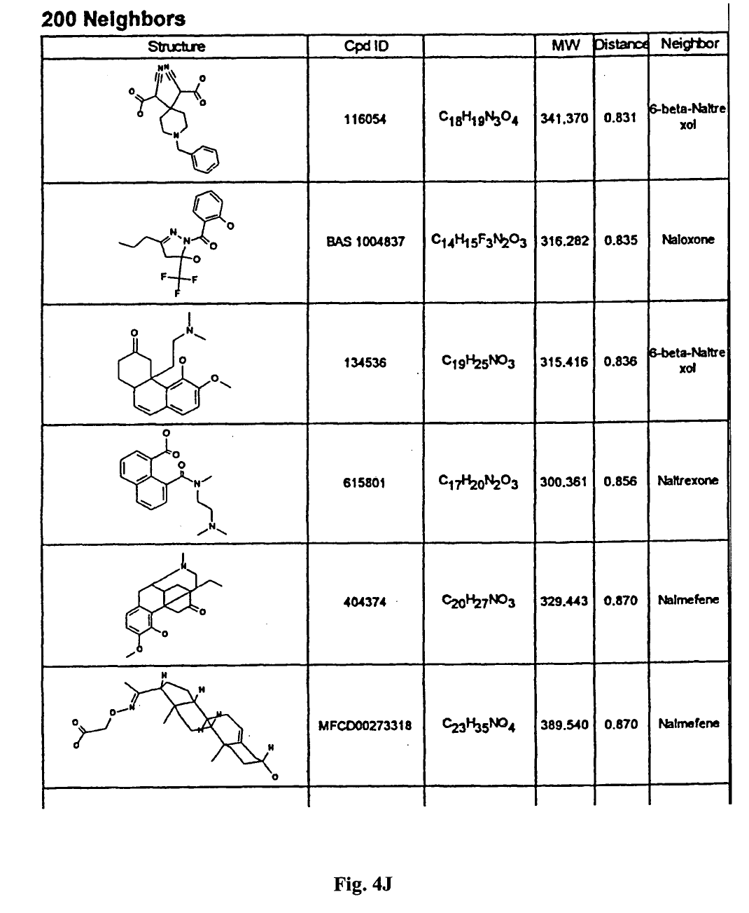

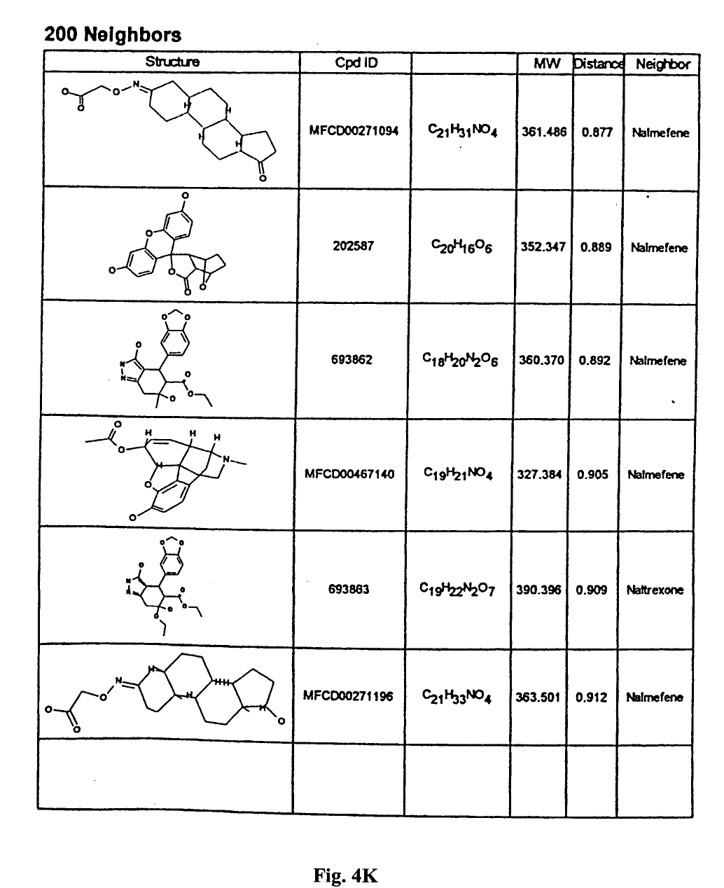

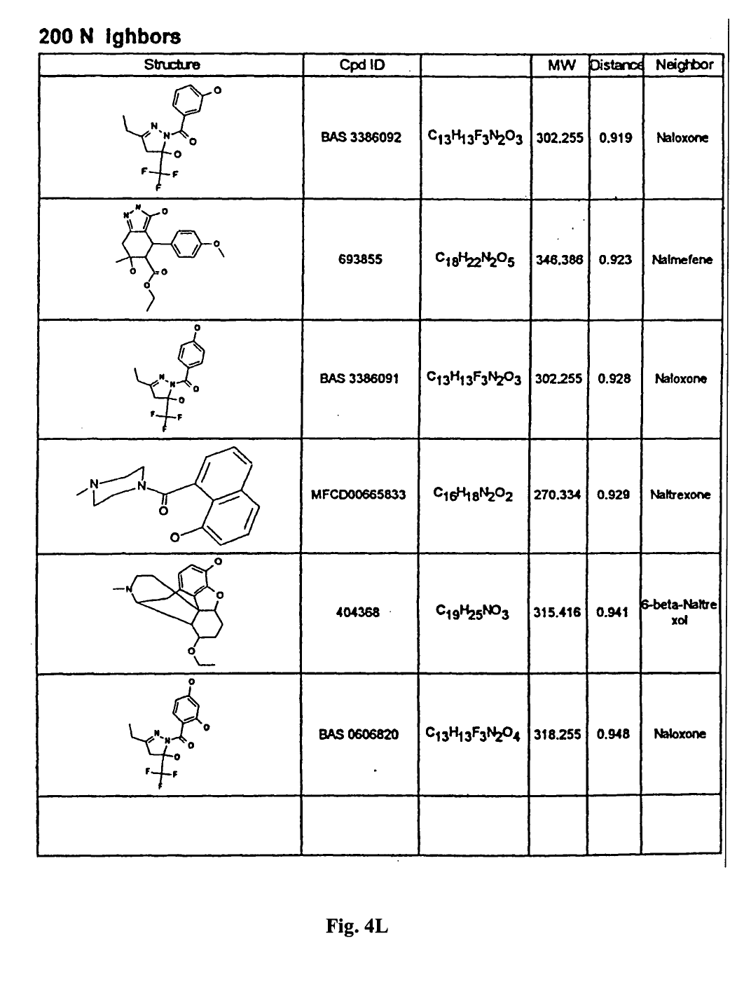

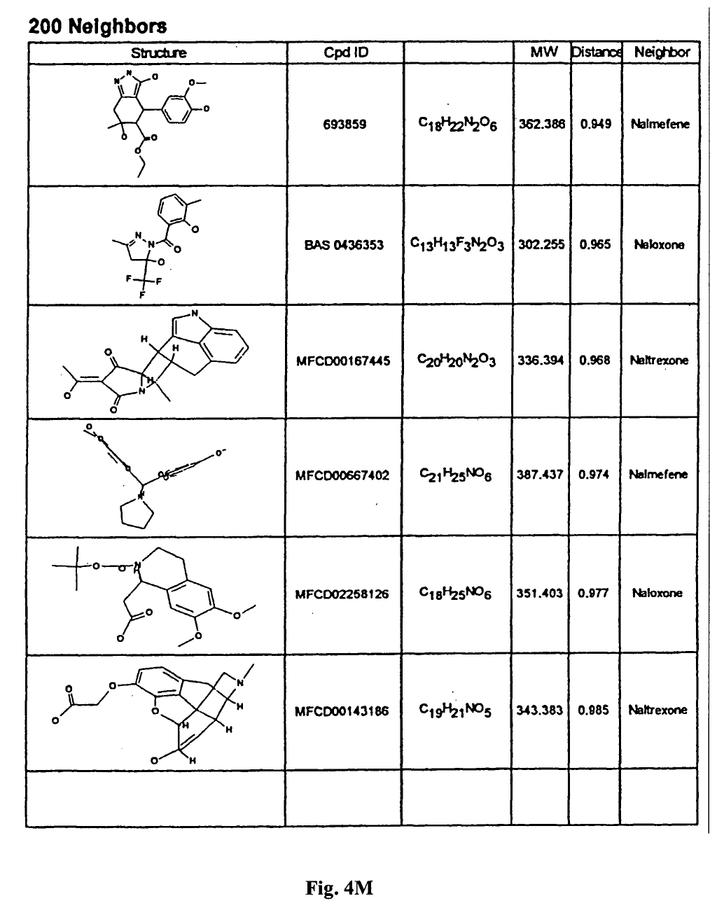

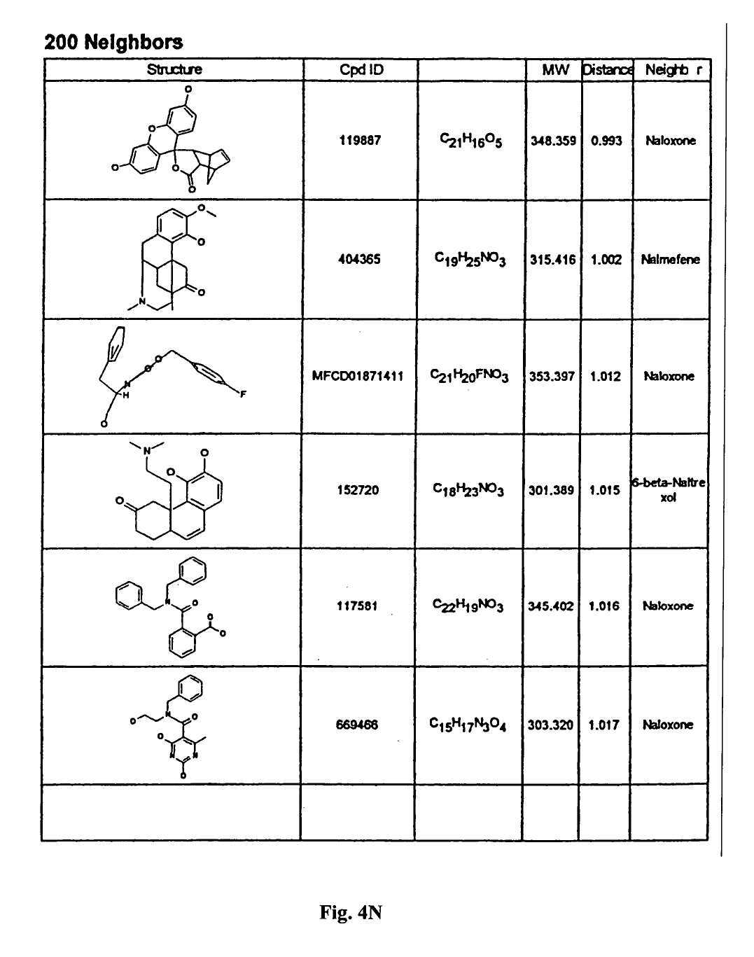

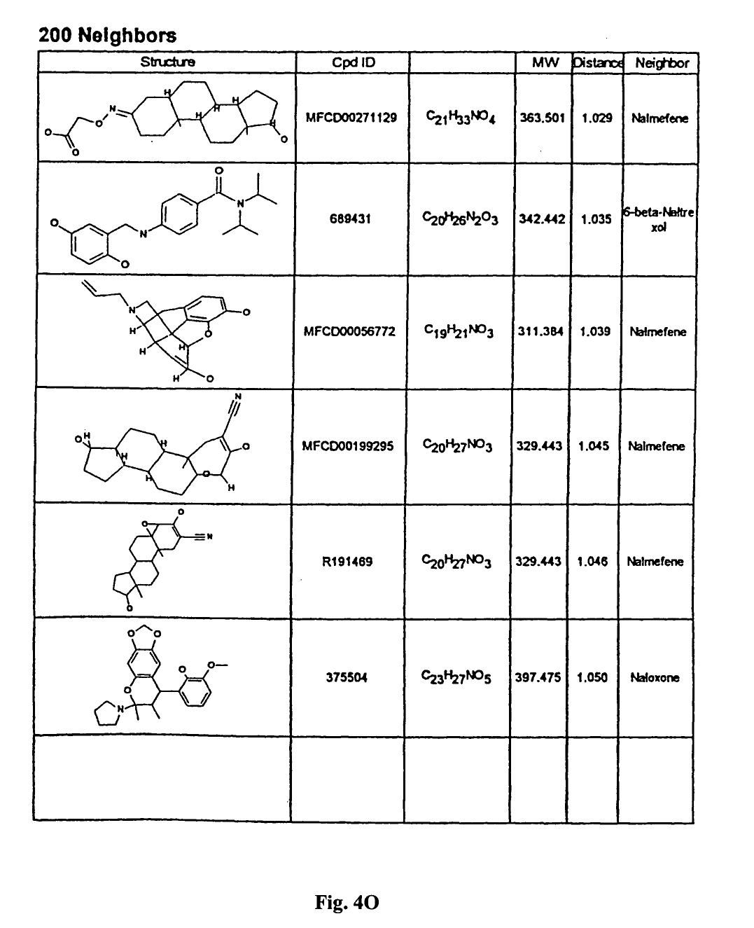

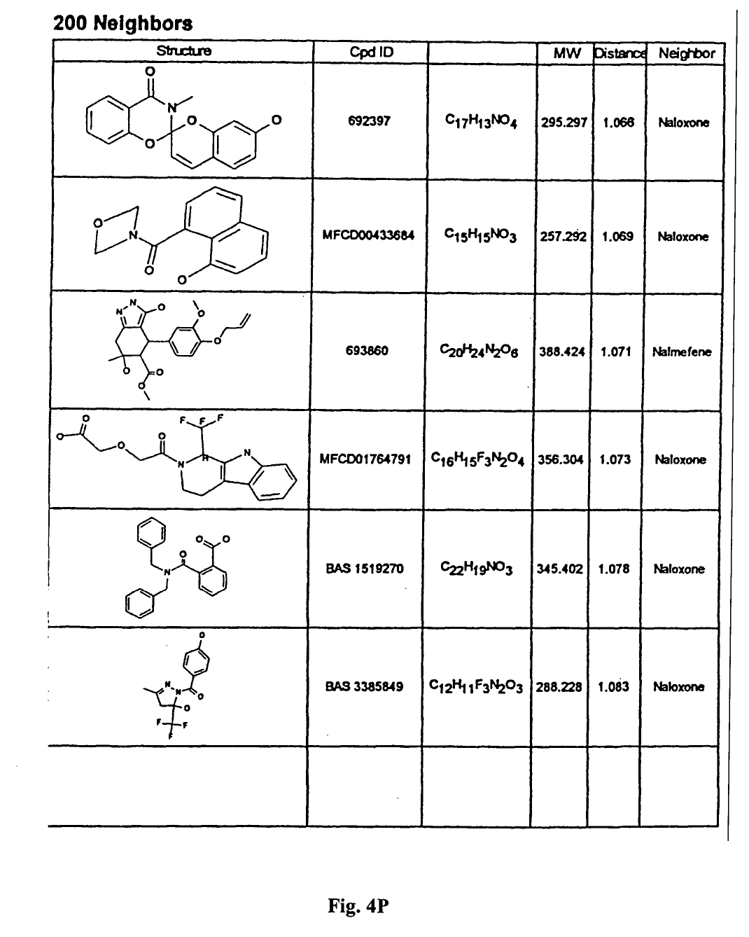

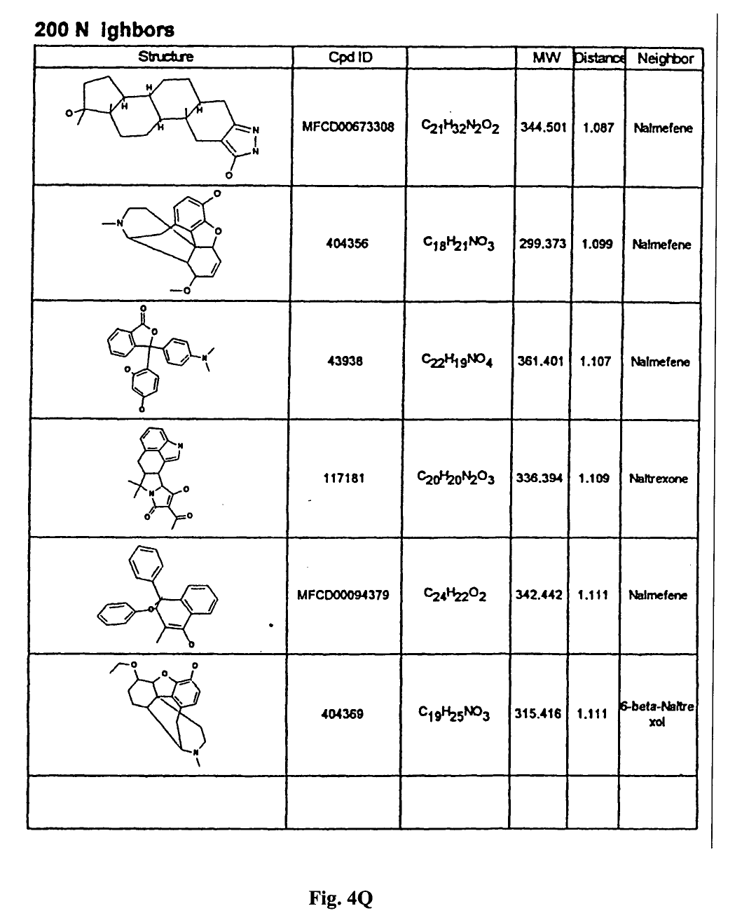

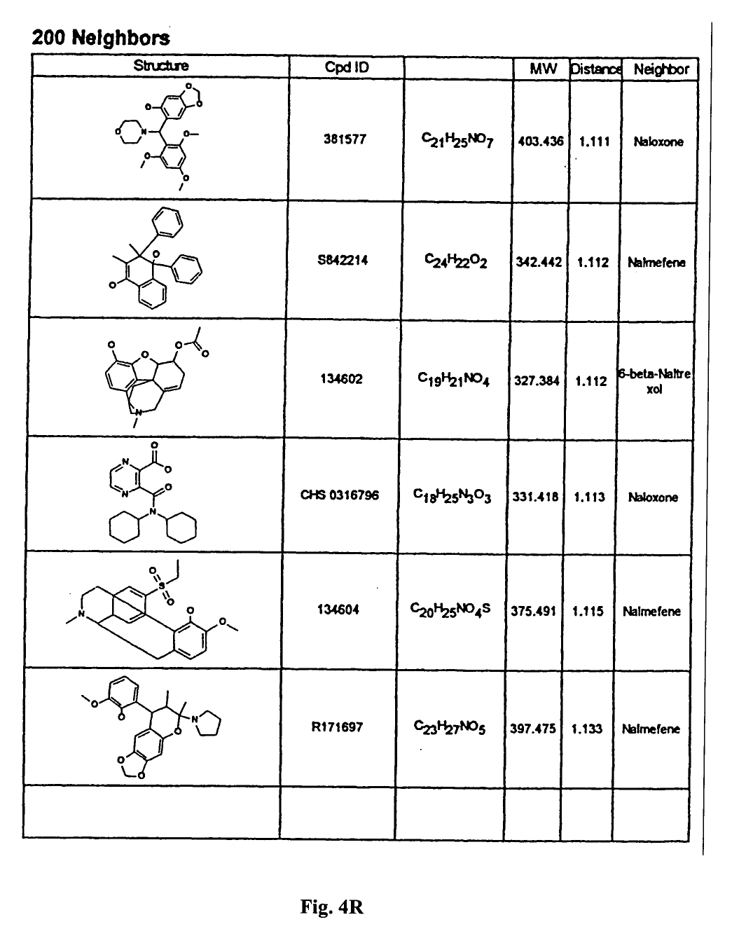

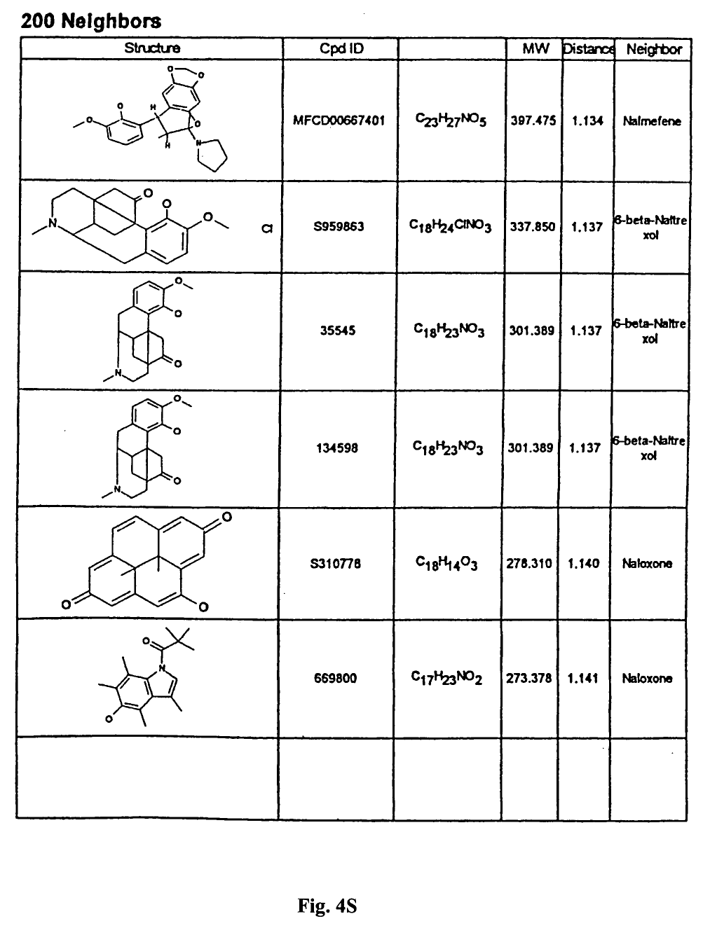

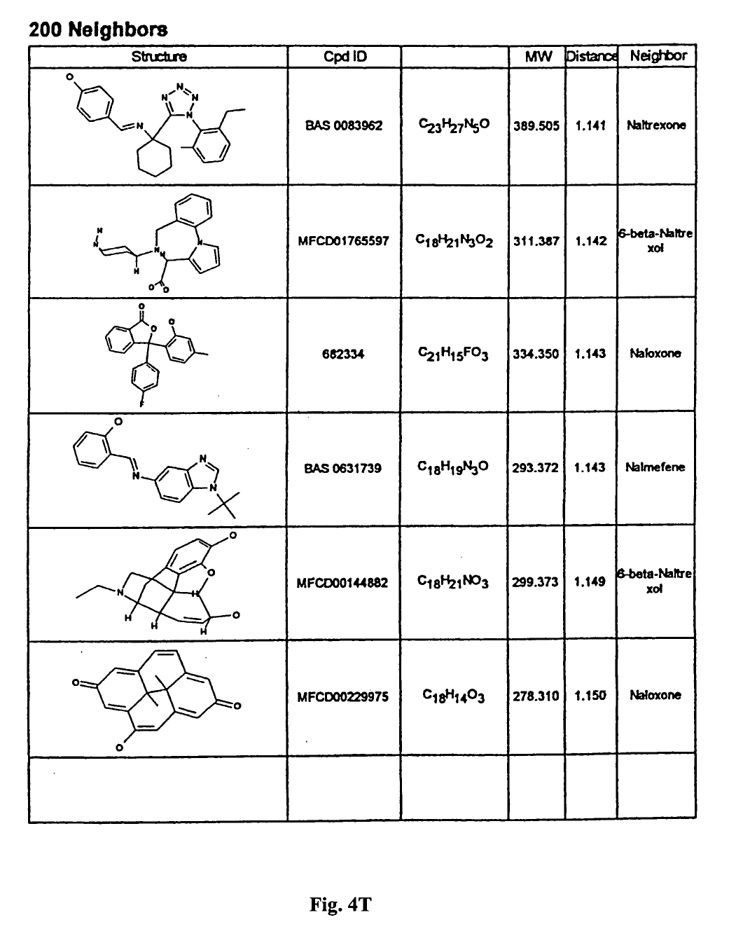

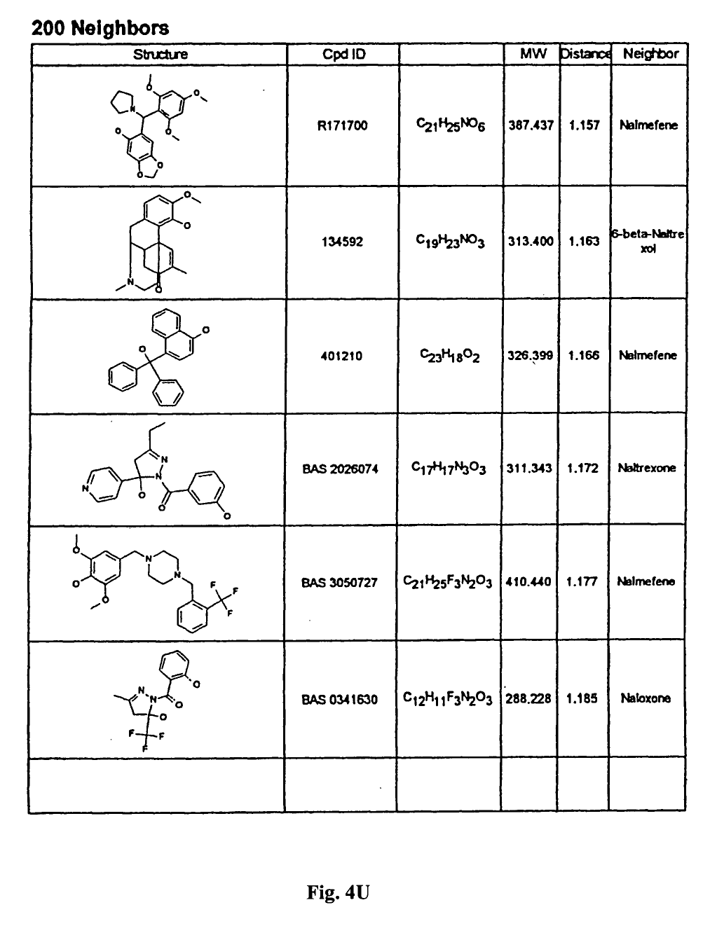

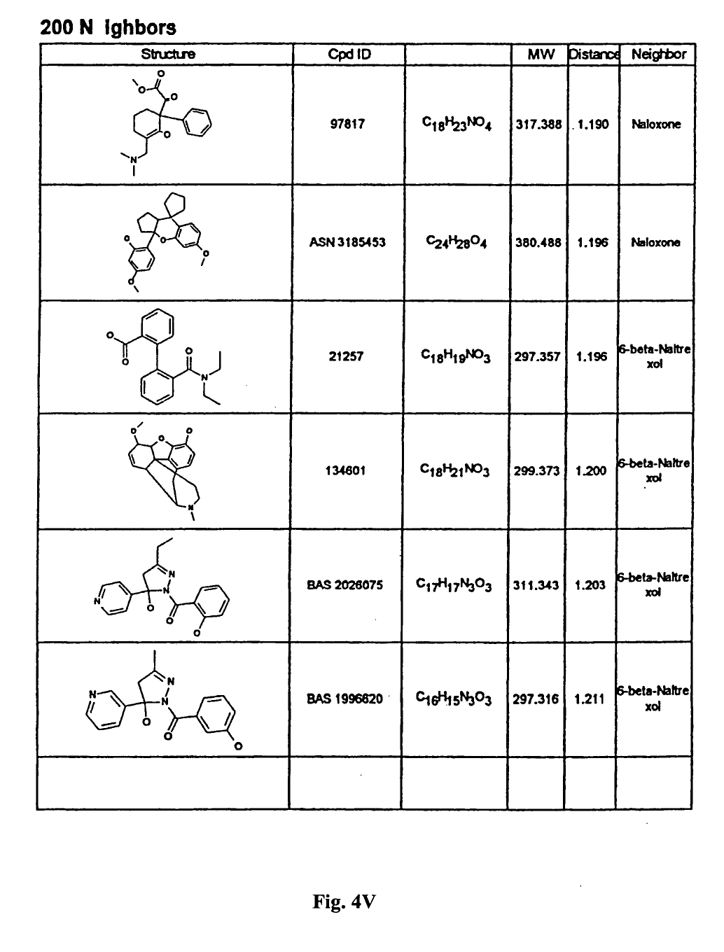

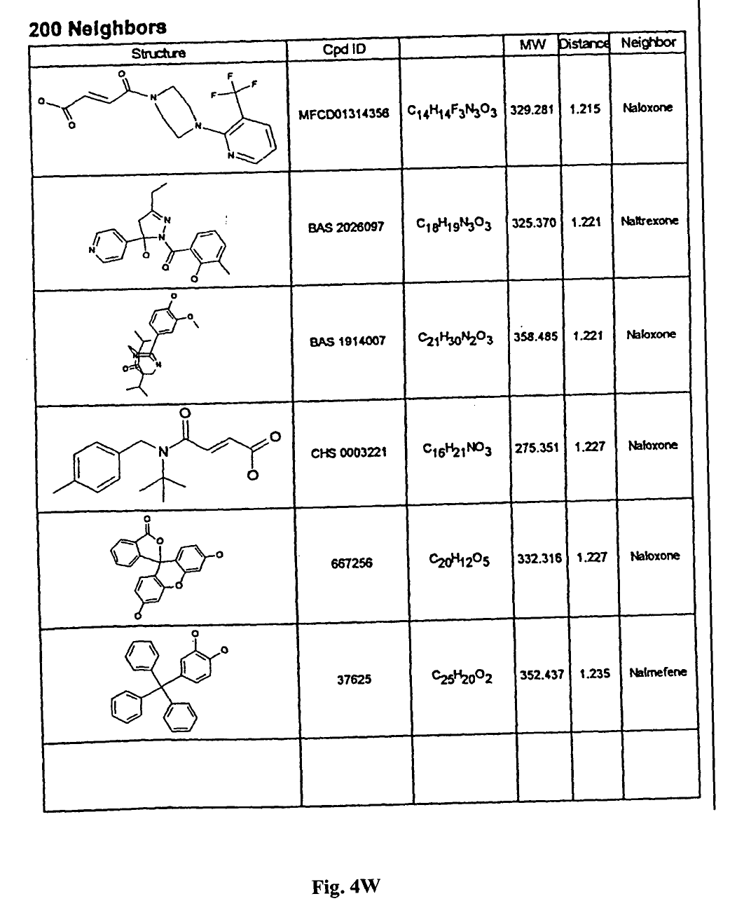

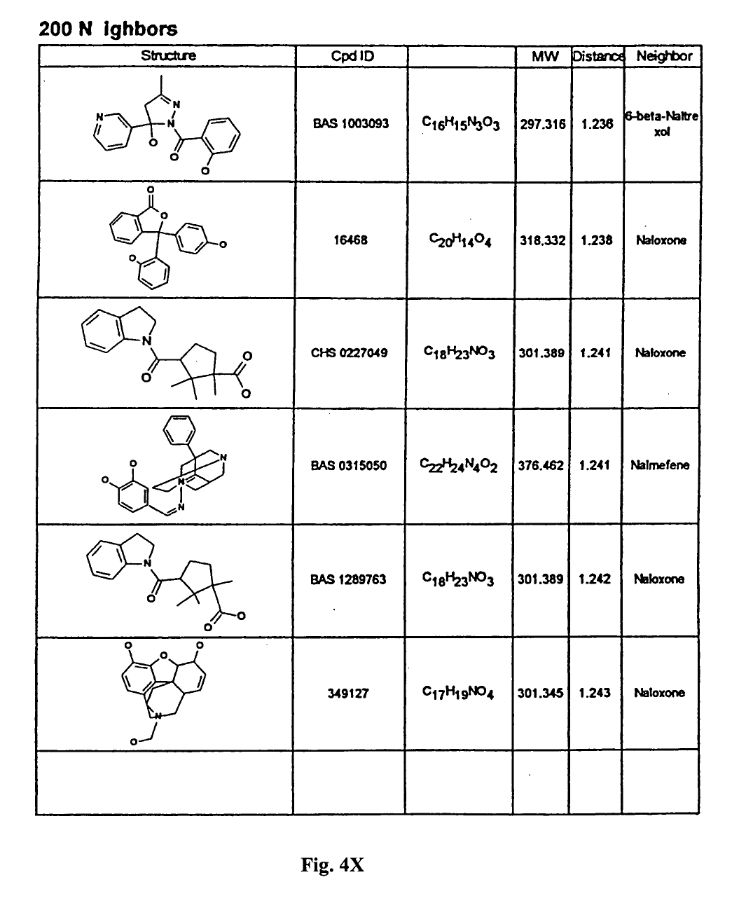

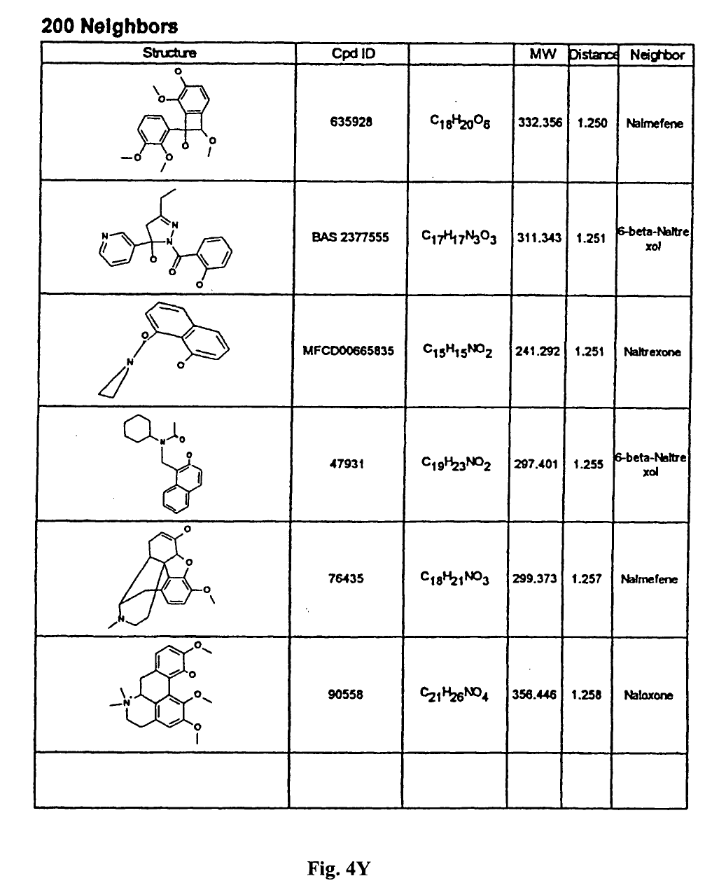

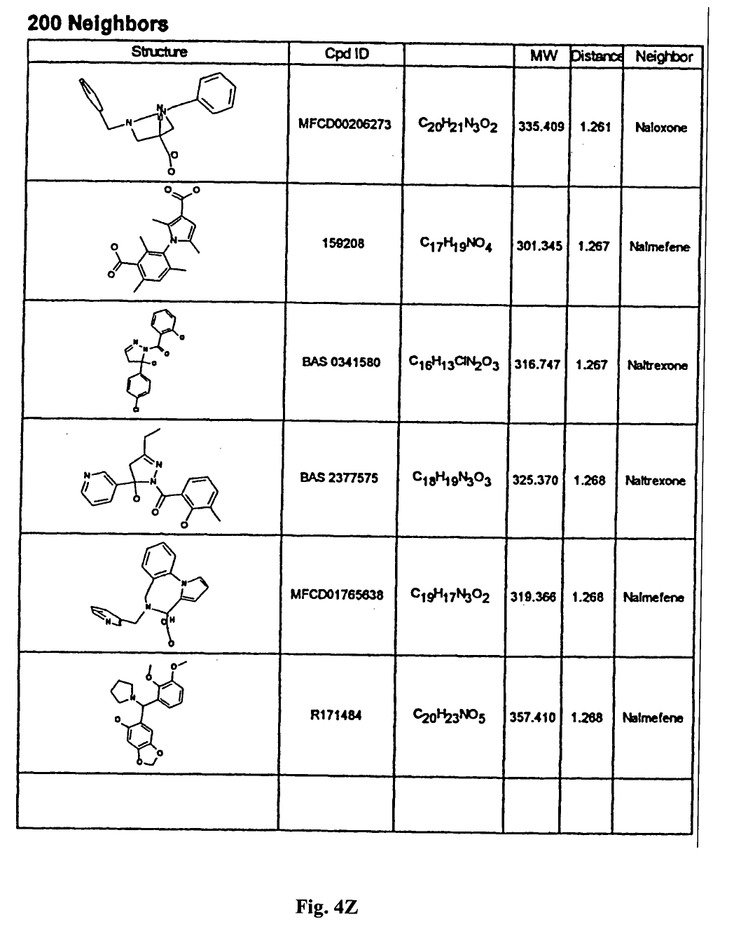

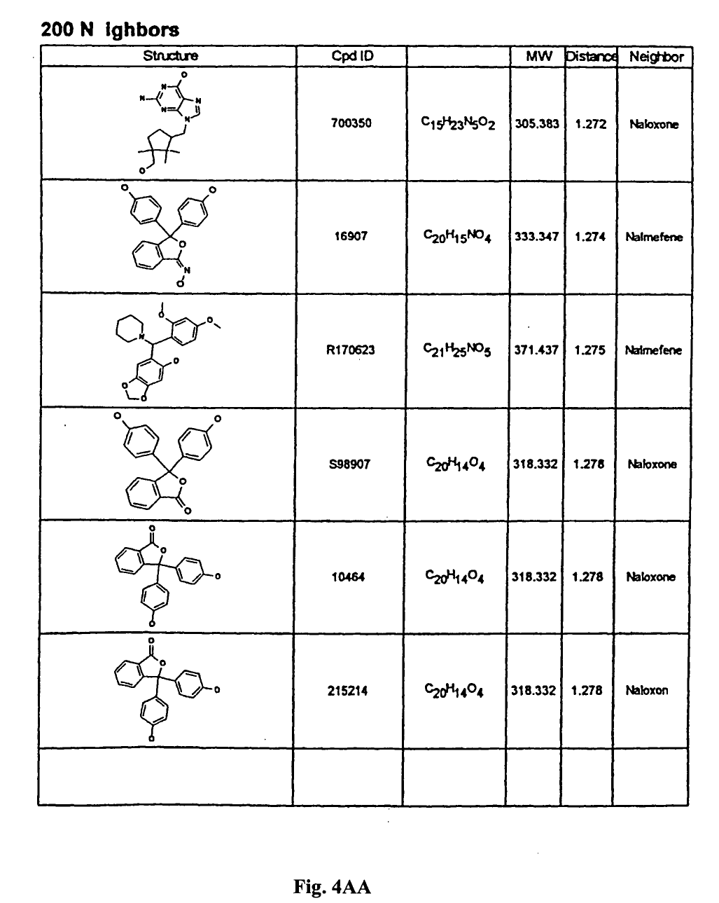

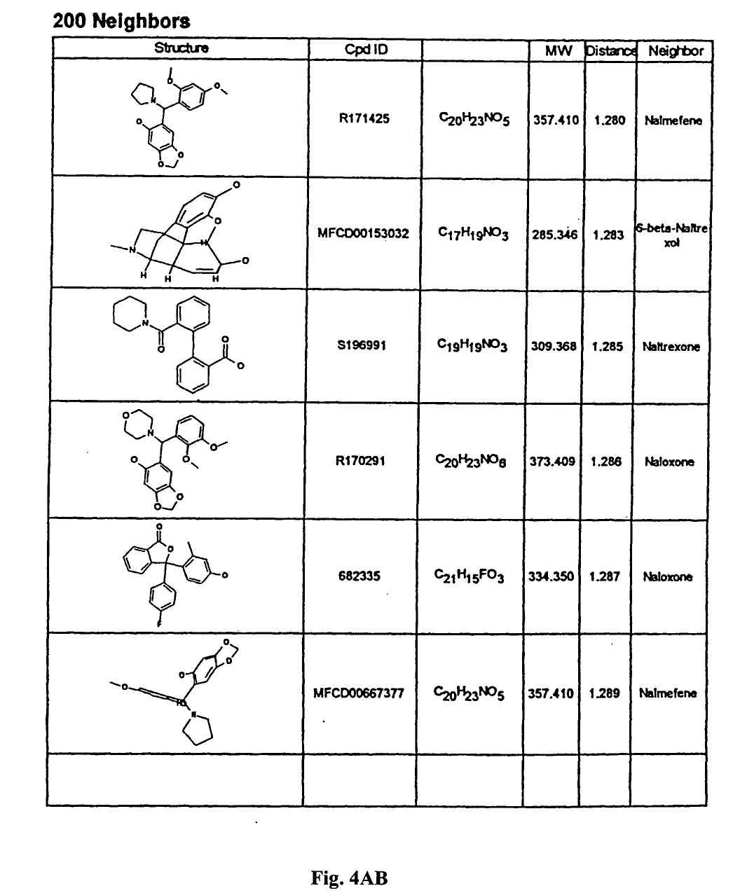

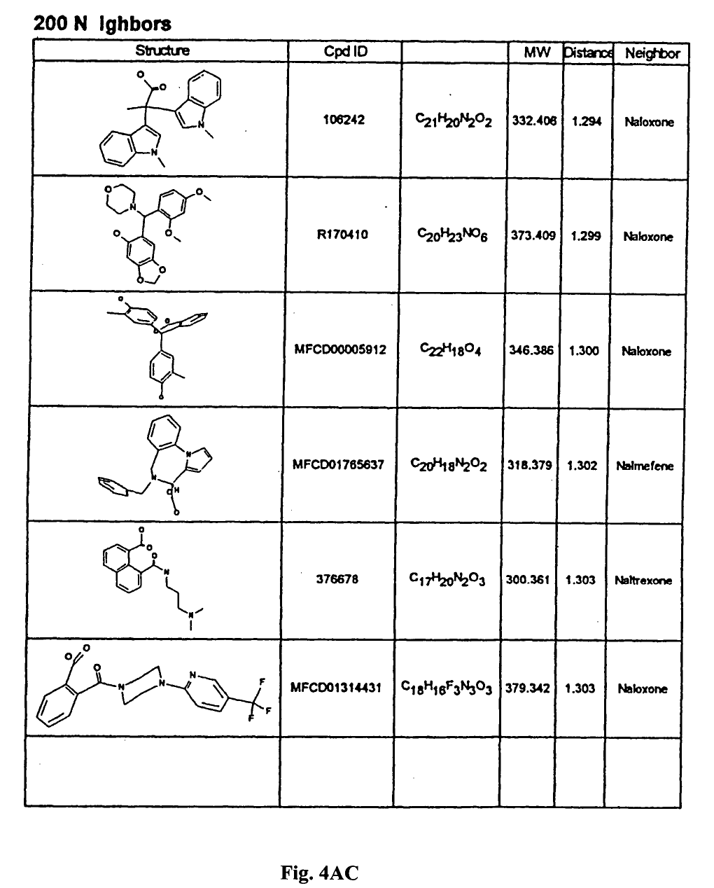

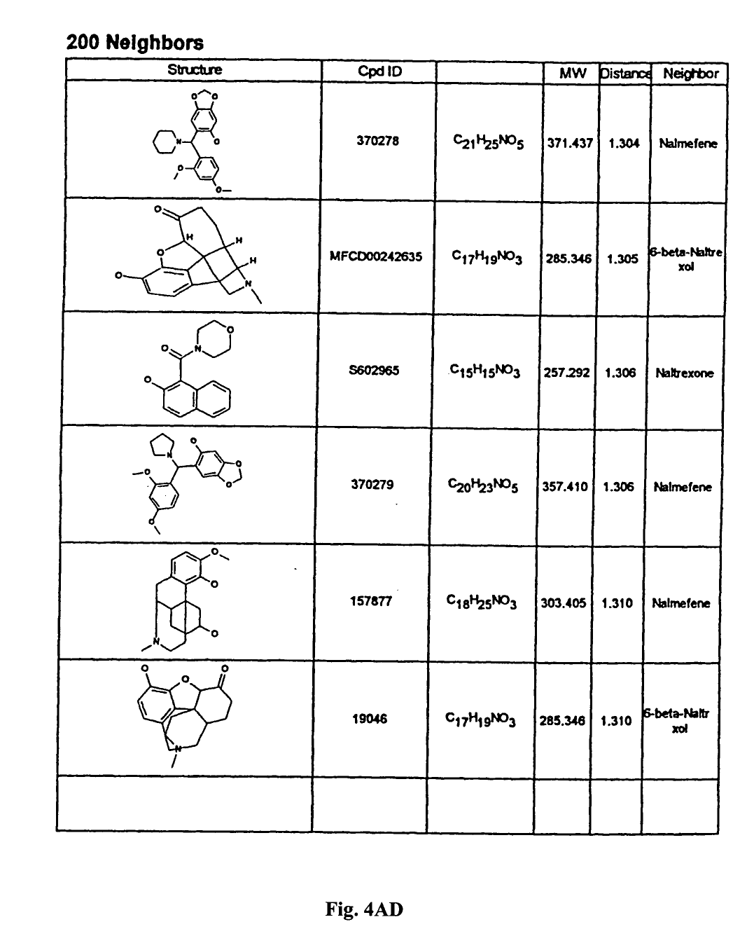

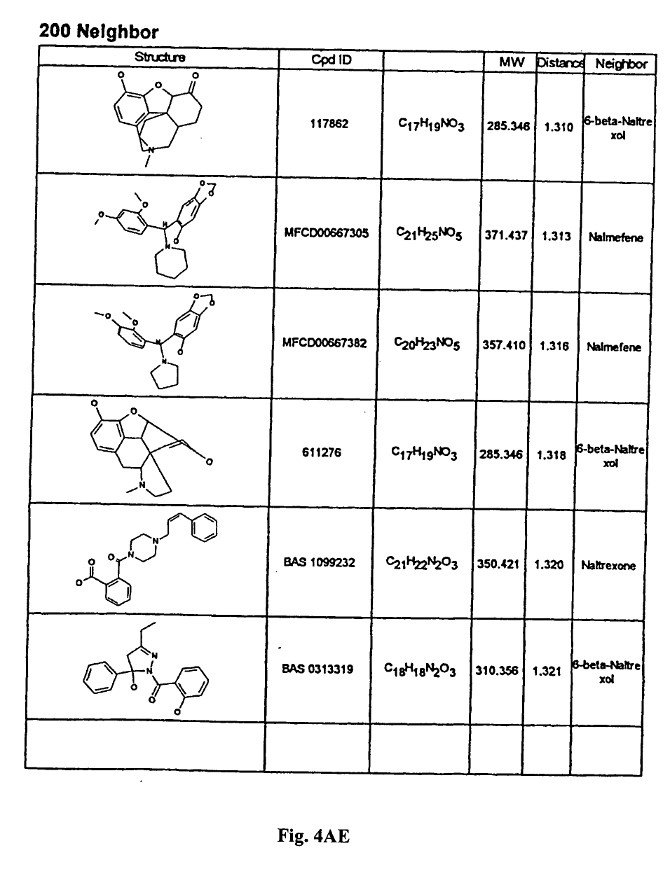

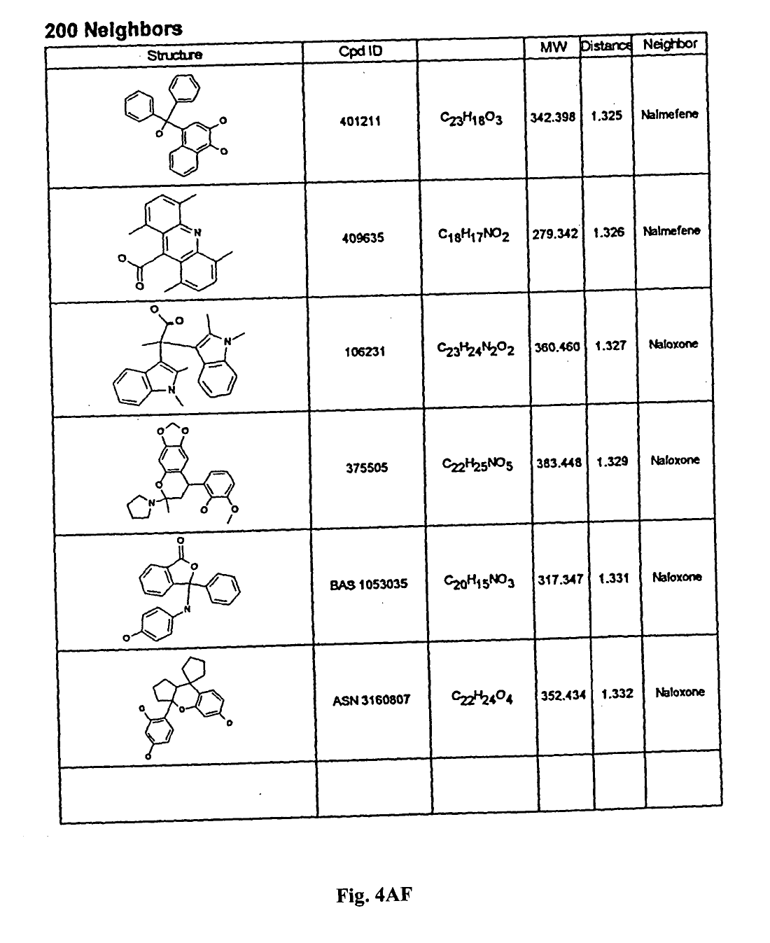

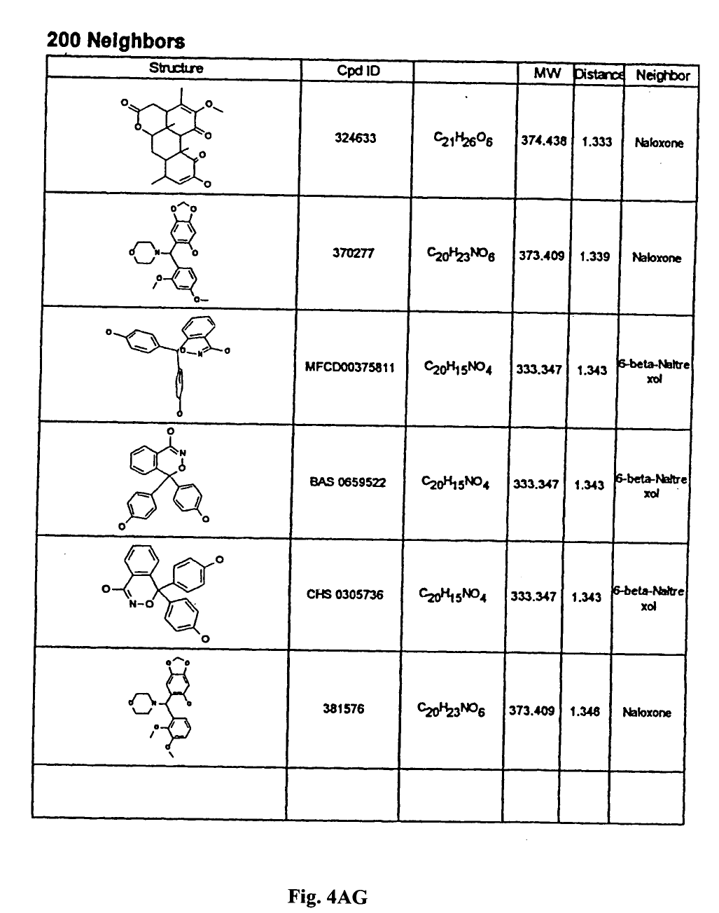

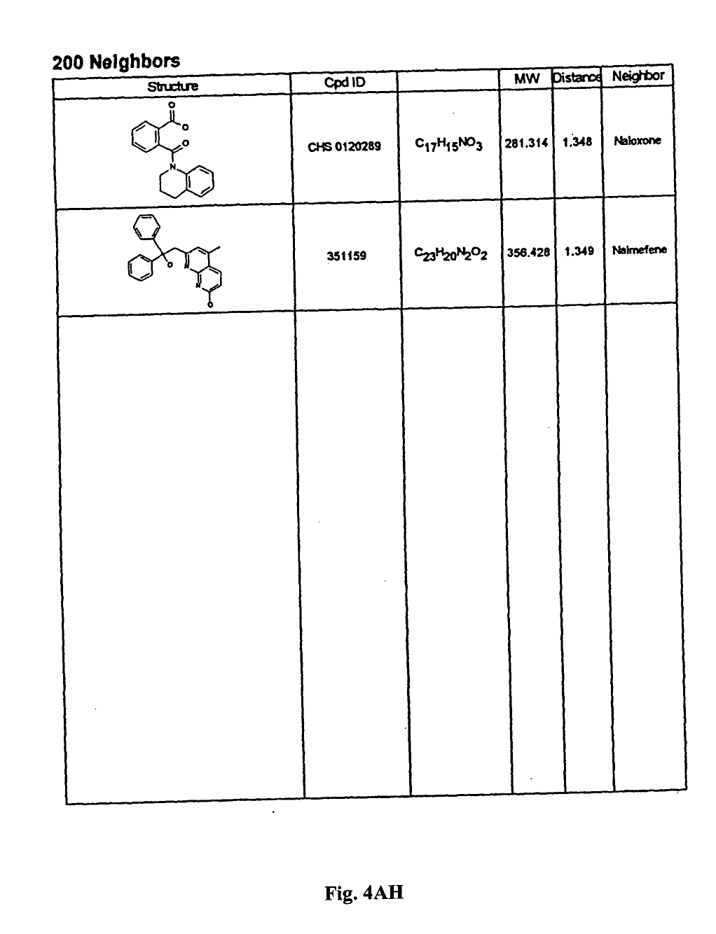

Molecular Orbital calculations were performed on the compounds using Spartan (Wavefunction, Inc.). There were no appreciable differences among the active compounds with respect to their electrostatic potentials. The electrostatic potential of nalmefene and naloxone are illustrated in FIGS. 3A and B respectively. The arrows indicate the hydroxyl group hydrogen-bond donor sites noted above.

Two views of an overlay of nalmefene and the low energy conformer of Kamm Compound 1 was prepared. The ring stacking structure predicted by Confort for the Kamm compounds embodies a conserved hydrophobic region shared by the both the Kamm compounds and the exemplary opioid compounds. The hydrogen-bond donor sites noted in the FIG. 3 are overlap the predicted hydrogen bonding sites of the Kamm compound. The nalmefene furan ring oxygen overlays on an aromatic ring in Kamm Compound 1, suggesting that the oxygen atom is not necessary for this activity.

In silico analyses of chemical compounds were conducted as follows: Diversity estimations were made on nalmefene, naloxone, naltrexone, 6-β-naltrexol, and the 16 Kamm et al structures using DiverseSolutions software from Tripos (R.S. Pearlman, UT-Austin). A chemistry space defined by approximately 900,000 chemical entities (several commercially available databases of compounds) was used as a reference. The commercial databases used as sources of the 900,000 chemical entities were MDL Information Systems (http://www.mdli.com), ACD Database (http://www.mdli.com/cgi/dynamic/product.html?uid=$uid&key=$key&id=17), NCI (http://dtp.nci.nih.gov/docs/3d_database/structural_information/smiles_strings.html), Aldrich (http://www.sigma-aldrich.com/saws.nsf/home?openframeset), ASINEx Ltd. (http://www.asinex.com), and Chemstar (http://www.chemstar.ru). A transporter-relevant subspace space was determined based on the former chemistry space, using the “B→A/A→B” efflux ratios to represent the activities. In order to have sufficient data, the Kamm et al data was combined with the high affinity/low capacity data provided for the exemplary opioid compounds. The 200 “nearest neighbors” are listed in Table 14 below. Note that in the Receptor-Relevant Subspace, the active compounds are focused in a small region of the overall chemistry space. Such compounds may be useful according to methods of the invention.

TABLE 14
200 Nearest Neighbors

| Rank | Database I.D. # | Distance to Exemplary compound | | |
|---|---|---|---|---|
| 1 | 70413 | 0.0096 | to | Naloxone |
| 2 | MFCD00133650 | 0.0184 | to | Nalmefene |
| 3 | 349115 | 0.4061 | to | Nalmefene |
| 4 | BAS 3387173 | 0.5101 | to | Naloxone |
| 5 | BAS 1002455 | 0.5195 | to | Naloxone |
| 6 | BAS 3387155 | 0.5243 | to | Naloxone |
| 7 | BAS 1268016 | 0.5345 | to | Naloxone |
| 8 | BAS 3387156 | 0.5412 | to | Naloxone |
| 9 | BAS 3387130 | 0.5462 | to | Naloxone |
| 10 | MFCD01935543 | 0.5507 | to | Naloxone |
| 11 | 688277 | 0.5913 | to | 6-β-Naltrexol |
| 12 | BAS 1002441 | 0.6179 | to | Naloxone |
| 13 | BAS 3386059 | 0.6369 | to | Naloxone |
| 14 | BAS 1003176 | 0.6370 | to | Naloxone |
| 15 | BAS 1004848 | 0.6434 | to | Naloxone |
| 16 | MFCD00273259 | 0.6436 | to | Nalmefene |
| 17 | MFCD00273270 | 0.6458 | to | Naloxone |
| 18 | MFCD00273266 | 0.6482 | to | Naloxone |
| 19 | BAS 3386023 | 0.6526 | to | Naloxone |
| 20 | BAS 2026128 | 0.6569 | to | Naloxone |
| 21 | 617005 | 0.6581 | to | 6-β-Naltrexol |
| 22 | MFCD00079194 | 0.6622 | to | 6-β-Naltrexol |
| 23 | 19045 | 0.6665 | to | 6-β-Naltrexol |
| 24 | 76021 | 0.6733 | to | Nalmefene |
| 25 | BAS 1002442 | 0.6770 | to | Naloxone |
| 26 | MFCD00271723 | 0.6822 | to | Naloxone |
| 27 | MFCD00273273 | 0.6884 | to | Nalmefene |
| 28 | MFCD00273264 | 0.6968 | to | Nalmefene |
| 29 | BAS 2026145 | 0.6977 | to | Naloxone |
| 30 | BAS 3387114 | 0.7036 | to | Naloxone |
| 31 | 376679 | 0.7051 | to | Naltrexone |
| 32 | 379963 | 0.7051 | to | Naltrexone |
| 33 | 157870 | 0.7144 | to | Nalmefene |
| 34 | MFCD00273274 | 0.7198 | to | Naloxone |
| 35 | MFCD00273260 | 0.7228 | to | Nalmefene |
| 36 | BAS 1003163 | 0.7272 | to | Naloxone |
| 37 | BAS 1003182 | 0.7388 | to | Naltrexone |
| 38 | BAS 0510629 | 0.7564 | to | Naltrexone |
| 39 | BAS 1002419 | 0.7571 | to | Naloxone |
| 40 | 18579 | 0.7600 | to | Nalmefene |
| 41 | 58796 | 0.7600 | to | Nalmefene |
| 42 | BAS 1004835 | 0.7634 | to | Naloxone |
| 43 | BAS 2004373 | 0.7646 | to | Naloxone |
| 44 | 693856 | 0.7680 | to | Nalmefene |
| 45 | MFCD01764789 | 0.7687 | to | Naloxone |
| 46 | MFCD00271738 | 0.7719 | to | Nalmefene |
| 47 | BAS 2025996 | 0.7741 | to | Naloxone |
| 48 | BAS 2282169 | 0.7798 | to | Nalmefene |
| 49 | MFCD00273268 | 0.7895 | to | Naloxone |
| 50 | MFCD00179880 | 0.7997 | to | Naloxone |
| 51 | BAS 1507170 | 0.8014 | to | Nalmefene |
| 52 | BAS 3386088 | 0.8017 | to | Naloxone |
| 53 | MFCD00272082 | 0.8183 | to | Nalmefene |
| 54 | MFCD00271113 | 0.8289 | to | 6-β-Naltrexol |
| 55 | 116054 | 0.8308 | to | 6-β-Naltrexol |
| 56 | BAS 1004837 | 0.8352 | to | Naloxone |
| 57 | 134536 | 0.8364 | to | 6-β-Naltrexol |
| 58 | 615801 | 0.8556 | to | Naltrexone |
| 59 | 404374 | 0.8695 | to | Nalmefene |
| 60 | MFCD00273318 | 0.8697 | to | Nalmefene |
| 61 | MFCD00271094 | 0.8774 | to | Nalmefene |
| 62 | 202587 | 0.8895 | to | Nalmefene |
| 63 | 693862 | 0.8919 | to | Nalmefene |
| 64 | MFCD00467140 | 0.9049 | to | Nalmefene |
| 65 | 693863 | 0.9093 | to | Naltrexone |
| 66 | MFCD00271196 | 0.9123 | to | Nalmefene |
| 67 | BAS 3386092 | 0.9195 | to | Naloxone |
| 68 | 693855 | 0.9235 | to | Nalmefene |
| 69 | BAS 3386091 | 0.9278 | to | Naloxone |
| 70 | MFCD00665833 | 0.9291 | to | Naltrexone |
| 71 | 404368 | 0.9412 | to | 6-β-Naltrexol |
| 72 | BAS 0606820 | 0.9478 | to | Naloxone |
| 73 | 693859 | 0.9485 | to | Nalmefene |
| 74 | BAS 0436353 | 0.9653 | to | Naloxone |
| 75 | MFCD00167445 | 0.9681 | to | Naltrexone |
| 76 | MFCD00667402 | 0.9742 | to | Nalmefene |
| 77 | MFCD002258126 | 0.9767 | to | Naloxone |
| 78 | MFCD00143186 | 0.9850 | to | Naltrexone |
| 79 | 119887 | 0.9932 | to | Naloxone |
| 80 | 404365 | 1.0016 | to | Nalmefene |
| 81 | MFCD01871411 | 1.0116 | to | Naloxone |
| 82 | 152720 | 1.0147 | to | 6-β-Naltrexol |
| 83 | 117581 | 1.0164 | to | Naloxone |
| 84 | 669466 | 1.0171 | to | Naloxone |
| 85 | MFCD00271129 | 1.0287 | to | Nalmefene |
| 86 | 689431 | 1.0350 | to | 6-β-Naltrexol |
| 87 | MFCD00056772 | 1.0390 | to | Nalmefene |
| 88 | MFCD00199295 | 1.0449 | to | Nalmefene |
| 89 | R191469 | 1.0457 | to | Nalmefene |
| 90 | 375504 | 1.0503 | to | Naloxone |
| 91 | 692397 | 1.0656 | to | Naloxone |
| 92 | MFCD00433684 | 1.0691 | to | Naloxone |
| 93 | 693860 | 1.0709 | to | Nalmefene |
| 94 | MFCD01764791 | 1.0725 | to | Naloxone |
| 95 | BAS 1519270 | 1.0776 | to | Naloxone |
| 96 | BAS 3385849 | 1.0828 | to | Naloxone |
| 97 | MFCD00673308 | 1.0866 | to | Nalmefene |
| 98 | 404356 | 1.0990 | to | Nalmefene |
| 99 | 43938 | 1.1067 | to | Nalmefene |
| 100 | 117181 | 1.1092 | to | Naltrexone |
| 101 | MFCD00094379 | 1.1109 | to | Nalmefene |
| 102 | 404369 | 1.1109 | to | 6-β-Naltrexol |
| 103 | 381577 | 1.1111 | to | Naloxone |
| 104 | S842214 | 1.1117 | to | Nalmefene |
| 105 | 134602 | 1.1123 | to | 6-β-Naltrexol |
| 108 | CHS 0316796 | 1.1130 | to | Naloxone |
| 107 | 134604 | 1.1147 | to | Nalmefene |
| 108 | R171697 | 1.1334 | to | Nalmefene |
| 109 | MFCD00667401 | 1.1343 | to | Nalmefene |
| 110 | S959863 | 1.1367 | to | 6-β-Naltrexol |
| 111 | 35545 | 1.1369 | to | 6-β-Naltrexol |
| 112 | 134598 | 1.1369 | to | 6-β-Naltrexol |
| 113 | S310778 | 1.1403 | to | Naloxone |
| 114 | 669800 | 1.1408 | to | Naloxone |
| 115 | BAS 0083962 | 1.1413 | to | Naltrexone |
| 116 | MFCD01765597 | 1.1424 | to | 6-β-Naltrexol |
| 117 | 682334 | 1.1427 | to | Naloxone |
| 118 | BAS 0631739 | 1.1428 | to | Nalmefene |
| 119 | MFCD00144882 | 1.1486 | to | 6-β-Naltrexol |
| 120 | MFCD00229975 | 1.1497 | to | Naloxone |
| 121 | R171700 | 1.1568 | to | Nalmefene |
| 122 | 134592 | 1.1633 | to | 6-β-Naltrexol |
| 123 | 401210 | 1.1662 | to | Nalmefene |
| 124 | BAS 2026074 | 1.1715 | to | Naltrexone |
| 125 | BAS 3050727 | 1.1767 | to | Nalmefene |
| 126 | BAS 0341630 | 1.1851 | to | Naloxone |
| 127 | 97817 | 1.1901 | to | Naloxone |
| 128 | ASN 3185453 | 1.1958 | to | Naloxone |
| 129 | 21257 | 1.1962 | to | 6-β-Naltrexol |
| 130 | 134601 | 1.2005 | to | 6-β-Naltrexol |
| 131 | BAS 2026075 | 1.2027 | to | 6-β-Naltrexol |
| 132 | BAS 1996620 | 1.2114 | to | 6-β-Naltrexol |
| 133 | MFCD01314356 | 1.2147 | to | Naloxone |
| 134 | BAS 2026097 | 1.2207 | to | Naltrexone |
| 135 | BAS 1914007 | 1.2210 | to | Naloxone |
| 136 | CHS 0003221 | 1.2266 | to | Naloxone |
| 137 | 667258 | 1.2274 | to | Naloxone |
| 138 | 37625 | 1.2351 | to | Nalmefene |
| 139 | BAS 1003093 | 1.2362 | to | 6-β-Naltrexol |
| 140 | 16468 | 1.2380 | to | Naloxone |
| 141 | CHS 0227049 | 1.2409 | to | Naloxone |
| 142 | BAS 0315050 | 1.2410 | to | Nalmefene |
| 143 | BAS 1289763 | 1.2421 | to | Naloxone |
| 144 | 349127 | 1.2429 | to | Naloxone |
| 145 | 635928 | 1.2496 | to | Nalmefene |
| 146 | BAS 2377555 | 1.2507 | to | 6-β-Naltrexol |
| 147 | MFCD00665835 | 1.2508 | to | Naltrexone |
| 148 | 47931 | 1.2547 | to | 6-β-Naltrexol |
| 149 | 76435 | 1.2572 | to | Nalmefene |
| 150 | 90558 | 1.2581 | to | Naloxone |
| 151 | MFCD00206273 | 1.2608 | to | Naloxone |
| 152 | 159208 | 1.2670 | to | Nalmefene |
| 153 | BAS 0341580 | 1.2672 | to | Naltrexone |
| 154 | BAS 2377575 | 1.2678 | to | Naltrexone |
| 155 | MFCD01765638 | 1.2681 | to | Nalmefene |
| 156 | R171484 | 1.2684 | to | Nalmefene |
| 157 | 700350 | 1.2716 | to | Naloxone |
| 158 | 16907 | 1.2740 | to | Nalmefene |
| 159 | R170623 | 1.2754 | to | Nalmefene |
| 160 | S98907 | 1.2776 | to | Naloxone |
| 161 | 10464 | 1.2777 | to | Naloxone |
| 162 | 215214 | 1.2777 | to | Naloxone |
| 163 | R171425 | 1.2802 | to | Nalmefene |
| 164 | MFCD00153032 | 1.2831 | to | 6-β-Naltrexol |
| 165 | S196991 | 1.2850 | to | Naltrexone |
| 166 | R170291 | 1.2863 | to | Naloxone |
| 167 | 682335 | 1.2867 | to | Naloxone |
| 168 | UFCD00667377 | 1.2889 | to | Nalmefene |
| 169 | 106242 | 12944 | to | Naloxone |
| 170 | R170410 | 1.2989 | to | Naloxone |
| 171 | MFCD0005912 | 1.2996 | to | Naloxone |
| 172 | MFCD01765637 | 1.3018 | to | Nalmefene |
| 173 | 376678 | 1.3028 | to | Naltrexone |
| 174 | MFCD01314431 | 1.3031 | to | Naloxone |
| 175 | 370278 | 1.3040 | to | Nalmefene |
| 176 | MFCD00242635 | 1.3054 | to | 6-β-Naltrexol |
| 177 | S602965 | 1.3058 | to | Naltrexone |
| 178 | 370279 | 1.3063 | to | Nalmefene |
| 179 | 157877 | 1.3099 | to | Nalmefene |
| 180 | 19046 | 1.3103 | to | 6-β-Naltrexol |
| 181 | 117862 | 1.3103 | to | 6-β-Naltrexol |
| 182 | MFCD00667305 | 1.3134 | to | Nalmefene |
| 183 | MFCD00667382 | 1.3161 | to | Nalmefene |
| 184 | 611276 | 1.3178 | to | 6-β-Naltrexol |
| 185 | BAS 1099232 | 1.3197 | to | Naltrexone |
| 186 | BAS 0313319 | 1.3206 | to | 6-β-Naltrexol |
| 187 | 401211 | 1.3254 | to | Nalmefene |
| 188 | 409635 | 1.3263 | to | Nalmefene |
| 189 | 106231 | 1.3271 | to | Naloxone |
| 190 | 375505 | 1.3289 | to | Naloxone |
| 191 | BAS 1053035 | 1.3309 | to | Naloxone |
| 192 | ASN 3160807 | 1.3316 | to | Naloxone |
| 193 | 324633 | 1.3331 | to | Naloxone |
| 194 | 370277 | 1.3392 | to | Naloxone |
| 195 | MFCD00375811 | 1.3428 | to | 6-β-Naltrexol |
| 196 | CHS 0305736 | 1.3435 | to | 6-β-Naltrexol |
| 197 | BAS 0659522 | 1.3435 | to | 6-β-Naltrexol |
| 198 | 381576 | 1.3461 | to | Naloxone |
| 199 | CHS 0120289 | 1.3484 | to | Naloxone |
| 200 | 351159 | 1.3490 | to | Nalmefene |

A pharmacophore for a drug transporter inhibitor useful according to the present invention contains the hydroxyl groups at the 14-position and 3-position as discussed above, the nitrogen, the hydrophobic region (tethered to the nitrogen), and the region of electron density at the 6-position. Other combinations of features are also possible as discussed below.

The distance between the hydroxyl groups in the pharmacophore (“H” of OH to “H” of OH) is approximately 7.4 Å. The equivalent distance in “Kamm 1” is ˜7.7 Å. These distances are to the Hydrogen atoms, rather than the H-bond acceptors in the binding site. The N-substituent lengths of nalmefene (from N to terminal Carbons) are ˜3.9 Å and ˜3.5 Å. N-substituent length of naloxone (from N to terminal Carbon) is ˜3.4 Å.

The three-dimensional coordinates of naltrexone are provided in Table 15.

TABLE 15
Three-Dimensional Coordinates

| ATOM | X | Y | Z | Type | Charge |
|---|---|---|---|---|---|
| C1 | −0.0352 | −0.1951 | 0.0725 | C.ar | 0.1489 |
| C2 | 2.0834 | −0.0915 | 0.6474 | C.3 | 0.1387 |
| C3 | 2.3288 | 1.3986 | 0.5409 | C.2 | 0.1298 |
| C4 | 2.7343 | 2.1393 | 1.7840 | C.3 | 0.0249 |
| C5 | 1.6213 | 1.9380 | 2.8395 | C.3 | −0.0154 |
| C6 | 1.5391 | 0.4338 | 3.2099 | C.3 | 0.0664 |
| C7 | 1.2934 | −0.4401 | 1.9514 | C.3 | 0.0294 |
| C8 | 0.3791 | 0.1181 | 4.2040 | C.3 | 0.0429 |
| C9 | −1.0383 | 0.5073 | 3.6641 | C.3 | 0.0052 |
| C10 | −1.2030 | 0.2284 | 2.1659 | C.ar | −0.0334 |
| C11 | −0.0782 | −0.1163 | 1.4337 | C.ar | −0.0151 |
| C12 | −2.4171 | 0.3074 | 1.4505 | C.ar | −0.0499 |
| C13 | −2.4130 | 0.2019 | 0.0328 | C.ar | −0.0203 |
| C14 | −1.2074 | 0.0000 | −0.6793 | C.ar | 0.1404 |
| O15 | 1.2170 | −0.4755 | −0.4637 | O.3 | −0.2867 |
| C16 | 1.3253 | −1.9545 | 2.2801 | C.3 | −0.0592 |
| N17 | 0.4895 | −1.3246 | 4.5611 | N.3 | −0.2960 |
| C18 | 0.3363 | −2.2765 | 3.4315 | C.3 | −0.0091 |
| O19 | 2.8028 | 0.1380 | 3.8337 | O.3 | −0.3969 |
| O20 | −1.1968 | 0.0000 | −2.0760 | O.3 | 0.3351 |
| O21 | 2.1919 | 2.0008 | −0.5126 | O.2 | −0.3894 |
| C22 | −0.1632 | −1.7771 | 5.8169 | C.3 | 0.0022 |
| C23 | 0.2667 | −0.9142 | 7.0296 | C.3 | −0.0282 |
| C24 | −0.5945 | −1.0908 | 8.2998 | C.3 | −0.0488 |
| C25 | −0.7018 | 0.2063 | 7.4700 | C.3 | −0.0488 |
| H26 | −3.3439 | 0.2757 | −0.5190 | H | 0.0719 |
| H27 | −3.3515 | 0.4481 | 1.9839 | H | 0.0519 |
| H28 | −0.7033 | −2.2458 | 3.0686 | H | 0.0417 |
| H29 | 0.5379 | −3.3100 | 3.7583 | H | 0.0417 |
| H30 | 1.0537 | −2.5464 | 1.3901 | H | 0.0165 |
| H31 | 2.3491 | −2.2448 | 2.5610 | H | 0.0165 |
| H32 | 3.7066 | 1.7640 | 2.1382 | H | 0.0495 |
| H33 | 2.8430 | 3.2119 | 1.5551 | H | 0.0495 |
| H34 | 0.6739 | 2.3152 | 2.4251 | H | 0.0308 |
| H35 | 1.8585 | 2.5217 | 3.7437 | H | 0.0308 |
| H36 | −1.2074 | 1.5867 | 3.7999 | H | 0.0488 |
| H37 | −1.8236 | −0.0234 | 4.2195 | H | 0.0488 |
| H38 | 3.0581 | −0.5987 | 0.5948 | H | 0.0780 |
| H39 | 0.5866 | 0.7227 | 5.1003 | H | 0.0510 |
| H40 | −0.3069 | 0.0000 | −2.4176 | H | 0.2424 |
| H41 | 2.8163 | −0.7158 | 4.2555 | H | 0.2089 |
| H42 | 0.1871 | −2.7925 | 6.0602 | H | 0.0429 |
| H43 | −1.2569 | −1.8218 | 5.7021 | H | 0.0429 |
| H44 | 1.3391 | −0.7446 | 7.2194 | H | 0.0313 |
| H45 | −1.6257 | 0.3467 | 6.8884 | H | 0.0268 |
| H46 | −0.2477 | 1.1098 | 7.9059 | H | 0.0268 |
| H47 | −1.4559 | −1.7752 | 8.2529 | H | 0.0268 |
| H48 | −0.0805 | −1.0045 | 9.2699 | H | 0.0268 |

Through the use of these coordinates a pharmacophore may be defined by: (1) a hydrogen bonding moiety at a three-dimensional location corresponding to the hydroxyl at position 3 of naltrexone; (2) a hydrogen bonding moiety at a three-dimensional location corresponding to the hydroxyl at position 14 of naltrexone; (3) a hydrophobic moiety at a three-dimensional location corresponding to the cyclopropyl moiety appended to the nitrogen of naltrexone; and (4) a region of electron density at a three-dimensional location corresponding to the ethylene moiety at 6-position of naltrexone.

All publications and patent applications mentioned in this specification are herein incorporated by reference to the same extent as if each individual publication of patent application was specifically and individually indicated to be incorporated by reference. U.S. application Ser. No. 10/000,113 (Filed Oct. 30, 2001-Docket No. 12971U.S.04); Ser. No. 10/003,215 (filed Oct. 30, 2001-Docket No. 13727U.S.01); ______ (filed May 30, 2002-Docket No. 13727U.S.02) are herein incorporated by reference.

The invention now being fully described, it will be apparent to one of ordinary skill in the art that many changes and modifications can be made thereto without departing from the spirit or scope of the appended claims.

What is claimed is:

1. A method of increasing the potency of an anti-microbial agent comprising co-administering to a subject infected with an ABC transporter-mediated multidrug resistant microbe:
(a) a dose of an anti-microbial agent, wherein the anti-microbial agent is a substrate of an ABC drug transporter; and
(b) a dose of an opioid inhibitor of the ABC drug transporter.

2. The method of claim 1, wherein the dose of the opioid inhibitor of the ABC drug transporter is sufficient to reduce efflux of the anti-microbial agent from the microbe, increase intracellular concentration of the anti-microbial agent in the microbe, or inhibit a drug transporter of the subject.

3. The method of claim 1, wherein the ABC drug transporter is a homologue of human PGP1a.

4. The method of claim 1, wherein the anti-microbial agent is one of the group of agents that are penicillins, cephalosporins, cycloserine, vancomycin, bacitracin, the azole antifungal agent, the polyene antifungal agents, the allylaminesthiocarbamates, chloramphenicol, the tetracyclines, erythromycin, clindamycin, the pristamycins, the aminoglysides, the rifamycins, the quinolones, trimethaprim, the sulfonamides, acyclovir, ganciclovir, zidovudine, lamivudine, daunomycin or doxorubicin.

5. The method of claim 1, wherein the opioid inhibitor of the ABC drug transporter is a compound of the formula: 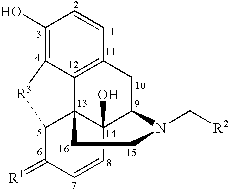
wherein R¹ is CH₂ or O;
wherein R² is a cycloalkyl, unsubstituted aromatic, alkyl or alkenyl; and
wherein R³ is O, CH₂ or NH.

6. The method of claim 1, wherein the opioid inhibitor of the ABC drug transporter is nalmefene, naltrexone or naloxone.

7. The method of claim 1, wherein the microbe causing the microbial infection is one of the groups of microbes that are Staphylococcus, Streptococcus, Micrococcus, Peptococcus, Peptostreptococcus, Enterococcus, Bacillus, Clostridium, LactobacilIus, Listeria, Erysipelothrix, Propionibacterium, Eubacterium, Corynebacterium, Pseudomonas, Plasmodium, Leishmania, Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, Saksenaea, Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, or Yersinia.

8. The method of claim 1, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

9. A method of increasing the potency of an anti-microbial agent comprising co-administering to a subject infected with an ABC transporter-mediated multidrug resistant microbe:
(a) a dose of an anti-microbial agent, wherein the anti-microbial agent is a substrate of an ABC drug transporter; and
(b) a dose of an opioid inhibitor of the ABC drug transporter.

10. The method of claim 9, wherein the dose of the opioid inhibitor of the ABC drug transporter is sufficient to increase the intracellular concentration of the anti-microbial agent in the microbe, reduce efflux of the anti-microbial agent from the microbe, or inhibit a drug transporter of the subject.

11. The method of claim 9, wherein the ABC drug transporter is a homologue of human PGP1a.

12. The method of claim 9, wherein the anti-microbial agent is one of the groups of agents that are penicillins, cephalosporins, cycloserine, vancomycin, bacitracin, the azole antifungal agent, the polyene antifungal agents, the allylaminesthiocarbamates, chloramphenicol, the tetracyclines, erythromycin, clindamycin, the pristamycins, the aminoglysides, the rifamycins, the quinolones, trimethaprim, the sulfonamides, acyclovir, ganciclovir, zidovudine, lamivudine, daunomycin or doxorubicin.

13. The method of claim 9, wherein the opioid inhibitor of the ABC drug transporter is a compound of the formula: 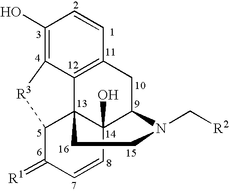
wherein R¹ is CH₂ or O;
wherein R² is a cycloalkyl, unsubstituted aromatic, alkyl or alkenyl; and
wherein R³ is O, CH₂ or NH.

14. The method of claim 9, wherein the opioid inhibitor of the ABC drug transporter is nalmefene, naltrexone or naloxone.

15. The method of claim 9, wherein the microbe causing the microbial infection is one of the groups of microbes that are Staphylococcus, Streptococcus, Micrococcus, Peptococcus, Peptostreptococcus, Enterococcus, Bacillus, Clostridium, LactobacilIus, Listeria, Erysipelothrix, Propionibacterium, Eubacterium, Corynebacterium, Pseudomonas, Plasmodium, Leishmania, Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, Saksenaea, Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, or Yersinia.

16. The method of claim 9, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

17. A composition for treating a microbial infection comprising:
(a) an anti-microbial agent, wherein the anti-microbial agent is a substrate of an ABC drug transporter; and
(b) an opioid inhibitor of the ABC drug transporter.

18. The composition of claim 17, wherein the ABC drug transporter is a homologue of human PGP1a.

19. The composition of claim 17, wherein the anti-microbial agent is one of the groups of agents that are penicillins, cephalosporins, cycloserine, vancomycin, bacitracin, the azole antifungal agent, the polyene antifungal agents, the allylaminesthiocarbamates, chloramphenicol, the tetracyclines, erythromycin, clindamycin, the pristamycins, the aminoglysides, the rifamycins, the quinolones, trimethaprim, the sulfonamides, acyclovir, ganciclovir, zidovudine, lamivudine, daunomycin or doxorubicin.

20. The composition of claim 17, wherein the opioid inhibitor of the ABC drug transporter is a compound of the formula: 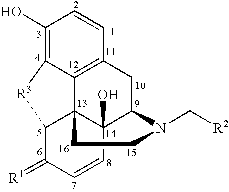
wherein R¹ is CH₂ or O;
wherein R² is a cycloalkyl, unsubstituted aromatic, alkyl or alkenyl; and
wherein R³ is O, CH₂ or NH.

21. The composition of claim 17, wherein the opioid inhibitor of the ABC drug transporter is nalmefene, naltrexone or naloxone.

22. The composition of claim 17, wherein the microbe causing the microbial infection is one of the groups of microbes that are Staphylococcus, Streptococcus, Micrococcus, Peptococcus, Peptostreptococcus, Enterococcus, Bacillus, Clostridium, LactobacilIus, Listeria, Erysipelothrix, Propionibacterium, Eubacterium, Corynebacterium, Pseudomonas, Plasmodium, Leishmania, Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, Saksenaea, Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, or Yersinia.

23. The composition of claim 17, wherein the opioid inhibitor of the drug transporter is nalmefene.

24. The composition of claim 17, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

25. A method of enhancing the anti-microbial activity of an anti-microbial agent against a microbe comprising:
contacting the microbe with the anti-microbial agent and an opioid inhibitor of an ABC drug transporter in an amount effective to inhibit a drug transporter in the microbe, wherein the microbe expresses an ABC drug transporter and the anti-microbial agent is a substrate of the ABC drug transporter.

26. The method of claim 25, wherein the ABC drug transporter is a homologue of human PGP1a.

27. The method of claim 25, wherein the anti-microbial agent is one of the groups of agents that are penicillins, cephalosporins, cycloserine, vancomycin, bacitracin, the azole antifungal agent, the polyene antifungal agents, the allylaminesthiocarbamates, chloramphenicol, the tetracyclines, erythromycin, clindamycin, the pristamycins, the aminoglysides, the rifamycins, the quinolones, trimethaprim, the sulfonamides, acyclovir, ganciclovir, zidovudine, lamivudine, daunomycin or doxorubicin.

28. The method of claim 25, wherein the opioid inhibitor of the ABC drug transporter is a compound of the formula: 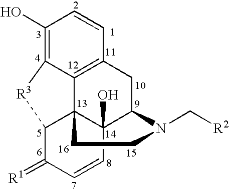
wherein R¹ is CH₂ or O;
wherein R² is a cycloalkyl, unsubstituted aromatic, alkyl or alkenyl; and
wherein R³ is O, CH₂ or NH.

29. The method of claim 25, wherein the opioid inhibitor of the ABC drug transporter is nalmefene, naltrexone or naloxone.

30. The method of claim 25, wherein the microbe causing the microbial infection is one of the groups of microbes that are Staphylococcus, Streptococcus, Micrococcus, Peptococcus, Peptostreptococcus, Enterococcus, Bacillus, Clostridium, LactobacilIus, Listeria, Erysipelothrix, Propionibacterium, Eubacterium, Corynebacterium, Pseudomonas, Plasmodium, Leishmania, Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, Saksenaea, Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, or Yersinia.

31. The method of claim 25, wherein the opioid inhibitor of the drug transporter is nalmefene.

32. The method of claim 25, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

33. A method of suppressing growth of a microbe expressing an ABC drug transporter protein comprising:
contacting the microbe with a sub-therapeutic amount of an anti-microbial agent in the presence of an opioid inhibitor of the ABC drug transporter.

34. The method of claim 33, wherein the ABC drug transporter is a homologue of human PGP1a.

35. The method of claim 33, wherein the anti-microbial agent is one of the groups of agents that are penicillins, cephalosporins, cycloserine, vancomycin, bacitracin, the azole antifungal agent, the polyene antifungal agents, the allylaminesthiocarbamates, chloramphenicol, the tetracyclines, erythromycin, clindamycin, the pristamycins, the aminoglysides, the rifamycins, the quinolones, trimethaprim, the sulfonamides, acyclovir, ganciclovir, zidovudine, lamivudine, daunomycin or doxorubicin.

36. The method of claim 33, wherein the opioid inhibitor of the ABC drug transporter is a compound of the formula: 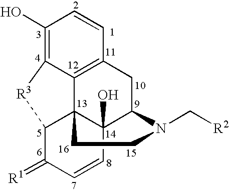
wherein R¹ is CH₂ or O;
wherein R² is a cycloalkyl, unsubstituted aromatic, alkyl or alkenyl; and
wherein R³ is O, CH₂ or NH.

37. The method of claim 33, wherein the opioid inhibitor of the ABC drug transporter is nalmefene, naltrexone or naloxone.

38. The method of claim 33, wherein the microbe is one of the groups of microbes that are Staphylococcus, Streptococcus, Micrococcus, Peptococcus, Peptostreptococcus, Enterococcus, Bacillus, Clostridium, LactobacilIus, Listeria, Erysipelothrix, Propionibacterium, Eubacterium, Corynebacterium, Pseudomonas, Plasmodium, Leishmania, Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, Saksenaea, Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, or Yersinia.

39. The method of claim 33, wherein the opioid inhibitor of the drug transporter is nalmefene.

40. The method of claim 33, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

41. A method of inhibiting a microbial P-glycoprotein homologue in a patient suffering from a microbial infection comprising administering to the patient a P-glycoprotein inhibiting amount of an inhibitor of an ABC drug transporter, wherein the inhibitor is nalmefene, naltrexone or naloxone, wherein the inhibitor is administered before, with, or after the administration to the patient of a therapeutic or sub-therapeutic amount of an anti-microbial agent.

42. The method of claim 41, wherein the ABC drug transporter is a homologue of human PGP1a.

43. The method of claim 41, wherein the anti-microbial agent is one of the groups of agents that are penicillins, cephalosporins, cycloserine, vancomycin, bacitracin, the azole antifungal agent, the polyene antifungal agents, the allylaminesthiocarbamates, chloramphenicol, the tetracyclines, erythromycin, clindamycin, the pristamycins, the aminoglysides, the rifamycins, the quinolones, trimethaprim, the sulfonamides, acyclovir, ganciclovir, zidovudine, lamivudine, daunomycin or doxorubicin.

44. The method of claim 41, wherein the microbe causing the microbial infection is one of the groups of microbes that are Staphylococcus, Streptococcus, Micrococcus, Peptococcus, Peptostreptococcus, Enterococcus, Bacillus, Clostridium, LactobacilIus, Listeria, Erysipelothrix, Propionibacterium, Eubacterium, Corynebacterium, Pseudomonas, Plasmodium, Leishmania, Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, Saksenaea, Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, or Yersinia.

45. A method of inhibiting a microbial P-glycoprotein homologue in a patient suffering from a microbial infection comprising administering to the patient a P-glycoprotein inhibiting amount of an inhibitor of an ABC drug transporter, wherein the inhibitor of the ABC drug transporter is a compound of the formula: 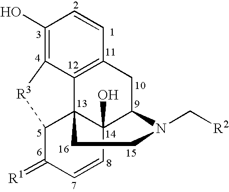
wherein R¹ is CH₂ or O;
wherein R² is a cycloalkyl, unsubstituted aromatic, alkyl or alkenyl; and
wherein R³is O, CH₂ or NH,
wherein the inhibitor of the ABC drug transporter is administered before, with, or after the administration to the patient of an anti-microbial effective amount of an anti-microbial agent.

46. The method of claim 45, wherein the anti-microbial agent is one of the groups of agents that are penicillins, cephalosporins, cycloserine, vancomycin, bacitracin, the azole antifungal agent, the polyene antifungal agents, the allylaminesthiocarbamates, chloramphenicol, the tetracyclines, erythromycin, clindamycin, the pristamycins, the aminoglysides, the rifamycins, the quinolones, trimethaprim, the sulfonamides, acyclovir, ganciclovir, zidovudine, lamivudine, daunomycin or doxorubicin.

47. The method of claim 45, wherein the microbe causing the microbial infection is one of the groups of microbes that are Staphylococcus, Streptococcus, Micrococcus, Peptococcus, Peptostreptococcus, Enterococcus, Bacillus, Clostridium, LactobacilIus, Listeria, Erysipelothrix, Propionibacterium, Eubacterium, Corynebacterium, Pseudomonas, Plasmodium, Leishmania, Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, Saksenaea, Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, or Yersinia.

48. A composition for the treatment of a microbial infection comprising:
(a) an opioid inhibitor of an ABC drug transporter; and
(b) an anti-microbial agent
wherein the opioid inhibitor of the ABC drug transporter is capable of inhibiting a drug transporter protein.

49. The composition of claim 48, wherein the ABC drug transporter is a homologue of human PGP1a.

50. The composition of claim 48, wherein the anti-microbial agent is one of the groups of microbes that are penicillins, cephalosporins, cycloserine, vancomycin, bacitracin, the azole antifungal agent, the polyene antifungal agents, the allylaminesthiocarbamates, chloramphenicol, the tetracyclines, erythromycin, clindamycin, the pristamycins, the aminoglysides, the rifamycins, the quinolones, trimethaprim, the sulfonamides, acyclovir, ganciclovir, zidovudine, lamivudine, daunomycin and doxorubicin.

51. The composition of claim 48, wherein the opioid inhibitor of the ABC drug transporter is a compound of the formula: 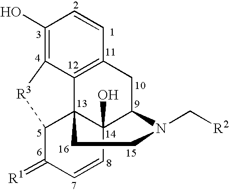
wherein R¹ is CH₂ or O;
wherein R² is a cycloalkyl, unsubstituted aromatic, alkyl or alkenyl; and
wherein R³ is O, CH₂ or NH.

52. The composition of claim 48, wherein the opioid inhibitor of the ABC drug transporter is nalmefene, naltrexone or naloxone.

53. The composition of claim 48, wherein the microbe causing the microbial infection is one of the groups of microbes that are Staphylococcus, Streptococcus, Micrococcus, Peptococcus, Peptostreptococcus, Enterococcus, Bacillus, Clostridium, LactobacilIus, Listeria, Erysipelothrix, Propionibacterium, Eubacterium, Corynebacterium, Pseudomonas, Plasmodium, Leishmania, Absidia, Aspergillus, Basidiobolus, Blastomyces, Candida, Coccidioides, Conidiobolus, Cryptococcus, Cunninghamella, Histoplasma, Mortierella, Mucor, Paracoccioides, Rhizopus, Saksenaea, Acidarninococcus, Acinetobacter, Aeromonas, Alcaligenes, Bacteroides, Bordetella, Branhamella, Brucella, Calymmatobacterium, Carnpylobacter, Cardiobacterium, Chromobacterium, Citrobacter, Edwardsiella, Enterobacter, Escherichia, Flavobacterium, Francisella, Fusobacterium, Haermophilus, Klebsiella, Legionella, Moraxella, Morganella, Neisseria, Pasturella, Plesiornonas, Proteus, Providencia, Pseudomonas, Salmonella, Serratia, Shigella, Streptobacillus, Veillonella, Vibrio, or Yersinia.

54. The composition of claim 48, wherein the opioid inhibitor of the drug transporter is nalmefene.

55. The composition of claim 48, wherein the opioid inhibitor of the drug transporter is a compound having the pharmacophore defined by:
a hydrogen bonding moiety at a three-dimensional location corresponding to the hydroxyl at position 3 of naltrexone;
a hydrogen bonding moiety at a three-dimensional location corresponding to the hydroxyl at position 14 of naltrexone;
a hydrophobic moiety at a three-dimensional location corresponding to the cyclopropyl moiety appended to the nitrogen of naltrexone; and
a region of electron density at a three-dimensional location corresponding to the ethylene moiety at 6-position of naltrexone.

56. A method of identifying a compound for improved treatment of microbial infections comprising:
(a) identifying an anti-microbial agent;
(b) assaying the ability of the therapeutic agent to be transported across a membrane by an ABC protein; and
(c) repeating the transport assay to determine whether addition of an opioid inhibitor of an ABC drug transporter inhibits transport of the therapeutic agent across the membrane,
whereby the compound that is transported by an ABC protein and whose ABC protein-mediated transport is inhibited by the opioid inhibitor of the ABC drug transporter is identified.

57. A method of enhancing the potency of an anti-microbial agent identified by the method of claim 56 comprising:
co-administering a therapeutic amount of the anti-microbial agent and an amount of an opioid inhibitor of an ABC drug transporter capable of inhibiting a drug transporter, wherein the amount of the opioid inhibitor of the ABC drug transporter is sufficient to reduce transport of the anti-microbial agent across a biological membrane.

58. A method for screening for an opioid inhibitor of an ABC drug transporter, comprising determining whether a potential opioid inhibitor inhibits growth of a microbial cell in the presence of sub-therapeutic amount of anti-microbial agent,
wherein the microbial cell expresses an ABC drug transporter, and
wherein said determining comprises comparing the growth of the microbial cell which expresses the ABC drug transporter, with growth of a second microbial cell which does not produce the ABC drug transporter, wherein the first and second microbial cells are grown in the presence of the sub-therapeutic amount of the anti-microbial agent.

59. A method for screening for an opioid inhibitor of an ABC drug transporter, comprising:
contacting a potential opioid inhibitor of an ABC drug transporter protein with the ABC drug transporter protein in the presence of a compound that is nalmefene, naltrexone or naloxone, wherein the compound is detectably labeled;
measuring the amount of detectably labeled compound bound to the ABC drug transporter; and
comparing the measured amount to the amount of detectably labeled compound bound by the ABC drug transporter when the drug transporter is contacted with the compound alone,
whereby a measured amount lower than the amount of compound bound to the ABC drug transporter when contacted alone identifies an opioid inhibitor of the ABC drug transporter.

60. The method of claim 59, wherein the potential opioid inhibitor of the ABC drug transporter is nalmefene.

61. A method of treating a microbial infection in a subject, comprising administering to the subject suffering from the infection an anti-microbial agent and an ABC drug transporter inhibitor in an amount sufficient to increase the intracellular concentration of the anti-microbial agent in the microbe,
wherein the ABC drug transporter inhibitor increases the susceptibility of the microbe to the anti-microbial agent, and
wherein the ABC drug transporter inhibitor is nalmefene, naltrexone or naloxone.

62. A method of treating a microbial infection in a subject, comprising administering to the subject suffering from the infection an anti-microbial agent and an ABC drug transporter inhibitor in an amount sufficient to increase the intracellular concentration of the anti-microbial agent in the microbe,
wherein the ABC drug transporter inhibitor increases the susceptibility of the microbe to the anti-microbial agent, and
wherein the ABC drug transporter inhibitor is a compound of the formula: 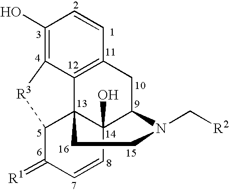
wherein R¹ is CH₂ or O;
wherein R² is a cycloalkyl, unsubstituted aromatic, alkyl or alkenyl; and
wherein R³ is O, CH₂ or NH.

63. A method of decreasing the toxicity of an anti-microbial agent comprising co-administering:
(a) a therapeutic or sub-therapeutic dose of the anti-microbial agent, wherein the anti-microbial agent is a substrate of an ABC drug transporter, and
(b) a dose of an opioid inhibitor of the ABC drug transporter.

64. A method of enhancing the potency of an anti-microbial agent comprising co-adminsitering:
(a) a therapeutic or sub-therapeutic dose of the anti-microbial agent, wherein the anti-microbial agent is a substrate of an ABC drug transporter, and
(b) a dose of an opioid inhibitor of the ABC drug transporter.

65. A method of increasing the potency of an anti-microbial agent comprising co-administering to patient infected with an ABC transporter-mediated multidrug resistant microbe:
(a) a dose of an anti-microbial agent, wherein the anti-microbial agent is a substrate of an ABC drug transporter; and
(b) a dose of an opioid inhibitor of the ABC drug transporter, wherein the dose of the opioid inhibitor of the ABC drug transporter is sufficient to reduce efflux of the anti-microbial agent from the microbe.

66. A method of increasing the potency of an anti-microbial agent comprising co-administering to patient infected with an ABC transporter-mediated multidrug resistant microbe:
(a) a dose of an anti-microbial agent, wherein the anti-microbial agent is a substrate of an ABC drug transporter; and
(b) a dose of an opioid inhibitor of the ABC drug transporter, wherein the dose of the opioid inhibitor of the ABC drug transporter is sufficient to increase the intracellular concentration of the anti-microbial agent in the microbe.
